SEQUENCE LISTING (83 sequences)

SEQ ID NO: 1 - protein, 303 aa, Ophiostoma novo-ulmi
MAYMSRRESINPWILTGFADAEGSFLLRIRNNNKSSVGYSTELGFQITLHNKDKSILENIQSTWKVGVIANSGDNAVSLKVTRFEDLKVIIDHFEKYPLITQKLGDYMLFKQAFCVMENKEHLKINGIKELVRIKAKLNWGLTDELKKAFPEIISKERSLINKNIPNFKWLAGFTSGEGCFFVNLIKSKSKLGVQVQLVFSITQHIKDKNLMNSLITYLGCGYIKEKNKSEFSWLDFVVTKFSDINDKIIPVFQENTLIGVKLEDFEDWCKVAKLIEEKKHLTESGLDEIKKIKLNMNKGRVF

SEQ ID NO: 2 - protein, 303 aa, Ophiostoma novo-ulmi
MAYMSRRESINPWILTGFADAEGSFLLRIRNNNKSSVGYSTELGFQITLHNKDKSILENIQSTWKVGVIANSGDNAVSLKVTRFEDLKVIIDHFEKYPLITQKLGDYKLFKQAFSVMENKEHLKENGIKELVRIKAKLNWGLTDELKKAFPENISKERSLINKNIPNFKWLAGFTSGEGCFFVNLIKSKSKLGVQVQLVFSITQHIKDKNLMNSLITYLGCGYIKEKNKSEFSWLDFVVTKFSDINDKIIPVFQENTLIGVKLEDFEDWCKVAKLIEEKKHLTESGLDEIKKIKLNMNKGRVF

SEQ ID NO: 3 - protein, 303 aa, Ophiostoma novo-ulmi
  MOD_RES (1)..(3): Any amino acid or absent
XXXMSRRESINPWILTGFADAEGSFLLRIRNNNKSSVGYSTELGFQITLHNKDKSILENIQSTWKVGVIANSGDNAVSLKVTRFEDLKVIIDHFEKYPLITQKLGDYKLFKQAFSVMENKEHLKENGIKELVRIKAKLNWGLTDELKKAFPENISKERSLINKNIPNFKWLAGFTSGEGCFFVNLIKSKSKLGVQVQLVFSITQHIKDKNLMNSLITYLGCGYIKEKNKSEFSWLDFVVTKFSDINDKIIPVFQENTLIGVKLEDFEDWCKVAKLIEEKKHLTESGLDEIKKIKLNMNKGRVF

SEQ ID NO: 4 - protein, 303 aa, Ophiostoma novo-ulmi
  MOD_RES (1)..(4): Any amino acid or absent
  MOD_RES (302)..(303): Any amino acid or absent
XXXXSRRESINPWILTGFADAEGSFLLRIRNNNKSSVGYSTELGFQITLHNKDKSILENIQSTWKVGVIANSGDNAVSLKVTRFEDLKVIIDHFEKYPLITQKLGDYKLFKQAFSVMENKEHLKENGIKELVRIKAKLNWGLTDELKKAFPENISKERSLINKNIPNFKWLAGFTSGEGCFFVNLIKSKSKLGVQVQLVFSITQHIKDKNLMNSLITYLGCGYIKEKNKSEFSWLDFVVTKFSDINDKIIPVFQENTLIGVKLEDFEDWCKVAKLIEEKKHLTESGLDEIKKIKLNMNKGRXX

SEQ ID NO: 5 - protein, 303 aa, Ophiostoma novo-ulmi
  MOD_RES (1)..(8): Any amino acid or absent
  MOD_RES (302)..(303): Any amino acid or absent
XXXXXXXXSINPWILTGFADAEGSFLLRIRNNNKSSVGYSTELGFQITLHNKDKSILENIQSTWKVGVIANSGDNAVSLKVTRFEDLKVIIDHFEKYPLITQKLGDYKLFKQAFSVMENKEHLKENGIKELVRIKAKLNWGLTDELKKAFPENISKERSLINKNIPNFKWLAGFTSGEGCFFVNLIKSKSKLGVQVQLVFSITQHIKDKNLMNSLITYLGCGYIKEKNKSEFSWLDFVVTKFSDINDKIIPVFQENTLIGVKLEDFEDWCKVAKLIEEKKHLTESGLDEIKKIKLNMNKGRXX

SEQ ID NO: 6 - protein, 303 aa, Artificial Sequence
  Synthesized I-OnuI variant BTK L4 V25
  MOD_RES (1)..(8): Any amino acid or absent
  MOD_RES (302)..(303): Any amino acid or absent
XXXXXXXXSINPWILTGFADAEGWFLLVIRNSNTVKVGYRTLLSFGIELHNKDKSILENIQSTWKVGKISNSGDHYVRLTVSRFEDLKVIIDHFEKYPLITQKLGDYKLFKQAFSLMENKEHLKENGIKELVRIKAKMNWGLNDELKKAFPENISKERPLINKNIPNLKWLAGFTSGDGSFYVNLVKGKNTTRVTVQLVFQITQHIKDKNLMNSLITYLGCGYILEKNVSERSFLQFRVTKFSDINDKIIPVFQENTLIGVKLEDFEDWCKVAKLIEEKKHLTESGLDEIKKIKLNMNKGRXX

SEQ ID NO: 7 - protein, 303 aa, Artificial Sequence
  Synthesized I-OnuI variant BTK EL4 V34
  MOD_RES (1)..(8): Any amino acid or absent
  MOD_RES (302)..(303): Any amino acid or absent
XXXXXXXXSINPWILTGFADAEGWFLLVIRNSNTVKVGYRTLLSFGIELHNKDKSILENIQSTWKVGKISNSGDHYVRLTVSRFEDLKVIIDHFEKYPLITQKLGDYKLFKQAFSLMENKEHLKENGIKELVRIRAKMNWGLNDELKKAFPENISKERPLINKNIPNLKWLAGFTSGDGSFYVNLVKGKNTTRVTVQLVFQITQHIKDKNLMNSLITYLGCGYILEKNVSERSFLQFRVTKFSDIKDKIIPVFQENTLIGVKLEDFEDWCKVAKLIEEKKHLTESGLDEIKKIKLNMNKGRXX

SEQ ID NO: 8 - protein, 303 aa, Artificial Sequence
  Synthesized I-OnuI variant BTK EL V42
  MOD_RES (1)..(8): Any amino acid or absent
  MOD_RES (302)..(303): Any amino acid or absent
XXXXXXXXSINPWILTGFADAEGWFLLDIRNSNTVKVGYRTLLSFGIELHNKDKSILENIQSTWKVGKIRNSGDRYVSLTVSRFEDLKVIIDHFEKYPLITQKLGDYKLFKQAFSLMENKEHLKENGIKELVRIKAKMNWGLNDELKKAFPENISKERPLINKSIPNLKWLAGFTSGDGSFYVNLVKGKNTTRVTVQLVFQITQHIKDKYLMNSLITYLGCGYILEKNVSERSFLQFRVTKFSDINDKIIPVFQENTLIGVKLEDFEDWCKVAKLIEEKKHLTESGLDEIKKIKLNMNKGRXX

SEQ ID NO: 9 - protein, 303 aa, Artificial Sequence
  Synthesized I-OnuI variant BTK EL4 V5
  MOD_RES (1)..(8): Any amino acid or absent
  MOD_RES (302)..(303): Any amino acid or absent
XXXXXXXXSINPWILTGFADAEGWFLLVIRNSNTVKVGYRTLLSFGIELHNKDKSILENIQSTWKVGKISNSGDHYVRLTVSRFEDLKVIIDHFEKYPLITQKLGDYKLFKQAFSVMENKEHLKENGIKELVRIKAKMNWGLNDELKKAFPENISKERPLINKNIPNLKWLAGFTSGDGTFYVNLIKGKNTTRVYVQLVFGITQHIKDKNLMNSLITYLGCGYILEKNVSERSFLQFRVTKFSDINDKIIPVFQENTLIGVKLEDFEDWCKVAKLIEEKKHLTESGLDEIKKIKLNMNKGRXX

SEQ ID NO: 10 - protein, 303 aa, Artificial Sequence
  Synthesized I-OnuI variant BTK EL4 V4
  MOD_RES (1)..(8): Any amino acid or absent
  MOD_RES (302)..(303): Any amino acid or absent
XXXXXXXXSINPWILTGFADAEGWFLLVIRNSNTVKVGYRTLLSFGIELHNKDKSILENIQSTWKVGKISNSGDHYVRLTVSRFEDLKVIIDHFEKYPLITQKLGDYKLFKQAFSLMENKEHLKENGIKELVRIKAKMNWGLNDELKKAFPENISKERPLINKNIPNLKWLAGFTSGDGSFYVNLIKGKNTTRVTVQLVFQITQHIKDKNLMNSLITYLGCGYILEKNVSERSFLQFRVTKFSDINDKIIPVFQENTLIGVKLEDFEDWCKVAKLIEEKKHLTESGLDEIKKIKLNMNKGRXX

SEQ ID NO: 11 - protein, 303 aa, Artificial Sequence
  Synthesized I-OnuI variant BTK EL5N V3
  MOD_RES (1)..(8): Any amino acid or absent
  MOD_RES (302)..(303): Any amino acid or absent
XXXXXXXXSINPWILTGFADAEGWFLLDIRNSNTVKVGYRTLLSFGIELHNKDKSILENIQSTWKVGKISNSGDHYVRLTVSRFEDLKVIIDHFEKYPLITQKLGDYKLFKQAFSLMENKEHLKENGIKELVRIKAKMNWGLNDELKKAFPENISKERPLINKNIPNLKWLAGFTSGDGTFYVNLIKGKNTTRVYVQLVFGITQHIKDKNLMNSLITYLGCGYILEKNVSERSFLQFRVTKFSDINDKIIPVFQENTLIGVKLEDFEDWCKVAKLIEEKKHLTESGLDEIKKIKLNMNKGRXX

SEQ ID NO: 12 - protein, 303 aa, Artificial Sequence
  Synthesized I-OnuI variant BTK EL5N V7
  MOD_RES (1)..(8): Any amino acid or absent
  MOD_RES (302)..(303): Any amino acid or absent
XXXXXXXXSINPWILTGFADAEGWFMLDIRNSNTVKVGYRTLLSFGIELHNKDKSILENIQSTWKVGKIRNSGDRYVSLTVSRFEDLKVIIDHFEKYPLITQKLGDYKLFKQAFSLMENKEHLKENGIKELVRIKAKMNWGLNDELEKAFPENISKERPLINKNIPNLKWLAGFTSGDGTFYVNLIKGKNTTRVYVQLVFGITQHIKDKNLMNSLITYLGCGYILEKNVSERSFLQFRVTKFSDINDKIIPVFQENTLIGVKLEDFEDWCKVAKLIEEKKHLTESGLDEIKKIKLNMNKGRXX

SEQ ID NO: 13 - protein, 303 aa, Artificial Sequence
  Synthesized I-OnuI variant BTK EL5 V12
  MOD_RES (1)..(8): Any amino acid or absent
  MOD_RES (302)..(303): Any amino acid or absent
XXXXXXXXSINPWILTGFADAEGWFSLVIRNSNTVKVGYRTLLSFGIELHNKDKSILENIQSTWKVGKISNSGDRAVRLTVTRFGDLKVIIDHFEKYPLITQKLGDYKLFKQAFSLMENKEHLKENGIKELVRIKAKMNWGLNDELKKAFPENISKERPLINKNIPNLKWLAGFTSGDGTFYVNLIKGKNTTRVYVQLVFGITQHIKDKNLMNSLITYLGCGYILEKNVSERSFLQFRVTKFSDINDKIIPVFQENTLIGVKLEDFEDWCKVAKLIEEKKHLTESGLDEIKKIKLNMNKGRXX

SEQ ID NO: 14 - protein, 303 aa, Artificial Sequence; identical to SEQ ID NO: 13
  Synthesized I-OnuI variant BTK EL5 V17
  MOD_RES (1)..(8): Any amino acid or absent
  MOD_RES (302)..(303): Any amino acid or absent

SEQ ID NO: 15 - protein, 303 aa, Artificial Sequence
  Synthesized I-OnuI variant BTK EL5 V18
  MOD_RES (1)..(8): Any amino acid or absent
  MOD_RES (302)..(303): Any amino acid or absent
XXXXXXXXSINPWILTGFADAEGWFLLVIRNSNTVKVGYRTLLSFGIELHNKDKSILENIQSTWKVGKISNSGDHYVRLTVSRFEDLKVIIDHFEKYPLITQKLGDYKLFKQAFSLMENKEHLKENGIKELVRIKAKMNWGLNDELKKAFPENISKERPLINKNIPNLKWLAGFTSGDGTFYVNLIKGKNTTRVYVQLVFGITQHIKDKNLMNSLITYLGCGYILEKNVSERSFLQFRVTKFSDINDKIIPVFQENTLIGVKLEDFEDWCKVAKLIEEKKHLTESGLDEIKKIKLNMNKGRXX

SEQ ID NO: 16 - protein, 303 aa, Artificial Sequence
  Synthesized I-OnuI variant BTK EL5 V21
  MOD_RES (1)..(8): Any amino acid or absent
  MOD_RES (302)..(303): Any amino acid or absent
XXXXXXXXSINPWILTGFADAEGWFSLVIRNSNTVKVGYRTLLSFGIELHNKDKSILENIRSTWKVGKISNSGDRAVRLTVTRFEDLKVIIDHFEKYPLITQKLGDYKLFKQAFSLMENKEHLKENGIKELVRIKAKMNWGLNDELKKAFPENISKERPLINKNIPNLKWLAGFTSGDGSFYVNLIKGKNTTRVYVQLVFGITQHIKDKNLMNSLITYLGCGYILEKNVSERSFLQFRVTKFSDINDKIIPVFQENTLIGVKLEDFEDWCKVAKLIEEKKHLTESGLDEIKKIKLNMNKGRXX

SEQ ID NO: 17 - protein, 303 aa, Artificial Sequence
  Synthesized I-OnuI variant BTK EL5N V44
  MOD_RES (1)..(8): Any amino acid or absent
  MOD_RES (302)..(303): Any amino acid or absent
XXXXXXXXSINPWILTGFADAEGWFLLDIRNSNTVKVGYRTLLSFGIELHNKDKSILENIQSTWKVGKISNSGDHYVRLTVSRFEDLKVIIDHFEKYPLITQKLGDYKLFKQAFSLMENKEHLKENGIKELVRIKAKMNWGLNDELKKAFPENISKERPLVNKNIPNLKWLAGFTSGDGTFYVNLIKGKNTTRVYVQLVFGITQHIKDKNLMNSLITYLGCGYILEKNVSERSFLQFRVTKFSDINDKIIPVFQENTLIGVKLEDFEDWCKVAKLIEEKKHLTESGLDEIKKIKLNMNKGRXX

SEQ ID NO: 18 - protein, 873 aa, Artificial Sequence
  Made in Lab - megaTAL I-OnuI variant BTK L4 V25 construct
  MOD_RES (872)..(873): Any amino acid or absent
MGSAPPKKKRKVVDLRTLGYSQQQQEKIKPKVRSTVAQHHEALVGHGFTHAHIVALSQHPAALGTVAVTYQHIITALPEATHEDIVGVGKQWSGARALEALLTDAGELRGPPLQLDTGQLVKIAKRGGVTAMEAVHASRNALTGAPLNLTPDQVVAIASNNGGKQALETVQRLLPVLCQDHGLTPDQVVAIASNIGGKQALETVQRLLPVLCQDHGLTPDQVVAIASNIGGKQALETVQRLLPVLCQDHGLTPDQVVAIASNIGGKQALETVQRLLPVLCQDHGLTPDQVVAIASNIGGKQALETVQRLLPVLCQDHGLTPDQVVAIASHDGGKQALETVQRLLPVLCQDHGLTPDQVVAIASNGGGKQALETVQRLLPVLCQDHGLTPDQVVAIASNNGGKQALETVQRLLPVLCQDHGLTPDQVVAIASNIGGKQALETVQRLLPVLCQDHGLTPDQVVAIASNNGGKQALETVQRLLPVLCQDHGLTPDQVVAIASNGGGKQALESIVAQLSRPDPALAALTNDHLVALACLGGRPAMDAVKKGLPHAPELIRRVNRRIGERTSHRVAISRVGGSSINPWILTGFADAEGWFLLVIRNSNTVKVGYRTLLSFGIELHNKDKSILENIQSTWKVGKISNSGDHYVRLTVSRFEDLKVIIDHFEKYPLITQKLGDYKLFKQAFSLMENKEHLKENGIKELVRIKAKMNWGLNDELKKAFPENISKERPLINKNIPNLKWLAGFTSGDGSFYVNLVKGKNTTRVTVQLVFQITQHIKDKNLMNSLITYLGCGYILEKNVSERSFLQFRVTKFSDINDKIIPVFQENTLIGVKLEDFEDWCKVAKLIEEKKHLTESGLDEIKKIKLNMNKGRXX

SEQ ID NO: 19 - protein, 873 aa, Artificial Sequence
  Made in Lab - megaTAL I-OnuI variant BTK EL4 V34 construct
  MOD_RES (872)..(873): Any amino acid or absent
MGSAPPKKKRKVVDLRTLGYSQQQQEKIKPKVRSTVAQHHEALVGHGFTHAHIVALSQHPAALGTVAVTYQHIITALPEATHEDIVGVGKQWSGARALEALLTDAGELRGPPLQLDTGQLVKIAKRGGVTAMEAVHASRNALTGAPLNLTPDQVVAIASNNGGKQALETVQRLLPVLCQDHGLTPDQVVAIASNIGGKQALETVQRLLPVLCQDHGLTPDQVVAIASNIGGKQALETVQRLLPVLCQDHGLTPDQVVAIASNIGGKQALETVQRLLPVLCQDHGLTPDQVVAIASNIGGKQALETVQRLLPVLCQDHGLTPDQVVAIASHDGGKQALETVQRLLPVLCQDHGLTPDQVVAIASNGGGKQALETVQRLLPVLCQDHGLTPDQVVAIASNNGGKQALETVQRLLPVLCQDHGLTPDQVVAIASNIGGKQALETVQRLLPVLCQDHGLTPDQVVAIASNNGGKQALETVQRLLPVLCQDHGLTPDQVVAIASNGGGKQALESIVAQLSRPDPALAALTNDHLVALACLGGRPAMDAVKKGLPHAPELIRRVNRRIGERTSHRVAISRVGGSSINPWILTGFADAEGWFLLVIRNSNTVKVGYRTLLSFGIELHNKDKSILENIQSTWKVGKISNSGDHYVRLTVSRFEDLKVIIDHFEKYPLITQKLGDYKLFKQAFSLMENKEHLKENGIKELVRIRAKMNWGLNDELKKAFPENISKERPLINKNIPNLKWLAGFTSGDGSFYVNLVKGKNTTRVTVQLVFQITQHIKDKNLMNSLITYLGCGYILEKNVSERSFLQFRVTKFSDIKDKIIPVFQENTLIGVKLEDFEDWCKVAKLIEEKKHLTESGLDEIKKIKLNMNKGRXX

SEQ ID NO: 20 - protein, 873 aa, Artificial Sequence
  Made in Lab - megaTAL I-OnuI variant BTK EL V42 construct
  MOD_RES (872)..(873): Any amino acid or absent
MGSAPPKKKRKVVDLRTLGYSQQQQEKIKPKVRSTVAQHHEALVGHGFTHAHIVALSQHPAALGTVAVTYQHIITALPEATHEDIVGVGKQWSGARALEALLTDAGELRGPPLQLDTGQLVKIAKRGGVTAMEAVHASRNALTGAPLNLTPDQVVAIASNNGGKQALETVQRLLPVLCQDHGLTPDQVVAIASNIGGKQALETVQRLLPVLCQDHGLTPDQVVAIASNIGGKQALETVQRLLPVLCQDHGLTPDQVVAIASNIGGKQALETVQRLLPVLCQDHGLTPDQVVAIASNIGGKQALETVQRLLPVLCQDHGLTPDQVVAIASHDGGKQALETVQRLLPVLCQDHGLTPDQVVAIASNGGGKQALETVQRLLPVLCQDHGLTPDQVVAIASNNGGKQALETVQRLLPVLCQDHGLTPDQVVAIASNIGGKQALETVQRLLPVLCQDHGLTPDQVVAIASNNGGKQALETVQRLLPVLCQDHGLTPDQVVAIASNGGGKQALESIVAQLSRPDPALAALTNDHLVALACLGGRPAMDAVKKGLPHAPELIRRVNRRIGERTSHRVAISRVGGSSINPWILTGFADAEGWFLLDIRNSNTVKVGYRTLLSFGIELHNKDKSILENIQSTWKVGKIRNSGDRYVSLTVSRFEDLKVIIDHFEKYPLITQKLGDYKLFKQAFSLMENKEHLKENGIKELVRIKAKMNWGLNDELKKAFPENISKERPLINKSIPNLKWLAGFTSGDGSFYVNLVKGKNTTRVTVQLVFQITQHIKDKYLMNSLITYLGCGYILEKNVSERSFLQFRVTKFSDINDKIIPVFQENTLIGVKLEDFEDWCKVAKLIEEKKHLTESGLDEIKKIKLNMNKGRXX

SEQ ID NO: 21 - protein, 1112 aa, Artificial Sequence
  Made in Lab - megaTAL I-OnuI variant BTK L4 V25 TREX2 fusion construct
MGSAPPKKKRKVVDLRTLGYSQQQQEKIKPKVRSTVAQHHEALVGHGFTHAHIVALSQHPAALGTVAVTYQHIITALPEATHEDIVGVGKQWSGARALEALLTDAGELRGPPLQLDTGQLVKIAKRGGVTAMEAVHASRNALTGAPLNLTPDQVVAIASNNGGKQALETVQRLLPVLCQDHGLTPDQVVAIASNIGGKQALETVQRLLPVLCQDHGLTPDQVVAIASNIGGKQALETVQRLLPVLCQDHGLTPDQVVAIASNIGGKQALETVQRLLPVLCQDHGLTPDQVVAIASNIGGKQALETVQRLLPVLCQDHGLTPDQVVAIASHDGGKQALETVQRLLPVLCQDHGLTPDQVVAIASNGGGKQALETVQRLLPVLCQDHGLTPDQVVAIASNNGGKQALETVQRLLPVLCQDHGLTPDQVVAIASNIGGKQALETVQRLLPVLCQDHGLTPDQVVAIASNNGGKQALETVQRLLPVLCQDHGLTPDQVVAIASNGGGKQALESIVAQLSRPDPALAALTNDHLVALACLGGRPAMDAVKKGLPHAPELIRRVNRRIGERTSHRVAISRVGGSSINPWILTGFADAEGWFLLVIRNSNTVKVGYRTLLSFGIELHNKDKSILENIQSTWKVGKISNSGDHYVRLTVSRFEDLKVIIDHFEKYPLITQKLGDYKLFKQAFSLMENKEHLKENGIKELVRIKAKMNWGLNDELKKAFPENISKERPLINKNIPNLKWLAGFTSGDGSFYVNLVKGKNTTRVTVQLVFQITQHIKDKNLMNSLITYLGCGYILEKNVSERSFLQFRVTKFSDINDKIIPVFQENTLIGVKLEDFEDWCKVAKLIEEKKHLTESGLDEIKKIKLNMNKGRVFASTGSEPPRAETFVFLDLEATGLPNMDPEIAEISLFAVHRSSLENPERDDSGSLVLPRVLDKLTLCMCPERPFTAKASEITGLSSESLMHCGKAGFNGAVVRTLQGFLSRQEGPICLVAHNGFDYDFPLLCTELQRLGAHLPQDTVCLDTLPALRGLDRAHSHGTRAQGRKSYSLASLFHRYFQAEPSAAHSAEGDVHTLLLIFLHRAPELLAWADEQARSWAHIEPMYVPPDGPSLEA

SEQ ID NO: 22 - protein, 1112 aa, Artificial Sequence
  Made in Lab - megaTAL I-OnuI variant BTK EL4 V34 TREX2 fusion construct
MGSAPPKKKRKVVDLRTLGYSQQQQEKIKPKVRSTVAQHHEALVGHGFTHAHIVALSQHPAALGTVAVTYQHIITALPEATHEDIVGVGKQWSGARALEALLTDAGELRGPPLQLDTGQLVKIAKRGGVTAMEAVHASRNALTGAPLNLTPDQVVAIASNNGGKQALETVQRLLPVLCQDHGLTPDQVVAIASNIGGKQALETVQRLLPVLCQDHGLTPDQVVAIASNIGGKQALETVQRLLPVLCQDHGLTPDQVVAIASNIGGKQALETVQRLLPVLCQDHGLTPDQVVAIASNIGGKQALETVQRLLPVLCQDHGLTPDQVVAIASHDGGKQALETVQRLLPVLCQDHGLTPDQVVAIASNGGGKQALETVQRLLPVLCQDHGLTPDQVVAIASNNGGKQALETVQRLLPVLCQDHGLTPDQVVAIASNIGGKQALETVQRLLPVLCQDHGLTPDQVVAIASNNGGKQALETVQRLLPVLCQDHGLTPDQVVAIASNGGGKQALESIVAQLSRPDPALAALTNDHLVALACLGGRPAMDAVKKGLPHAPELIRRVNRRIGERTSHRVAISRVGGSSINPWILTGFADAEGWFLLVIRNSNTVKVGYRTLLSFGIELHNKDKSILENIQSTWKVGKISNSGDHYVRLTVSRFEDLKVIIDHFEKYPLITQKLGDYKLFKQAFSLMENKEHLKENGIKELVRIRAKMNWGLNDELKKAFPENISKERPLINKNIPNLKWLAGFTSGDGSFYVNLVKGKNTTRVTVQLVFQITQHIKDKNLMNSLITYLGCGYILEKNVSERSFLQFRVTKFSDIKDKIIPVFQENTLIGVKLEDFEDWCKVAKLIEEKKHLTESGLDEIKKIKLNMNKGRVFASTGSEPPRAETFVFLDLEATGLPNMDPEIAEISLFAVHRSSLENPERDDSGSLVLPRVLDKLTLCMCPERPFTAKASEITGLSSESLMHCGKAGFNGAVVRTLQGFLSRQEGPICLVAHNGFDYDFPLLCTELQRLGAHLPQDTVCLDTLPALRGLDRAHSHGTRAQGRKSYSLASLFHRYFQAEPSAAHSAEGDVHTLLLIFLHRAPELLAWADEQARSWAHIEPMYVPPDGPSLEA

SEQ ID NO: 23 - protein, 1112 aa, Artificial Sequence
  Made in Lab - megaTAL I-OnuI variant BTK EL V42 TREX2 fusion construct
MGSAPPKKKRKVVDLRTLGYSQQQQEKIKPKVRSTVAQHHEALVGHGFTHAHIVALSQHPAALGTVAVTYQHIITALPEATHEDIVGVGKQWSGARALEALLTDAGELRGPPLQLDTGQLVKIAKRGGVTAMEAVHASRNALTGAPLNLTPDQVVAIASNNGGKQALETVQRLLPVLCQDHGLTPDQVVAIASNIGGKQALETVQRLLPVLCQDHGLTPDQVVAIASNIGGKQALETVQRLLPVLCQDHGLTPDQVVAIASNIGGKQALETVQRLLPVLCQDHGLTPDQVVAIASNIGGKQALETVQRLLPVLCQDHGLTPDQVVAIASHDGGKQALETVQRLLPVLCQDHGLTPDQVVAIASNGGGKQALETVQRLLPVLCQDHGLTPDQVVAIASNNGGKQALETVQRLLPVLCQDHGLTPDQVVAIASNIGGKQALETVQRLLPVLCQDHGLTPDQVVAIASNNGGKQALETVQRLLPVLCQDHGLTPDQVVAIASNGGGKQALESIVAQLSRPDPALAALTNDHLVALACLGGRPAMDAVKKGLPHAPELIRRVNRRIGERTSHRVAISRVGGSSINPWILTGFADAEGWFLLDIRNSNTVKVGYRTLLSFGIELHNKDKSILENIQSTWKVGKIRNSGDRYVSLTVSRFEDLKVIIDHFEKYPLITQKLGDYKLFKQAFSLMENKEHLKENGIKELVRIKAKMNWGLNDELKKAFPENISKERPLINKSIPNLKWLAGFTSGDGSFYVNLVKGKNTTRVTVQLVFQITQHIKDKYLMNSLITYLGCGYILEKNVSERSFLQFRVTKFSDINDKIIPVFQENTLIGVKLEDFEDWCKVAKLIEEKKHLTESGLDEIKKIKLNMNKGRVFASTGSEPPRAETFVFLDLEATGLPNMDPEIAEISLFAVHRSSLENPERDDSGSLVLPRVLDKLTLCMCPERPFTAKASEITGLSSESLMHCGKAGFNGAVVRTLQGFLSRQEGPICLVAHNGFDYDFPLLCTELQRLGAHLPQDTVCLDTLPALRGLDRAHSHGTRAQGRKSYSLASLFHRYFQAEPSAAHSAEGDVHTLLLIFLHRAPELLAWADEQARSWAHIEPMYVPPDGPSLEA

SEQ ID NO: 24 - DNA, 22 bp, Homo sapiens
atatcaaggacttggccttaga

SEQ ID NO: 25 - DNA, 11 bp, Homo sapiens
gaaaactgagt

SEQ ID NO: 26 - DNA, 39 bp, Homo sapiens
gaaaactgagtttcaagatatcaaggacttggccttaga

SEQ ID NO: 27 - DNA, 2613 bp, Artificial Sequence
  Made in Lab - I-OnuI variant BTK L4 V25 mRNA
augggauccgcgccacctaagaagaaacgcaaagtcgtggatctacgcacgctcggctacagtcagcagcagcaagagaagatcaaaccgaaggtgcgttcgacagtggcgcagcaccacgaggcactggtgggccatgggtttacacacgcgcacatcgttgcgctcagccaacacccggcagcgttagggaccgtcgctgtcacgtatcagcacataatcacggcgttgccagaggcgacacacgaagacatcgttggcgtcggcaaacagtggtccggcgcacgcgccctggaggccttgctcacggatgcgggggagttgagaggtccgccgttacagttggacacaggccaacttgtgaagattgcaaaacgtggcggcgtgaccgcaatggaggcagtgcatgcatcgcgcaatgcactgacgggtgcccccctgaacctgaccccggaccaagtggtggctatcgccagcaacaatggcggcaagcaagcgctcgaaacggtgcagcggctgttgccggtgctgtgccaggaccatggcctgaccccggaccaagtggtggctatcgccagcaacattggcggcaagcaagcgctcgaaacggtgcagcggctgttgccggtgctgtgccaggaccatggcctgactccggaccaagtggtggctatcgccagcaacattggcggcaagcaagcgctcgaaacggtgcagcggctgttgccggtgctgtgccaggaccatggcctgactccggaccaagtggtggctatcgccagcaacattggcggcaagcaagcgctcgaaacggtgcagcggctgttgccggtgctgtgccaggaccatggcctgaccccggaccaagtggtggctatcgccagcaacattggcggcaagcaagcgctcgaaacggtgcagcggctgttgccggtgctgtgccaggaccatggcctgaccccggaccaagtggtggctatcgccagccacgatggcggcaagcaagcgctcgaaacggtgcagcggctgttgccggtgctgtgccaggaccatggcctgaccccggaccaagtggtggctatcgccagcaacggtggcggcaagcaagcgctcgaaacggtgcagcggctgttgccggtgctgtgccaggaccatggcctgaccccggaccaagtggtggctatcgccagcaacaatggcggcaagcaagcgctcgaaacggtgcagcggctgttgccggtgctgtgccaggaccatggcctgaccccggaccaagtggtggctatcgccagcaacattggcggcaagcaagcgctggaaacggtgcagcggctgttgccggtgctgtgccaggaccatggcctgaccccggaccaagtggtggctatcgccagcaacaatggcggcaagcaagcgctcgaaacggtgcagcggctgttgccggtgctgtgccaggaccatggcctgaccccggaccaagtggtggctatcgccagcaacggtggcggcaagcaagcgctcgaaagcattgtggcccagctgagccggcctgatccggcgttggccgcgttgaccaacgaccacctcgtcgccttggcctgcctcggcggacgtcctgccatggatgcagtgaaaaagggattgccgcacgcgccggaattgatcagaagagtcaatcgccgtattggcgaacgcacgtcccatcgcgttgcgatatctagagtgggaggaagctccatcaacccatggattctgactggtttcgctgatgccgaaggatggttcttgctagttatccggaactcaaacacagttaaggtggggtacaggactttgctgagcttcggtatcgaactgcacaacaaggacaaatcgattctggagaatatccagtcgacttggaaggtcggcaagatcagtaacagcggcgaccactatgtccgtctgacagtctctcgtttcgaagatttgaaagtgattatcgaccacttcgagaaatatccgctgattacccagaaattgggcgattacaagctgtttaaacaggcattcagcctcatggagaacaaagaacatctcaaggagaatgggattaaggagctcgtacgaatcaaagctaagatgaattggggtctcaatgacgaattgaaaaaagcatttccagagaacattagcaaagagcgcccccttatcaataagaacattccgaatctcaaatggctggctggattcacatctggtgacggctctttctacgtgaatctagttaagggaaaaaacacgacgagagtaactgtgcagctggttttccaaatcacgcagcacatcaaagacaagaacctgatgaattcattgataacatacctaggctgtggttatatcctagagaagaacgtatctgagagaagttttctccagttcagagtaactaaattcagcgatatcaacgacaagatcattccggtattccaggaaaatactctgattggcgtcaaactcgaggactttgaagattggtgcaaggttgccaaattgatcgaagagaagaaacacctgaccgaatccggtttggatgagattaagaaaatcaagctgaacatgaacaaaggtcgt

SEQ ID NO: 28 - DNA, 2613 bp, Artificial Sequence
  Made in Lab - I-OnuI variant BTK EL4 V34 mRNA
augggauccgcgccacctaagaagaaacgcaaagtcgtggatctacgcacgctcggctacagtcagcagcagcaagagaagatcaaaccgaaggtgcgttcgacagtggcgcagcaccacgaggcactggtgggccatgggtttacacacgcgcacatcgttgcgctcagccaacacccggcagcgttagggaccgtcgctgtcacgtatcagcacataatcacggcgttgccagaggcgacacacgaagacatcgttggcgtcggcaaacagtggtccggcgcacgcgccctggaggccttgctcacggatgcgggggagttgagaggtccgccgttacagttggacacaggccaacttgtgaagattgcaaaacgtggcggcgtgaccgcaatggaggcagtgcatgcatcgcgcaatgcactgacgggtgcccccctgaacctgaccccggaccaagtggtggctatcgccagcaacaatggcggcaagcaagcgctcgaaacggtgcagcggctgttgccggtgctgtgccaggaccatggcctgaccccggaccaagtggtggctatcgccagcaacattggcggcaagcaagcgctcgaaacggtgcagcggctgttgccggtgctgtgccaggaccatggcctgactccggaccaagtggtggctatcgccagcaacattggcggcaagcaagcgctcgaaacggtgcagcggctgttgccggtgctgtgccaggaccatggcctgactccggaccaagtggtggctatcgccagcaacattggcggcaagcaagcgctcgaaacggtgcagcggctgttgccggtgctgtgccaggaccatggcctgaccccggaccaagtggtggctatcgccagcaacattggcggcaagcaagcgctcgaaacggtgcagcggctgttgccggtgctgtgccaggaccatggcctgaccccggaccaagtggtggctatcgccagccacgatggcggcaagcaagcgctcgaaacggtgcagcggctgttgccggtgctgtgccaggaccatggcctgaccccggaccaagtggtggctatcgccagcaacggtggcggcaagcaagcgctcgaaacggtgcagcggctgttgccggtgctgtgccaggaccatggcctgaccccggaccaagtggtggctatcgccagcaacaatggcggcaagcaagcgctcgaaacggtgcagcggctgttgccggtgctgtgccaggaccatggcctgaccccggaccaagtggtggctatcgccagcaacattggcggcaagcaagcgctggaaacggtgcagcggctgttgccggtgctgtgccaggaccatggcctgaccccggaccaagtggtggctatcgccagcaacaatggcggcaagcaagcgctcgaaacggtgcagcggctgttgccggtgctgtgccaggaccatggcctgaccccggaccaagtggtggctatcgccagcaacggtggcggcaagcaagcgctcgaaagcattgtggcccagctgagccggcctgatccggcgttggccgcgttgaccaacgaccacctcgtcgccttggcctgcctcggcggacgtcctgccatggatgcagtgaaaaagggattgccgcacgcgccggaattgatcagaagagtcaatcgccgtattggcgaacgcacgtcccatcgcgttgcgatatctagagtgggaggaagctccatcaacccatggattctgactggtttcgctgatgccgaaggatggttcttgctagttatccggaactcaaacacagttaaggtggggtacaggactttgctgagcttcggtatcgaactgcacaacaaggacaaatcgattctggagaatatccagtcgacttggaaggtcggcaagatcagtaacagcggcgaccactatgtccgtctgacagtctctcgtttcgaagatttgaaagtgattatcgaccacttcgagaaatatccgctgattacccagaaattgggcgattacaagctgtttaaacaggcattcagcctcatggagaacaaagaacatctcaaggagaatgggattaaggagctcgtacgaatcagagctaagatgaattggggtctcaatgacgaattgaaaaaagcatttccagagaacattagcaaagagcgcccccttatcaataagaacattccgaatctcaaatggctggctggattcacatctggtgacggctctttctacgtgaatctagttaagggaaaaaacacgacgagagtaactgtgcagctggttttccaaatcacgcagcacatcaaagacaagaacctgatgaattcattgataacatacctaggctgtggttatatcctagagaagaacgtatctgagagaagttttctccagttcagagtaactaaattcagcgatatcaaggacaagatcattccggtattccaggaaaatactctgattggcgtcaaactcgaggactttgaagattggtgcaaggttgccaaattgatcgaagagaagaaacacctgaccgaatccggtttggatgagattaagaaaatcaagctgaacatgaacaaaggtcgt

SEQ ID NO: 29 - DNA, 2613 bp, Artificial Sequence
  Made in Lab - I-OnuI variant BTK EL V42 mRNA
augggauccgcgccacctaagaagaaacgcaaagtcgtggatctacgcacgctcggctacagtcagcagcagcaagagaagatcaaaccgaaggtgcgttcgacagtggcgcagcaccacgaggcactggtgggccatgggtttacacacgcgcacatcgttgcgctcagccaacacccggcagcgttagggaccgtcgctgtcacgtatcagcacataatcacggcgttgccagaggcgacacacgaagacatcgttggcgtcggcaaacagtggtccggcgcacgcgccctggaggccttgctcacggatgcgggggagttgagaggtccgccgttacagttggacacaggccaacttgtgaagattgcaaaacgtggcggcgtgaccgcaatggaggcagtgcatgcatcgcgcaatgcactgacgggtgcccccctgaacctgaccccggaccaagtggtggctatcgccagcaacaatggcggcaagcaagcgctcgaaacggtgcagcggctgttgccggtgctgtgccaggaccatggcctgaccccggaccaagtggtggctatcgccagcaacattggcggcaagcaagcgctcgaaacggtgcagcggctgttgccggtgctgtgccaggaccatggcctgactccggaccaagtggtggctatcgccagcaacattggcggcaagcaagcgctcgaaacggtgcagcggctgttgccggtgctgtgccaggaccatggcctgactccggaccaagtggtggctatcgccagcaacattggcggcaagcaagcgctcgaaacggtgcagcggctgttgccggtgctgtgccaggaccatggcctgaccccggaccaagtggtggctatcgccagcaacattggcggcaagcaagcgctcgaaacggtgcagcggctgttgccggtgctgtgccaggaccatggcctgaccccggaccaagtggtggctatcgccagccacgatggcggcaagcaagcgctcgaaacggtgcagcggctgttgccggtgctgtgccaggaccatggcctgaccccggaccaagtggtggctatcgccagcaacggtggcggcaagcaagcgctcgaaacggtgcagcggctgttgccggtgctgtgccaggaccatggcctgaccccggaccaagtggtggctatcgccagcaacaatggcggcaagcaagcgctcgaaacggtgcagcggctgttgccggtgctgtgccaggaccatggcctgaccccggaccaagtggtggctatcgccagcaacattggcggcaagcaagcgctggaaacggtgcagcggctgttgccggtgctgtgccaggaccatggcctgaccccggaccaagtggtggctatcgccagcaacaatggcggcaagcaagcgctcgaaacggtgcagcggctgttgccggtgctgtgccaggaccatggcctgaccccggaccaagtggtggctatcgccagcaacggtggcggcaagcaagcgctcgaaagcattgtggcccagctgagccggcctgatccggcgttggccgcgttgaccaacgaccacctcgtcgccttggcctgcctcggcggacgtcctgccatggatgcagtgaaaaagggattgccgcacgcgccggaattgatcagaagagtcaatcgccgtattggcgaacgcacgtcccatcgcgttgcgatatctagagtgggaggaagctccatcaacccatggattctgactggtttcgctgatgccgaaggatggttcctgctagatatccggaactcaaacacagttaaggtggggtacaggactttgctgagcttcggtatcgaactgcacaacaaggacaaatcgattctggagaatatccagtcgacttggaaggtcggcaagatcagaaacagcggcgaccgctatgtcagtctgaccgtctctcgtttcgaagatttgaaagtgattatcgaccacttcgagaaatatccgctgattacccagaaattgggcgattacaagctgtttaaacaggcattcagcctcatggagaacaaagaacatctcaaggagaatgggattaaggagctcgtacgaatcaaagctaagatgaattggggtctcaatgacgaattgaaaaaagcatttccagagaacattagcaaagagcgcccccttatcaataagagcattccgaatctcaaatggctggctggattcacatctggtgacggctctttctacgtgaatctagttaagggaaaaaacacgacgagagtaactgtgcagctggttttccaaatcacgcagcacatcaaagacaagtacctgatgaattcattgataacatacctaggctgtggttatatcctagagaagaacgtatctgagagaagttttctccagttcagagtaactaaattcagcgatatcaacgacaagatcattccggtattccaggaaaatactctgattggcgtcaaactcgaggactttgaagattggtgcaaggttgccaaattgatcgaagagaagaaacacctgaccgaatccggtttggatgagattaagaaaatcaagctgaacatgaacaaaggtcgt

SEQ ID NO: 30 - RNA, 711 nt, Mus musculus
augucugagccaccucgggcugagaccuuuguauuccuggaccuagaagccacugggcucccaaacauggacccugagauugcagagauaucccuuuuugcuguucaccgcucuucccuggagaacccagaacgggaugauucugguuccuuggugcugccccguguucuggacaagcucacacugugcaugugcccggagcgccccuuuacugccaaggccagugagauuacugguuugagcagcgaaagccugaugcacugcgggaaggcugguuucaauggcgcugugguaaggacacugcagggcuuccuaagccgccaggagggccccaucugccuuguggcccacaauggcuucgauuaugacuucccacugcugugcacggagcuacaacgucugggugcccaucugccccaagacacugucugccuggacacacugccugcauugcggggccuggaccgugcucacagccacggcaccagggcucaaggccgcaaaagcuacagccuggccagucucuuccaccgcuacuuccaggcugaacccagugcugcccauucagcagaaggugaugugcacacccugcuucugaucuuccugcaucgugcuccugagcugcucgccugggcagaugagcaggcccgcagcugggcucauauugagcccauguacgugccaccugaugguccaagccucgaagccuga

SEQ ID NO: 31 - protein, 236 aa, Mus musculus
MSEPPRAETFVFLDLEATGLPNMDPEIAEISLFAVHRSSLENPERDDSGSLVLPRVLDKLTLCMCPERPFTAKASEITGLSSESLMHCGKAGFNGAVVRTLQGFLSRQEGPICLVAHNGFDYDFPLLCTELQRLGAHLPQDTVCLDTLPALRGLDRAHSHGTRAQGRKSYSLASLFHRYFQAEPSAAHSAEGDVHTLLLIFLHRAPELLAWADEQARSWAHIEPMYVPPDGPSLEA

SEQ ID NO: 32 - protein, 659 aa, Homo sapiens
MAAVILESIFLKRSQQKKKTSPLNFKKRLFLLTVHKLSYYEYDFERGRRGSKKGSIDVEKITCVETVVPEKNPPPERQIPRRGEESSEMEQISIIERFPYPFQVVYDEGPLYVFSPTEELRKRWIHQLKNVIRYNSDLVQKYHPCFWIDGQYLCCSQTAKNAMGCQILENRNGSLKPGSSHRKTKKPLPPTPEEDQILKKPLPPEPAAAPVSTSELKKVVALYDYMPMNANDLQLRKGDEYFILEESNLPWWRARDKNGQEGYIPSNYVTEAEDSIEMYEWYSKHMTRSQAEQLLKQEGKEGGFIVRDSSKAGKYTVSVFAKSTGDPQGVIRHYVVCSTPQSQYYLAEKHLFSTIPELINYHQHNSAGLISRLKYPVSQQNKNAPSTAGLGYGSWEIDPKDLTFLKELGTGQFGVVKYGKWRGQYDVAIKMIKEGSMSEDEFIEEAKVMMNLSHEKLVQLYGVCTKQRPIFIITEYMANGCLLNYLREMRHRFQTQQLLEMCKDVCEAMEYLESKQFLHRDLAARNCLVNDQGVVKVSDFGLSRYVLDDEYTSSVGSKFPVRWSPPEVLMYSKFSSKSDIWAFGVLMWEIYSLGKMPYERFTNSETAEHIAQGLRLYRPHLASEKVYTIMYSCWHEKADERPTFKILLSNILDVMDEES

SEQ ID NO: 33 - DNA, 7174 bp, Artificial Sequence
  Made in Lab - AAV plasmid construct, pAAV-BTK mTAL MND.GFP.PA
cagctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacacgcgtatgaacagaggtggcatctatatcagtaagacagttgcatcacttttgcatgatgctgtctaaaagaactaatttaagctaaatggggaaaaggtcagaaaacaacaactaccccccccccaccaaaacccaccaaaaaaaattatgttttcaactttagaacaaatcttctatcctttgtagctcagtcagtgggtgtgggcaaaatcagttgggcagcagttagtgtgtgtccagaactgcaggtgcagcctccatatccttattagttcccttggttacagaccccagtgggacaatgtttgaaaaattatattcaccgtctaggaaattgggaactgaaagtccaatatctgcctcagtggagttctggcacctgcattatcccttctgggtatatcaagatcaacagctgcacagatacttttgcttttcacagattctacacatatcatataaaggtgaatagtgtaaagctacctctacaccttaccaagcacacaggtgcgtgccatttaacatctagagcattccattgccttatacaagaactcagtttatatgagctcacaacatcgaaccaatccccccccaattcagtgtgcatccattatacctgaaacctgacagagctgggggctgtgggaggaggttggtaggaagaaattattttgtgagctgtgcacatttttgttccatttgaaactaggtagctaggctgagggggaaccaagagggatgaggattaatgtcctgggtcctcaggaactttcattatcaacagcacacaggtgaactccagaaagaagaagctatggccgcagtgattctggagagcatctttctgaagcgatcccaacagaaaaagaaaacatcacctctaaacttcaagaagcgcctgtttctcttgaccgtgcacaaactctcctactatgagtatgactttgaacgtggggtaagtttctcgactatgaaaactgagtttcaagatatcaaggacgaacagagaaacaggagaatatgggccaaacaggatatctgtggtaagcagttcctgccccggctcagggccaagaacagttggaacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaatgaccctgtgccttatttgaactaaccaatcagttcgcttctcgcttctgttcgcgcgcttctgctccccgagctctatataagcagagctcgtttagtgaaccgtcagatcgcctggagacgccatccacgctgttttgacttccatagaaggatctcgaggccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaactagtgtcgactgctttatttgtgaaatttgtgatgctattgctttatttgtaaccattataagctgcaataaacaagttaacaacaacaattgcattcattttatgtttcaggttcagggggaggtgtgggaggttttttaaattggccttagatctttcttggggaagaggtaaattttcgttggtaggaggaggggagtagaatggacctaagttctttcaaattcagcaaaatatttcctagcctataactagctaaagccggaaagtcaaaggtcctaagaagccacaaggaaaatattaccatggaatcttggaattgatgagcactcattaaatgattgttgaaaatgaaatcgaagagttggaaattgcttccttacttcctatgaggaaggtacatacagtcattcactcttccatggtatttgccctccatttggtagtcatagatttatagatctggaaggatttttttttcttcccccacatgacaggtcctggtgccacctcactttgttgaatgattagataacaaaatctaatcatctggttgcttaatccctcttaatctttctccattttcttcctcattctacttctcagagaagaggcagtaagaagggttcaatagatgttgagaagatcacttgtgttgaaacagtggttcctgaaaaaaatcctcctccagaaagacagattccggtaagaagagaccaatgtctgagatggggaacagcagatttgaagaaatttgcaacatttaaattctctgtaaatagactggtgatgctgtgcaacgtggaacacggtcaagtttcctttaaaaattcttcactctaccatattggttataaagaatcttagcttctttccttcatattcagaacatctcactaaacatggaaaatttgttaacacaaacttttaaatgatgctatatctagttttcaaactggtcagagatcattgattttattccctcagttctctcaggatcagatttagaggcttaagtaagtctgaatgtcataatcctagggctctgagtcacatgatatcctttaataccttactatttattctcttctcactttccggagcgagagatctagagtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgccagctggcgtaatagcgaagaggcccgcaccgatcgcccttcccaacagttgcgcagcctgaatggcgaatggcgattccgttgcaatggctggcggtaatattgttctggatattaccagcaaggccgatagtttgagttcttctactcaggcaagtgatgttattactaatcaaagaagtattgcgacaacggttaatttgcgtgatggacagactcttttactcggtggcctcactgattataaaaacacttctcaggattctggcgtaccgttcctgtctaaaatccctttaatcggcctcctgtttagctcccgctctgattctaacgaggaaagcacgttatacgtgctcgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttaaatatttgcttatacaatcttcctgtttttggggcttttctgattatcaaccggggtacatatgattgacatgctagttttacgattaccgttcatcgattctcttgtttgctccagactctcaggcaatgacctgatagcctttgtagagacctctcaaaaatagctaccctctccggcatgaatttatcagctagaacggttgaatatcatattgatggtgatttgactgtctccggcctttctcacccgtttgaatctttacctacacattactcaggcattgcatttaaaatatatgagggttctaaaaatttttatccttgcgttgaaataaaggcttctcccgcaaaagtattacagggtcataatgtttttggtacaaccgatttagctttatgctctgaggctttattgcttaattttgctaattctttgccttgcctgtatgatttattggatgttggaatcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatg

SEQ ID NO: 34 - DNA, 7308 bp, Artificial Sequence
  Made in Lab - AAV plasmid construct, pAAV.BTK.1.0.1183jxn.MND.GFP.SV40pA
cagctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacacgcgttctaaaagaactaatttaagctaaatggggaaaaggtcagaaaacaacaactaccccccccccaccaaaacccaccaaaaaaaattatgttttcaagggaactttatttgtctttctgtgtttcagttacctaaattgaatccttctggagtattgtaggtttggggaggctaaataagttgtgtttcataaatgaacagaggtggcatctatatcagtaagacagttgcatcacttttgcatgatgctgtctaaaagaactaatttaagctaaatggggaaaaggtcagaaaacaacaactaccccccccccaccaaaacccaccaaaaaaaattatgttttcaactttagaacaaatcttctatcctttgtagctcagtcagtgggtgtgggcaaaatcagttgggcagcagttagtgtgtgtccagaactgcaggtgcagcctccatatccttattagttcccttggttacagaccccagtgggacaatgtttgaaaaattatattcaccgtctaggaaattgggaactgaaagtccaatatctgcctcagtggagttctggcacctgcattatcccttctgggtatatcaagatcaacagctgcacagatacttttgcttttcacagattctacacatatcatataaaggtgaatagtgtaaagctacctctacaccttaccaagcacacaggtgcgtgccatttaacatctagagcattccattgccttatacaagaactcagtttatatgagctcacaacatcgaaccaatccccccccaattcagtgtgcatccattatacctgaaacctgacagagctgggggctgtgggaggaggttggtaggaagaaattattttgtgagctgtgcacatttttgttccatttgaaactaggtagctaggctgagggggaaccaagagggatgaggattaatgtcctgggtcctcaggaactttcattatcaacagcacacaggtgaactccagaaagaagaagctatggccgcagtgattctggagagcatctttctgaagcgatcccgaacagagaaacaggagaatatgggccaaacaggatatctgtggtaagcagttcctgccccggctcagggccaagaacagttggaacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaatgaccctgtgccttatttgaactaaccaatcagttcgcttctcgcttctgttcgcgcgcttctgctccccgagctctatataagcagagctcgtttagtgaaccgtcagatcgcctggagacgccatccacgctgttttgacttccatagaaggatctcgaggccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaactagtgtcgactgctttatttgtgaaatttgtgatgctattgctttatttgtaaccattataagctgcaataaacaagttaacaacaacaattgcattcattttatgtttcaggttcagggggaggtgtgggaggttttttaaaagctaaagccggaaagtcaaaggtcctaagaagccacaaggaaaatattaccatggaatcttggaattgatgagcactcattaaatgattgttgaaaatgaaatcgaagagttggaaattgcttccttacttcctatgaggaaggtacatacagtcattcactcttccatggtatttgccctccatttggtagtcatagatttatagatctggaaggatttttttttcttcccccacatgacaggtcctggtgccacctcactttgttgaatgattagataacaaaatctaatcatctggttgcttaatccctcttaatctttctccattttcttcctcattctacttctcagagaagaggcagtaagaagggttcaatagatgttgagaagatcacttgtgttgaaacagtggttcctgaaaaaaatcctcctccagaaagacagattccggtaagaagagaccaatgtctgagatggggaacagcagatttgaagaaatttgcaacatttaaattctctgtaaatagactggtgatgctgtgcaacgtggaacacggtcaagtttcctttaaaaattcttcactctaccatattggttataaagaatcttagcttctttccttcatattcagaacatctcactaaacatggaaaatttgttaacacaaacttttaaatgatgctatatctagttttcaaactggtcagagatcattgattttattccctcagttctctcaggatcagatttagaggcttaagtaagtctgaatgtcataatcctagggctctgagtcacatgatatcctttaataccttactatttattctcttctcactttccggagcgagagacataaaacctactgatttttgagttcacttttaaaaaatatatatcaatttcagtattttctttttttcttttttttttctttttttagacagagtctcgctctgttgcccaggctggaatgcactggtgccatcttggctcactgcaaccttcacctcccgggttcaagcaattctcatgcctcagcctcccaagtctagagtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgccagctggcgtaatagcgaagaggcccgcaccgatcgcccttcccaacagttgcgcagcctgaatggcgaatggcgattccgttgcaatggctggcggtaatattgttctggatattaccagcaaggccgatagtttgagttcttctactcaggcaagtgatgttattactaatcaaagaagtattgcgacaacggttaatttgcgtgatggacagactcttttactcggtggcctcactgattataaaaacacttctcaggattctggcgtaccgttcctgtctaaaatccctttaatcggcctcctgtttagctcccgctctgattctaacgaggaaagcacgttatacgtgctcgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttaaatatttgcttatacaatcttcctgtttttggggcttttctgattatcaaccggggtacatatgattgacatgctagttttacgattaccgttcatcgattctcttgtttgctccagactctcaggcaatgacctgatagcctttgtagagacctctcaaaaatagctaccctctccggcatgaatttatcagctagaacggttgaatatcatattgatggtgatttgactgtctccggcctttctcacccgtttgaatctttacctacacattactcaggcattgcatttaaaatatatgagggttctaaaaatttttatccttgcgttgaaataaaggcttctcccgcaaaagtattacagggtcataatgtttttggtacaaccgatttagctttatgctctgaggctttattgcttaattttgctaattctttgccttgcctgtatgatttattggatgttggaatcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatg

SEQ ID NO: 35 - DNA, 5718 bp, Artificial Sequence
  Made in Lab - AAV plasmid construct, pAAV BTK.mTAL.ATG.coBTKV2.WPRE3.SV40pA
cagctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacacgcgtattcaccgtctaggaaattgggaactgaaagtccaatatctgcctcagtggagttctggcacctgcattatcccttctgggtatatcaagatcaacagctgcacagatacttttgcttttcacagattctacacatatcatataaaggtgaatagtgtaaagctacctctacaccttaccaagcacacaggtgcgtgccatttaacatctagagcattccattgccttatacaagaactcagtttatatgagctcacaacatcgaaccaatccccccccaattcagtgtgcatccattatacctgaaacctgacagagctgggggctgtgggaggaggttggtaggaagaaattattttgtgagctgtgcacatttttgttccatttgaaactaggtagctaggctgagggggaaccaagagggatgaggattaatgtcctgggtcctcaggaactttcattatcaacagcacacaggtgaactccagaaagaagaagctatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagggatccggtgagggcagaggaagtcttctaacatgcggtgacgtggaggagaatccgggccccagaagaggcagtaagaagggttcaatagatgttgagaagatcacttgtgttgaaacagtggttcctgaaaaaaatcctcctccagaaagacagattccggtaagaagagaccaatgtctgagatggggaacagcagatttgaagaaatttgcaacatttaaattctctgtaaatagactggtgatgctgtgcaacgtggaacacggtcaagtttcctttaaaaattcttcactctaccatattggttataaagaatcttagcttctttccttcatattcagaacatctcactaaacatggaaaatttgttaacacaaacttttaaatgatgctatatctagttttcaaactggtcagagatcattgattttattccctcagttctctcaggatcagatttagaggcttaagtaagtctgaatgtcataatcctagggctctgagtcacatgatatcctttaataccttactatttattctcttctcactttccggagcgatctagagtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgccagctggcgtaatagcgaagaggcccgcaccgatcgcccttcccaacagttgcgcagcctgaatggcgaatggcgattccgttgcaatggctggcggtaatattgttctggatattaccagcaaggccgatagtttgagttcttctactcaggcaagtgatgttattactaatcaaagaagtattgcgacaacggttaatttgcgtgatggacagactcttttactcggtggcctcactgattataaaaacacttctcaggattctggcgtaccgttcctgtctaaaatccctttaatcggcctcctgtttagctcccgctctgattctaacgaggaaagcacgttatacgtgctcgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttaaatatttgcttatacaatcttcctgtttttggggcttttctgattatcaaccggggtacatatgattgacatgctagttttacgattaccgttcatcgattctcttgtttgctccagactctcaggcaatgacctgatagcctttgtagagacctctcaaaaatagctaccctctccggcatgaatttatcagctagaacggttgaatatcatattgatggtgatttgactgtctccggcctttctcacccgtttgaatctttacctacacattactcaggcattgcatttaaaatatatgagggttctaaaaatttttatccttgcgttgaaataaaggcttctcccgcaaaagtattacagggtcataatgtttttggtacaaccgatttagctttatgctctgaggctttattgcttaattttgctaattctttgccttgcctgtatgatttattggatgttggaatcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatg

SEQ ID NO: 36 - protein, 3 aa, Artificial Sequence
  Exemplary linker sequence
GGG

SEQ ID NO: 37 - protein, 5 aa, Artificial Sequence
  Exemplary linker sequence
DGGGS

SEQ ID NO: 38 - protein, 5 aa, Artificial Sequence
  Exemplary linker sequence
TGEKP

SEQ ID NO: 39 - protein, 4 aa, Artificial Sequence
  Exemplary linker sequence
GGRR

SEQ ID NO: 40 - protein, 5 aa, Artificial Sequence
  Exemplary linker sequence
GGGGS

SEQ ID NO: 41 - protein, 14 aa, Artificial Sequence
  Exemplary linker sequence
EGKSSGSGSESKVD

SEQ ID NO: 42 - protein, 18 aa, Artificial Sequence
  Exemplary linker sequence
KESGSVSSEQLAQFRSLD

SEQ ID NO: 43 - protein, 8 aa, Artificial Sequence
  Exemplary linker sequence
GGRRGGGS

SEQ ID NO: 44 - protein, 9 aa, Artificial Sequence
  Exemplary linker sequence
LRQRDGERP

SEQ ID NO: 45 - protein, 12 aa, Artificial Sequence
  Exemplary linker sequence
LRQKDGGGSERP

SEQ ID NO: 46 - protein, 16 aa, Artificial Sequence
  Exemplary linker sequence
LRQKDGGGSGGGSERP

SEQ ID NO: 47 - protein, 7 aa, Artificial Sequence
  Cleavage sequence by TEV protease
  misc_feature (2)..(3): Xaa is any amino acid
  misc_feature (5)..(5): Xaa is any amino acid
  MISC_FEATURE (7)..(7): Xaa = Gly or Ser
EXXYXQX

SEQ ID NO: 48 - protein, 7 aa, Artificial Sequence
  Cleavage sequence by TEV protease
ENLYFQG

SEQ ID NO: 49 - protein, 7 aa, Artificial Sequence
  Cleavage sequence by TEV protease
ENLYFQS

SEQ ID NO: 50 - protein, 22 aa, Artificial Sequence
  Self-cleaving polypeptide comprising 2A site
GSGATNFSLLKQAGDVEENPGP

SEQ ID NO: 51 - protein, 19 aa, Artificial Sequence
  Self-cleaving polypeptide comprising 2A site
ATNFSLLKQAGDVEENPGP

SEQ ID NO: 52 - protein, 14 aa, Artificial Sequence
  Self-cleaving polypeptide comprising 2A site
LLKQAGDVEENPGP

SEQ ID NO: 53 - protein, 21 aa, Artificial Sequence
  Self-cleaving polypeptide comprising 2A site
GSGEGRGSLLTCGDVEENPGP

SEQ ID NO: 54 - protein, 18 aa, Artificial Sequence
  Self-cleaving polypeptide comprising 2A site
EGRGSLLTCGDVEENPGP

SEQ ID NO: 55 - protein, 13 aa, Artificial Sequence
  Self-cleaving polypeptide comprising 2A site
LLTCGDVEENPGP

SEQ ID NO: 56 - protein, 23 aa, Artificial Sequence
  Self-cleaving polypeptide comprising 2A site
GSGQCTNYALLKLAGDVESNPGP

SEQ ID NO: 57 - protein, 20 aa, Artificial Sequence
  Self-cleaving polypeptide comprising 2A site
QCTNYALLKLAGDVESNPGP

SEQ ID NO: 58 - protein, 14 aa, Artificial Sequence
  Self-cleaving polypeptide comprising 2A site
LLKLAGDVESNPGP

SEQ ID NO: 59 - protein, 25 aa, Artificial Sequence
  Self-cleaving polypeptide comprising 2A site
GSGVKQTLNFDLLKLAGDVESNPGP

SEQ ID NO: 60 - protein, 22 aa, Artificial Sequence
  Self-cleaving polypeptide comprising 2A site
VKQTLNFDLLKLAGDVESNPGP

SEQ ID NO: 61 - protein, 14 aa, Artificial Sequence
  Self-cleaving polypeptide comprising 2A site
LLKLAGDVESNPGP

SEQ ID NO: 62 - protein, 19 aa, Artificial Sequence
  Self-cleaving polypeptide comprising 2A site
LLNFDLLKLAGDVESNPGP

SEQ ID NO: 63 - protein, 19 aa, Artificial Sequence
  Self-cleaving polypeptide comprising 2A site
TLNFDLLKLAGDVESNPGP

SEQ ID NO: 64 - protein, 14 aa, Artificial Sequence
  Self-cleaving polypeptide comprising 2A site
LLKLAGDVESNPGP

SEQ ID NO: 65 - protein, 17 aa, Artificial Sequence
  Self-cleaving polypeptide comprising 2A site
NFDLLKLAGDVESNPGP

SEQ ID NO: 66 - protein, 20 aa, Artificial Sequence
  Self-cleaving polypeptide comprising 2A site
QLLNFDLLKLAGDVESNPGP

SEQ ID NO: 67 - protein, 24 aa, Artificial Sequence
  Self-cleaving polypeptide comprising 2A site
APVKQTLNFDLLKLAGDVESNPGP

SEQ ID NO: 68 - protein, 40 aa, Artificial Sequence
  Self-cleaving polypeptide comprising 2A site
VTELLYRMKRAETYCPRPLLAIHPTEARHKQKIVAPVKQT

SEQ ID NO: 69 - protein, 18 aa, Artificial Sequence
  Self-cleaving polypeptide comprising 2A site
LNFDLLKLAGDVESNPGP

SEQ ID NO: 70 - protein, 40 aa, Artificial Sequence
  Self-cleaving polypeptide comprising 2A site
LLAIHPTEARHKQKIVAPVKQTLNFDLLKLAGDVESNPGP

SEQ ID NO: 71 - protein, 33 aa, Artificial Sequence
  Self-cleaving polypeptide comprising 2A site
EARHKQKIVAPVKQTLNFDLLKLAGDVESNPGP

SEQ ID NO: 72 - DNA, 10 bp, Artificial Sequence
  Consensus Kozak sequence
gccrccatgg

SEQ ID NO: 73 - DNA, 42 bp, Homo sapiens
tatgaaaactgagtttcaagatatcaaggacttggccttaga

SEQ ID NO: 74 - DNA, 22 bp, Homo sapiens
ccgcataggatttggccttagg

SEQ ID NO: 75 - DNA, 22 bp, Homo sapiens
atgacagggatatggccttagg

SEQ ID NO: 76 - DNA, 22 bp, Homo sapiens
ctgcacaggatttggcctcaga

SEQ ID NO: 77 - DNA, 22 bp, Homo sapiens
cagcataggatatagccttacc

SEQ ID NO: 78 - DNA, 22 bp, Homo sapiens
aggaacaggatttggccttagc

SEQ ID NO: 79 - DNA, 22 bp, Homo sapiens
gagataaggatatagccttaga

SEQ ID NO: 80 - DNA, 22 bp, Homo sapiens
ctgctgtggatttggccttata

SEQ ID NO: 81 - DNA, 22 bp, Homo sapiens
atgaaagggatttggccttaaa

SEQ ID NO: 82 - DNA, 22 bp, Homo sapiens
gggcacaggacttggccttatt

SEQ ID NO: 83 - DNA, 22 bp, Homo sapiens
atgccaaggatatgacctcatc

(19) United States
(12) Patent Application Publication
(10) Pub. No.: US 2021/0230565 A1
(43) Pub. Date: Jul. 29, 2021

(54) BRUTON'S TYROSINE KINASE HOMING ENDONUCLEASE VARIANTS, COMPOSITIONS, AND METHODS OF USE

(71) Applicants: Seattle Children's Hospital d/b/a Seattle Children's Research Institute, Seattle, WA (US); bluebird bio, Inc., Cambridge, MA (US)

(72) Inventors: David J. RAWLINGS, Seattle, WA (US); Yupeng WANG, Kenmore, WA (US); Iram F. KHAN, Issaquah, WA (US); Joel GAY, Port Angeles, WA (US)

(21) Appl. No.: 17/050,774

(22) PCT Filed: Apr. 26, 2019
(86) PCT No.: PCT/US2019/029414

§ 371 Date: Oct. 26, 2020

Related U.S. Application Data
(60) Provisional application No. 62/671,948, filed on May 15, 2018.
(60) Provisional application No. 62/663,982, filed on Apr. 27, 2018.

(51) Int. Cl. C12N 9/12; C12N 15/90; C12N 15/86; A61K 35/28

(52) U.S. Cl. CPC C12N 9/12; C12Y 207/10002; A61K 48/00; C12N 15/86; A61K 35/28; C12N 15/907

(57) ABSTRACT

The present disclosure provides improved genome editing compositions and methods for editing a human BTK gene. The disclosure further provides genome edited cells for the prevention, treatment, or amelioration of at least one symptom of X-linked agammaglobulinemia (XLA).

### CROSS REFERENCE TO RELATED APPLICATIONS

This application claims the benefit under 35 U.S.C. § 119(e) of U.S. Provisional Application No. 62/671,948, filed May 15, 2018, and U.S. Provisional Application No. 62/663,982, filed Apr. 27, 2018, each of which is incorporated by reference herein in its entirety.

### STATEMENT REGARDING SEQUENCE LISTING

The Sequence Listing associated with this application is provided in text format in lieu of a paper copy, and is hereby incorporated by reference into the specification. The name of the text file containing the Sequence Listing is BLBD_098_02WO_ST25.txt. The text file is 156 KB, was created on Apr. 26, 2019, and is being submitted electronically via EFS-Web, concurrent with the filing of the specification.

### BACKGROUND

### Technical Field

The present disclosure relates to improved genome editing compositions. More particularly, the disclosure relates to reprogrammed nucleases, compositions, and methods of using the same for editing the Bruton's tyrosine kinase (BTK) gene.

### Description of the Related Art

X-linked agammaglobulinemia is a rare immunodeficiency caused by mutations in the Bruton's tyrosine kinase (BTK) gene. More than 600 different mutations in the BTK gene have been linked to X-linked agammaglobulinemia. Most of these mutations result in the absence of the BTK protein. Other mutations change a single protein building block (amino acid), which can lead to abnormal BTK protein production that is quickly broken down in the cell. BTK is required for the normal B maturation and activation, for BCR-mediated signaling, am some signaling pathways in myeloid cells. Subjects lacking functional BTK have predominantly immature B cells, minimal antibody production, and are prone to recurrent and life-threatening infections.

Existing treatments include life-long intravenous immunoglobulin therapy, which lessens the severity of these infections, and judicious use of antibiotic therapy. Hematopoietic cell transplantation (HCT) is the only available approach with the potential of providing a cure for XLA. However, most XLA patients are not treated with this approach due to the difficult of finding HLA-matched donors and potential toxicities associated with GvHD. Despite significant improvements in transplant survival, the risk of treatment-related mortality has been a barrier to allo-HCT for XLA. Integrating self-inactivating lentiviral vectors (LV) encoding BTK cDNA under the control of the native proximal BTK gene promoter have been developed and evaluated in mouse model of human XLA. However, there are singificant risks of insertional mutagenesis and gene expression disregulation associated with retroviral and LV-based gene therapies.

### BRIEF SUMMARY

The present disclosure generally relates, in part, to compositions comprising homing endonuclease variants and megaTALs that cleave a target site in the human BTK gene and methods of using the same.

In various embodiments, a polypeptide comprises a homing endonuclease (HE) variant that cleaves a target site in the human Bruton's tyrosine kinas (BTK) gene.

In certain embodiments, the HE variant is an LAGLIDADG homing endonuclease (LHE) variant.

In particular embodiments, the polypeptide comprises a biologically active fragment of the HE variant.

In some embodiments, the biologically active fragment lacks the 1, 2, 3, 4, 5, 6, 7, or 8 N-terminal amino acids compared to a corresponding wild type HE.

In particular embodiments, the biologically active fragment lacks the 4 N-terminal amino acids compared to a corresponding wild type HE.

In various embodiments, the biologically active fragment lacks the 8 N-terminal amino acids compared to a corresponding wild type HE.

In further embodiments, the biologically active fragment lacks the 1, 2, 3, 4, or 5 C-terminal amino acids compared to a corresponding wild type HE.

In particular embodiments, the biologically active fragment lacks the C-terminal amino acid compared to a corresponding wild type HE.

In certain embodiments, the biologically active fragment lacks the 2 C-terminal amino acids compared to a corresponding wild type HE.

In various embodiments, the HE variant is a variant of an LHE selected from the group consisting of: I-AabMI, I-AaeMI, I-AniI, I-ApaMI, I-CapIII, I-CapIV, I-CkaMI, I-CpaMI, I-CpaMII, I-CpaMIII, I-CpaMIV, I-CpaMV, I-CpaV, I-CraMI, I-EjeMI, I-GpeMI, I-GpiI, I-GzeMI, I-GzeMII, I-GzeMIII, I-HjeMI, I-LtrII, I-LtrI, I-LtrWI, I-MpeMI, I-MveMI, I-NcrII, I-Ncrl, I-NcrMI, I-OheMI, I-OnuI, I-OsoMI, I-OsoMII, I-OsoMIII, I-OsoMIV, I-PanMI, I-PanMII, I-PanMIII, I-PnoMI, I-SceI, I-ScuMI, I-SmaMI, I-SscMI, and I-Vdi141I.

In particular embodiments, the HE variant is a variant of an LHE selected from the group consisting of: I-CpaMI, I-HjeMI, I-OnuI, I-PanMI, and I-SmaMI.

In various embodiments, the HE variant is an I-OnuI LHE variant.

In some embodiments, the HE variant comprises one or more amino acid substitutions at amino acid positions selected from the group consisting of: 24, 26, 28, 30, 32, 34, 35, 36, 37, 38, 40, 42, 44, 46, 48, 68, 70, 72, 75, 76, 78, 80, 82, 180, 182, 184, 186, 188, 189, 190, 191, 192, 193, 195, 197, 199, 201, 203, 223, 225, 227, 229, 231, 232, 234, 236, 238, and 240 of an I-OnuI LHE amino acid sequence as set forth in SEQ ID NOs: 1-5, or a biologically active fragment thereof.

In further embodiments, the HE variant comprises at least 5, at least 15, preferably at least 25, more preferably at least 35, or even more preferably at least 40 or more amino acid substitutions at amino acid positions selected from the group consisting of: 24, 26, 28, 30, 32, 34, 35, 36, 37, 38, 40, 42, 44, 46, 48, 68, 70, 72, 75, 76, 78, 80, 82, 180, 182, 184, 186, 188, 189, 190, 191, 192, 193, 195, 197, 199, 201, 203, 223, 225, 227, 229, 231, 232, 234, 236, 238, and 240 of an I-OnuI LHE amino acid sequence as set forth in SEQ ID NOs: 1-5, or a biologically active fragment thereof.

In particular embodiments, the HE variant comprises one or more amino acid substitutions at amino acid positions selected from the group consisting of: 24, 26, 28, 30, 32, 34, 35, 36, 37, 38, 40, 42, 44, 46, 48, 61, 68, 70, 72, 75, 76, 78, 80, 82, 85, 116, 135, 138, 143, 147, 159, 164, 168, 178, 180, 182, 184, 186, 188, 189, 190, 191, 192, 193, 195, 197, 199, 201, 203, 210, 223, 225, 227, 229, 231, 232, 234, 236, 238, 240, and 246 of an I-OnuI LHE amino acid sequence as set forth in SEQ ID NOs: 1-5, or a biologically active fragment thereof.

In certain embodiments, the HE variant comprises at least 5, at least 15, preferably at least 25, more preferably at least 35, or even more preferably at least 40 or more amino acid substitutions at amino acid positions selected from the group consisting of: 24, 26, 28, 30, 32, 34, 35, 36, 37, 38, 40, 42, 44, 46, 48, 61, 68, 70, 72, 75, 76, 78, 80, 82, 85, 116, 135, 138, 143, 147, 159, 164, 168, 178, 180, 182, 184, 186, 188, 189, 190, 191, 192, 193, 195, 197, 199, 201, 203, 210, 223, 225, 227, 229, 231, 232, 234, 236, 238, 240, and 246 of an I-OnuI LHE amino acid sequence as set forth in SEQ ID NOs: 1-17, or a biologically active fragment thereof.

In various embodiments, the HE variant comprises at least 5, at least 15, preferably at least 25, more preferably at least 35, or even more preferably at least 40 or more amino acid substitutions at amino acid positions selected from the group consisting of: 24, 26, 28, 30, 32, 34, 35, 36, 37, 38, 40, 42, 44, 46, 48, 61, 68, 70, 72, 75, 76, 78, 80, 82, 85, 116, 135, 138, 143, 147, 159, 164, 168, 178, 180, 182, 184, 186, 188, 189, 190, 191, 192, 193, 195, 197, 199, 201, 203, 210, 223, 225, 227, 229, 231, 232, 234, 236, 238, 240, and 246 of an I-OnuI LHE amino acid sequence as set forth in SEQ ID NOs: 1-5, or a biologically active fragment thereof.

In particular embodiments, the HE variant comprises at least 5, at least 15, preferably at least 25, more preferably at least 35, or even more preferably at least 40 or more of the following amino acid substitutions: S24W, L26M, L26S, R28V, R28D, N32S, K34T, S35V, S36K, S40R, E42L, G44S, Q46G, T48E, Q61R, V68K, A70S, A70R, N75H, N75R, A76Y, S78R, K80T, T82S, E85G, V116L, K135R, L138M, T143N, K147E, S159P, I161V, N164S, F168L, E178D, C180S, C180T, F182Y, I186V, S188G, S190N, K191T, L192T, G193R, Q195T, Q195Y, S201Q, S201G, N210Y, K225L, K229V, F232R, W234F, D236Q, V238R, and N246K, in reference to an I-OnuI LHE amino acid sequence as set forth in SEQ ID NOs: 1-5, or a biologically active fragment thereof.

In further embodiments, the HE variant comprises at least 5, at least 15, preferably at least 25, more preferably at least 35, or even more preferably at least 40 or more of the following amino acid substitutions: S24W, R28V, N32S, K34T, S35V, S36K, S40R, E42L, G44S, Q46G, T48E, V68K, A70S, N75H, A76Y, S78R, K80T, T82S, V116L, L138M, T143N, S159P, F168L, E178D, C180S, F182Y, I186V, S188G, S190N, K191T, L192T, G193R, Q195T, S201Q, K225L, K229V, F232R, W234F, D236Q, and V238R, in reference to an I-OnuI LHE amino acid sequence as set forth in SEQ ID NOs: 1-5, or a biologically active fragment thereof.

In various embodiments, the HE variant comprises at least 5, at least 15, preferably at least 25, more preferably at least 35, or even more preferably at least 40 or more of the following amino acid substitutions: S24W, R28V, N32S, K34T, S35V, S36K, S40R, E42L, G44S, Q46G, T48E, V68K, A70S, N75H, A76Y, S78R, K80T, T82S, V116L, K135R, L138M, T143N, S159P, F168L, E178D, C180S, F182Y, I186V, S188G, S190N, K191T, L192T, G193R, Q195T, S201Q, K225L, K229V, F232R, W234F, D236Q, V238R, and N246K, in reference to an I-OnuI LHE amino acid sequence as set forth in SEQ ID NOs: 1-5, or a biologically active fragment thereof.

In certain embodiments, the HE variant comprises at least 5, at least 15, preferably at least 25, more preferably at least 35, or even more preferably at least 40 or more of the following amino acid substitutions: S24W, R28D, N32S, K34T, S35V, S36K, S40R, E42L, G44S, Q46G, T48E, V68K, A70R, N75R, A76Y, K80T, T82S, V116L, L138M, T143N, S159P, N164S, F168L, E178D, C180S, F182Y, I186V, S188G, S190N, K191T, L192T, G193R, Q195T, S201Q, N210Y, K225L, K229V, F232R, W234F, D236Q, and V238R, in reference to an I-OnuI LHE amino acid sequence as set forth in SEQ ID NOs: 1-5, or a biologically active fragment thereof.

In various embodiments, the HE variant comprises at least 5, at least 15, preferably at least 25, more preferably at least 35, or even more preferably at least 40 or more of the following amino acid substitutions: S24W, R28V, N32S, K34T, S35V, S36K, S40R, E42L, G44S, Q46G, T48E, V68K, A70S, N75H, A76Y, S78R, K80T, T82S, L138M, T143N, S159P, F168L, E178D, C180T, F182Y, S188G, S190N, K191T, L192T, G193R, Q195Y, S201G, K225L, K229V, F232R, W234F, D236Q, and V238R, in reference to an I-OnuI LHE amino acid sequence as set forth in SEQ ID NOs: 1-5, or a biologically active fragment thereof.

In some embodiments, the HE variant comprises at least 5, at least 15, preferably at least 25, more preferably at least 35, or even more preferably at least 40 or more of the following amino acid substitutions: S24W, R28V, R28D, K34T, S35V, S36K, S40R, E42L, G44S, Q46G, T48E, V68K, A70S, N75H, A76Y, S78R, K80T, T82S, V116L, L138M, T143N, S159P, F168L, E178D, C180S, F182Y, S188G, S190N, K191T, L192T, G193R, Q195T, S201Q, K225L, K229V, F232R, W234F, D236Q, and V238R, in reference to an I-OnuI LHE amino acid sequence as set forth in SEQ ID NOs: 1-5, or a biologically active fragment thereof.

In further embodiments, the HE variant comprises at least 5, at least 15, preferably at least 25, more preferably at least 35, or even more preferably at least 40 or more of the following amino acid substitutions: S24W, R28D, N32S, K34T, S35V, S36K, S40R, E42L, G44S, Q46G, T48E, V68K, A70S, N75H, A76Y, S78R, K80T, T82S, V116L, L138M, T143N, S159P, F168L, E178D, C180T, F182Y, S188G, S190N, K191T, L192T, G193R, Q195Y, S201G, K225L, K229V, F232R, W234F, D236Q, and V238R, in reference to an I-OnuI LHE amino acid sequence as set forth in SEQ ID NOs: 1-5, or a biologically active fragment thereof.

In particular embodiments, the HE variant comprises at least 5, at least 15, preferably at least 25, more preferably at least 35, or even more preferably at least 40 or more of the following amino acid substitutions: S24W, L26M, R28D, N32S, K34T, S35V, S36K, S40R, E42L, G44S, Q46G, T48E, V68K, A70R, N75R, A76Y, K80T, T82S, V116L, L138M, T143N, K147E, S159P, F168L, E178D, C180T, F182Y, S188G, S190N, K191T, L192T, G193R, Q195Y, S201G, K225L, K229V, F232R, W234F, D236Q, and V238R, in reference to an I-OnuI LHE amino acid sequence as set forth in SEQ ID NOs: 1-5, or a biologically active fragment thereof.

In some embodiments, the HE variant comprises at least 5, at least 15, preferably at least 25, more preferably at least 35, or even more preferably at least 40 or more of the following amino acid substitutions: S24W, L26S, R28V, N32S, K34T, S35V, S36K, S40R, E42L, G44S, Q46G, T48E, V68K, A70S, N75R, S78R, K80T, E85G, V116L, L138M, T143N, S159P, F168L, E178D, C180T, F182Y, S188G, S190N, K191T, L192T, G193R, Q195Y, S201G, K225L, K229V, F232R, W234F, D236Q, and V238R, in reference to an I-OnuI LHE amino acid sequence as set forth in SEQ ID NOs: 1-5, or a biologically active fragment thereof.

In various embodiments, the HE variant comprises at least 5, at least 15, preferably at least 25, more preferably at least 35, or even more preferably at least 40 or more of the following amino acid substitutions: S24W, L26S, R28V, N32S, K34T, S35V, S36K, S40R, E42L, G44S, Q46G, T48E, V68K, A70S, N75R, S78R, K80T, E85G, V116L, L138M, T143N, S159P, F168L, E178D, C180T, F182Y, S188G, S190N, K191T, L192T, G193R, Q195Y, S201G, K225L, K229V, F232R, W234F, D236Q, and V238R, in reference to an I-OnuI LHE amino acid sequence as set forth in SEQ ID NOs: 1-5, or a biologically active fragment thereof.

In various embodiments, the HE variant comprises at least 5, at least 15, preferably at least 25, more preferably at least 35, or even more preferably at least 40 or more of the following amino acid substitutions: S24W, R28V, N32S, K34T, S35V, S36K, S40R, E42L, G44S, Q46G, T48E, V68K, A70S, N75H, A76Y, S78R K80T, T82S, V116L, L138M, T143N, S159P, F168L, E178D, C180T, F182Y, S188G, S190N, K191T, L192T, G193R, Q195Y, S201G, K225L, K229V, F232R, W234F, D236Q, and V238R, in reference to an I-OnuI LHE amino acid sequence as set forth in SEQ ID NOs: 1-5, or a biologically active fragment thereof.

In particular embodiments, the HE variant comprises at least 5, at least 15, preferably at least 25, more preferably at least 35, or even more preferably at least 40 or more of the following amino acid substitutions: S24W, L26S, R28V, N32S, K34T, S35V, S36K, S40R, E42L, G44S, Q46G, T48E, Q61R, V68K, A70S, N75R, S78R, K80T, V116L, L138M, T143N, S159P, F168L, E178D, C180S, F182Y, S188G, S190N, K191T, L192T, G193R, Q195Y, S201G, K225L, K229V, F232R, W234F, D236Q, and V238R, in reference to an I-OnuI LHE amino acid sequence as set forth in SEQ ID NOs: 1-5, or a biologically active fragment thereof.

In certain embodiments, the HE variant comprises at least 5, at least 15, preferably at least 25, more preferably at least 35, or even more preferably at least 40 or more of the following amino acid substitutions: S24W, R28D, N32S, K34T, S35V, S36K, S40R, E42L, G44S, Q46G, T48E, V68K, A70S, N75H, A76Y, S78R, K80T, T82S, V116L, L138M, T143N, K147E, S159P, I161V, F168L, E178D, C180T, F182Y, S188G, S190N, K191T, L192T, G193R, Q195Y, S201G, K225L, K229V, F232R, W234F, D236Q, and V238R, in reference to an I-OnuI LHE amino acid sequence as set forth in SEQ ID NOs: 1-5, or a biologically active fragment thereof.

In further embodiments, the HE variant comprises an amino acid sequence that is at least 80%, preferably at least 85%, more preferably at least 90%, or even more preferably at least 95% identical to the amino acid sequence set forth in any one of SEQ ID NOs: 6-17, or a biologically active fragment thereof.

In particular embodiments, the HE variant comprises the amino acid sequence set forth in SEQ ID NO: 6, or a biologically active fragment thereof.

In further embodiments, the HE variant comprises the amino acid sequence set forth in SEQ ID NO: 7, or a biologically active fragment thereof.

In various embodiments, the HE variant comprises the amino acid sequence set forth in SEQ ID NO: 8, or a biologically active fragment thereof.

In particular embodiments, the HE variant comprises the amino acid sequence set forth in SEQ ID NO: 9, or a biologically active fragment thereof.

In some embodiments, the HE variant comprises the amino acid sequence set forth in SEQ ID NO: 10, or a biologically active fragment thereof.

In particular embodiments, the HE variant comprises the amino acid sequence set forth in SEQ ID NO: 11, or a biologically active fragment thereof.

In various embodiments, the HE variant comprises the amino acid sequence set forth in SEQ ID NO: 12, or a biologically active fragment thereof.

In some embodiments, the HE variant comprises the amino acid sequence set forth in SEQ ID NO: 13, or a biologically active fragment thereof.

In various embodiments, the HE variant comprises the amino acid sequence set forth in SEQ ID NO: 14, or a biologically active fragment thereof.

In further embodiments, the HE variant comprises the amino acid sequence set forth in SEQ ID NO: 15, or a biologically active fragment thereof.

In various embodiments, the HE variant comprises the amino acid sequence set forth in SEQ ID NO: 16, or a biologically active fragment thereof.

In certain embodiments, the HE variant comprises the amino acid sequence set forth in SEQ ID NO: 17, or a biologically active fragment thereof.

In particular embodiments, the HE variant binds a polynucleotide sequence in the BTK gene.

In some embodiments, the HE variant binds the polynucleotide sequence set forth in SEQ ID NO: 24.

In further embodiments, a polypeptide contemplated herein further comprises a DNA binding domain.

In certain embodiments, the DNA binding domain is selected from the group consisting of: a TALE DNA binding domain and a zinc finger DNA binding domain.

In particular embodiments, the TALE DNA binding domain comprises about 9.5 TALE repeat units to about 15.5 TALE repeat units.

In further embodiments, the TALE DNA binding domain binds a polynucleotide sequence in the BTK gene.

In some embodiments, the TALE DNA binding domain binds the polynucleotide sequence set forth in SEQ ID NO: 25.

In various embodiments, the zinc finger DNA binding domain comprises 2, 3, 4, 5, 6, 7, or 8 zinc finger motifs.

In particular embodiments, a polypeptide contemplated herein further comprises a peptide linker and an end-processing enzyme or biologically active fragment thereof.

In further embodiments, a polypeptide contemplated herein further comprises a viral self-cleaving 2A peptide and an end-processing enzyme or biologically active fragment thereof.

In some embodiments, the end-processing enzyme or biologically active fragment thereof has 5′-3′ exonuclease, 5′-3′ alkaline exonuclease, 3′-5′ exonuclease, 5′ flap endonuclease, helicase, template-dependent DNA polymerase or template-independent DNA polymerase activity.

In further embodiments, the end-processing enzyme comprises Trex2 or a biologically active fragment thereof.

In various embodiments, the polypeptide cleaves the human BTK gene at the polynucleotide sequence set forth in SEQ ID NO: 24 or SEQ ID NO: 26.

In some embodiments, a polynucleotide encodes a polypeptide contemplated herein.

In further embodiments, an mRNA encodes a polypeptide contemplated herein.

In particular embodiments, a cDNA encodes a polypeptide contemplated herein.

In various embodiments, a vector comprises a polynucleotide encoding a polypeptide contemplated herein.

In some embodiments, a cell comprises a polypeptide contemplated herein.

In certain embodiments, a cell comprises a polynucleotide encoding a polypeptide contemplated herein.

In certain embodiments, a cell comprises a vector contemplated herein.

In various embodiments, a cell comprises one or more genome modifications introduced by a polypeptide contemplated herein.

In particular embodiments, the cell is a hematopoietic cell.

In particular embodiments, the cell is a hematopoietic stem or progenitor cell.

In particular embodiments, the cell is a CD34+ cell.

In further embodiments, the cell is a CD133+ cell.

In some embodiments, a composition comprises a cell comprising one or more genome modifications introduced by a polypeptide contemplated herein.

In various embodiments, a composition comprises a cell comprising one or more genome modifications contemplated herein and a physiologically acceptable carrier.

In certain embodiments, a method of editing a BTK gene in a cell comprises: introducing a polypeptide, a polynucleotide encoding a polypeptide, or a vector contemplated herein; and a donor repair template into the cell, wherein expression of the polypeptide creates a double strand break at a target site in a BTK gene and the donor repair template is incorporated into the BTK gene by homology directed repair (HDR) at the site of the double-strand break (DSB).

In some embodiments, the BTK gene comprises one or more amino acid mutations or deletions that result in X-linked agammaglobulinemia (XLA).

In particular embodiments, the cell is a hematopoietic cell.

In further embodiments, the cell is a hematopoietic stem or progenitor cell.

In particular embodiments, the cell is a CD34+ cell.

In various embodiments, the cell is a CD133+ cell.

In certain embodiments, the polynucleotide encoding the polypeptide is an mRNA.

In various embodiments, a polynucleotide encoding a 5′-3′ exonuclease is introduced into the cell.

In further embodiments, a polynucleotide encoding Trex2 or a biologically active fragment thereof is introduced into the cell.

In some embodiments, the donor repair template comprises a 5′ homology arm homologous to a BTK gene sequence 5′ of the DSB, a donor polynucleotide, and a 3′ homology arm homologous to a BTK gene sequence 3′ of the DSB.

In various embodiments, the donor polynucleotide is designed to repair one or more amino acid mutations or deletions in the BTK gene.

In particular embodiments, the donor polynucleotide comprises a cDNA encoding a BTK polypeptide.

In further embodiments, the donor polynucleotide comprises an expression cassette comprising a promoter operable linked to a cDNA encoding a BTK polypeptide.

In particular embodiments, the lengths of the 5′ and 3′ homology arms are independently selected from about 100 bp to about 2500 bp.

In various embodiments, the lengths of the 5′ and 3′ homology arms are independently selected from about 600 bp to about 1500 bp.

In some embodiments, the 5′homology arm is about 1500 bp and the 3′ homology arm is about 1000 bp.

In certain embodiments, the 5′homology arm is about 600 bp and the 3′ homology arm is about 600 bp.

In further embodiments, a viral vector is used to introduce the donor repair template into the cell.

In certain embodiments, the viral vector is a recombinant adeno-associated viral vector (rAAV) or a retrovirus.

In various embodiments, the rAAV has one or more ITRs from AAV2.

In further embodiments, the rAAV has a serotype selected from the group consisting of: AAV1, AAV2, AAV3, AAV4, AAV5, AAV6, AAV7, AAV8, AAV9, and AAV10.

In particular embodiments, the rAAV has an AAV2 or AAV6 serotype.

In some embodiments, the retrovirus is a lentivirus.

In certain embodiments, the lentivirus is an integrase deficient lentivirus (IDLY).

In particular embodiments, a method of treating, preventing, or ameliorating at least one symptom of X-linked agammaglobulinemia (XLA), or condition associated therewith, comprises harvesting a population of cells from the subject; editing the population of cells according to a method of editing a BTK gene contemplated herein, and administering the edited population of cells to the subject.

### BRIEF DESCRIPTION OF SEVERAL VIEWS OF THE DRAWINGS

FIG. 1 shows a cartoon of the BTK megaTAL recognition site in intron 2 (SEQ ID NO: 73) of human Bruton's tyrosine kinase (BTK) gene. The recognition site 30 base pairs (bp) downstream of exon 2 and 175 bp downstream of translation start codon.

FIG. 2 shows cleavage activity of I-OnuI BTK variants in a yeast surface display assay under pH8 and pH7.

FIG. 3 shows the cleavage activity of I-OnuI BTK variants linked to BFP reformatted as TREX2 fusions or megaTALs. FIGS. 3A and 3B show the that the reformatted I-OnuI BTK variants have compared expression levels (% BFP expression). FIGS. 3C and 3D show the cleavage activity of the reformatted I-OnuI BTK variants as % mCherry expression.

FIG. 4 shows the cleavage efficiency of three I-OnuI BTK megaTALs MTBTK_L4_V25, MTBTK_EL4_V34, and MTBTK_EL4_V42 in a T7 endonuclease assay. FIG. 4A shows the cleavage efficiency of MTBTK_L4_V25, MTBTK_EL4_V34, and MTBTK_EL4_V42 in Jurkat cells. FIG. 4B shows the cleavage efficiency of MTBTK_L4_V25, MTBTK_EL4_V34, and MTBTK_EL4_V42 in human primary CD4+ T cells. FIG. 4C shows the cleavage efficiency of MTBTK_L4_V25, MTBTK_EL4_V34, and MTBTK_EL4_V42 in human CD34+ cells. FIG. 4D shows the cleavage efficiencies of FIGS. 4A-4C.

FIG. 5 shows homology-directed-repair (HDR) induced in human primary CD4+ T cells by transfected with BTK megaTALs MTBTK_L4_V25, MTBTK_EL4_V34, and MTBTK_EL4_V42 and an AAV GFP-expressing donor repair template. FIG. 5A shows a cartoon of the experimental set up. FIG. 5B shows a cartoon of the HDR strategy. FIG. 5C shows the viability of CD4+ T cells at day 2 and day 15 after transfection. FIG. 5D shows GFP expression in CD4+ T cells at day 2 and day 15 after transfection. Data presented is representative of two independent experiments.

FIG. 6 shows homology-directed-repair (HDR) induced in human primary CD4+ T cells by transfected with MTBTK_EL4_V34 megaTAL and an AAV GFP-expressing donor repair template. FIG. 6A shows a cartoon of the experimental set up. FIG. 6B shows a cartoon of the HDR strategy. FIG. 6C shows the viability of CD4+ T cells at day 2 and day 15 after transfection. FIG. 6D shows GFP expression in CD4+ T cells at day 2 and day 15 after transfection. Data presented is the average of two independent experiments.

FIG. 7 shows homology-directed-repair (HDR) induced in human primary CD34⁺ T cells by transfected with BTK megaTAL MTBTK_EL4_V34 and an AAV GFP-expressing donor repair template. FIG. 7A shows a schematic illustration of an experimental approach of editing the BTK gene in CD34⁺ cells. FIG. 7B shows the viability of CD34 cells at day 1 and day 5 after mRNA electroporation and BTK mTAL-specific AAV transduction. FIG. 7C shows GFP expression at day 1 and day 5 after mRNA electroporation and BTK mTAL-specific AAV transduction.

FIG. 8 shows the viability of CD34⁺ cell one day post gene editing using BTK mTAL with and without rAAV6 donor. FIG. 8A shows a schematic illustration of an experimental approach of editing the BTK gene in CD34⁺ cells. FIG. 8B shows a schematic of the HDR strategy at the human BTK locus. FIG. 8C shows the % viability of mock treated CD34⁺ cells and CD34⁺ cells transfected with 1 μg of BTK megaTAL MTBTK_EL4_V34 mRNA followed by the addition of rAAV6 donor template (3% total culture volume), CD34⁺ cells transfected with 1 μg of mTAL mRNA, and CD34⁺ cells treated with 3% culture volumes of rAAV6 donor template. Different donors are represented by differently hatched circles (n=4 independent donors).

FIG. 9 shows homology-directed-repair (HDR) induced in human primary CD34⁺ T cells by transfected with BTK megaTAL MTBTK_EL4_V34 and an AAV GFP-expressing donor repair template. FIG. 9A shows representative flow cytometry plots depicting viability and GFP expression on days 1 and 5 post editing. FIG. 9B shows the % HDR measured by FACS compared to % HDR determined by droplet digital PCR (ddPCR) 5 days post editing. FIG. 9C shows the ratio of HDR to NHEJ.

FIG. 10 shows that colony formation is substantially similar in BTK mTAL editing in CD34⁺ cells compared to mock edited cells. CFU-E: Colony forming unit erythroid, M: Macrophage, GM: Granulocyte, macrophage, G: Granulocyte, GEMM: Granulocyte, erythroid, macrophage, megakaryocyte, BFU-E: Burst forming unit erythroid.

FIG. 11 shows an HDR editing strategy using a donor repair template encoding a codon optimized BTK cDNA, truncated WPRE and SV40 polyA site. FIG. 11A shows a cartoon of the editing strategy. FIG. 11B shows HDR rates in mock-treated cells and cells treated with AAV donor repair template alone or with AAV donor repair template and BTK megaTAL mRNA.

FIG. 12 shows BTK megaTAL MTBTK_EL4_V34 specificity. FIG. 12A shows a specificity map. FIG. 12B shows the top ten off-target site sequences (SEQ ID NOs: 74-83) based on Guide-Seq. FIG. 12C shows a gel analysis of PCR amplicons of putative off-target sites identified by GUIDE-Seq in T cells edited with BTK megaTAL MTBTK_EL4_V34. FIG. 12D is a table of the NHEJ rates at the putative top ten off-target sites.

### BRIEF DESCRIPTION OF THE SEQUENCE IDENTIFIERS

SEQ ID NO: 1 is an amino acid sequence of a wild type I-OnuI LAGLIDADG homing endonuclease (LHE).

SEQ ID NO: 2 is an amino acid sequence of a wild type I-OnuI LHE.

SEQ ID NO: 3 is an amino acid sequence of a biologically active fragment of a wild-type I-OnuI LHE.

SEQ ID NO: 4 is an amino acid sequence of a biologically active fragment of a wild-type I-OnuI LHE.

SEQ ID NO: 5 is an amino acid sequence of a biologically active fragment of a wild-type I-OnuI LHE.

SEQ ID NOs: 6-17 are amino acid sequences of I-OnuI LHE variants reprogrammed to bind and cleave a target site in the human BTK gene.

SEQ ID NOs: 18-20 are amino acid sequences of megaTALs that bind and cleave a target site in the human BTK gene.

SEQ ID NOs: 21-23 are amino acid sequences of megaTAL-TREX2 fusions that bind and cleave a target site in the human BTK gene.

SEQ ID NO: 24 is an I-OnuI LHE variant target site in intron 2 of the human BTK gene.

SEQ ID NO: 25 is a TALE DNA binding domain target site in intron 2 of the human BTK gene.

SEQ ID NO: 26 is a megaTAL target site in intron 2 of the human BTK gene.

SEQ ID NOs: 27-29 are mRNA sequences encoding megaTALs that cleave a target site in intron 2 of the human BTK gene.

SEQ ID NO: 30 is an mRNA sequence that encodes a TREX2 protein.

SEQ ID NO: 31 is an amino acid sequence of a TREX2 protein.

SEQ ID NO: 32 is an amino acid sequence of a human BTK polypeptide.

SEQ ID NO: 33 is a representative AAV donor repair template for the BTK locus.

SEQ ID NO: 34 is a representative AAV donor repair template for the BTK locus.

SEQ ID NO: 35 is a representative AAV donor repair template for the BTK locus.

SEQ ID NOs: 36-46 set forth the amino acid sequences of various linkers.

SEQ ID NOs: 47-71 set forth the amino acid sequences of protease cleavage sites and self-cleaving polypeptide cleavage sites. In the foregoing sequences, X, if present, refers to any amino acid or the absence of an amino acid.

### DETAILED DESCRIPTION

### A. Overview

The present disclosure generally relates to, in part, improved genome editing compositions and methods of use thereof. Without wishing to be bound by any particular theory, the genome editing compositions contemplated herein are used to increase the amount of Bruton's tyrosine kinase (BTK) in a cell to treat, prevent, or ameliorate symptoms associated with X-linked agammaglobulinemia (XLA). Thus, the compositions contemplated herein offer a potentially curative solution to subjects that have XLA. Without wishing to be bound to any particular theory, it is contemplated that a gene editing approach that introduces a polynucleotide encoding a functional BTK protein into a BTK gene that has one or more mutations and/or deletions that leads to XLA, will rescue the immunologic and functional deficits caused by XLA and to provide a potentially curative therapy.

In various embodiments, genome editing strategies, compositions, genetically modified cells, and methods of use thereof to increase or restore BTK function are contemplated. Without wishing to be bound by any particular theory, it is contemplated that genome editing of the BTK gene to introduce a polynucleotide encoding a functional copy of the BTK protein. In one embodiment, editing the BTK gene comprises introducing a polynucleotide encoding a functional copy of the BTK protein in such a way that it is under control of the endogenous promoter and enhancer in hematopoietic stem cells (HSC). Restoration of functional BTK in immune cells will effectively treat, prevent, and/or ameliorate one or more symptoms associated with subjects that have XLA.

Genome editing methods contemplated in various embodiments comprise nuclease variants, designed to bind and cleave a transcription factor binding site in the BTK gene. The nuclease variants contemplated in particular embodiments, can be used to introduce a double-strand break in a target polynucleotide sequence, and in the presence of a polynucleotide template, e.g., a donor repair template, result in homology directed repair (HDR), i.e., homologous recombination of the donor repair template into the BTK gene. Nuclease variants contemplated in certain embodiments, can also be designed as nickases, which generate single-stranded DNA breaks that can be repaired using the cell's base-excision-repair (BER) machinery or homologous recombination in the presence of a donor repair template. Homologous recombination requires homologous DNA as a template for repairing the double-stranded DNA break and can be leveraged to create a limitless variety of modifications specified by the introduction of donor DNA comprising an expression cassette or polynucleotide encoding a therapeutic gene, e.g., BTK, at the target site, flanked on either side by sequences bearing homology to regions flanking the target site.

In one preferred embodiment, the genome editing compositions contemplated herein comprise homing endonuclease variants or megaTALs that target the human BTK gene.

In various embodiments, wherein a DNA break is generated in the second intron of the BTK gene and a donor repair template, i.e., a donor repair template, comprising a polynucleotide encoding a functional BTK polypeptide is provided, the DSB is repaired with the sequence of the template by homologous recombination at the DNA break-site. In preferred embodiments, the repair template comprises a polynucleotide sequence that encodes a functional BTK polypeptide designed to be inserted at a site where the expression of the polynucleotide and BTK polypeptide is under the control of the endogenous BTK promoter and/or enhancers.

In one preferred embodiment, the genome editing compositions contemplated herein comprise nuclease variants and one or more end-processing enzymes to increase HDR efficiency.

In one preferred embodiment, the genome editing compositions contemplated herein comprise a homing endonuclease variant or megaTAL that targets a human BTK gene, a donor repair template encoding a functional BTK protein, and an end-processing enzyme, e.g., Trex2.

In various embodiments, genome edited cells are contemplated. The genome edited cells comprise a functional BTK polypeptide, rescue B cell development, and prevent XLA.

Accordingly, the methods and compositions contemplated herein represent a quantum improvement compared to existing gene editing strategies for the treatment of XLA.

Techniques for recombinant (i.e., engineered) DNA, peptide and oligonucleotide synthesis, immunoassays, tissue culture, transformation (e.g., electroporation, lipofection), enzymatic reactions, purification and related techniques and procedures may be generally performed as described in various general and more specific references in microbiology, molecular biology, biochemistry, molecular genetics, cell biology, virology and immunology as cited and discussed throughout the present specification. See, e.g., Sambrook et al., Molecular Cloning: A Laboratory Manual, 3d ed., Cold Spring Harbor Laboratory Press, Cold Spring Harbor, N.Y.; Current Protocols in Molecular Biology (John Wiley and Sons, updated July 2008); Short Protocols in Molecular Biology: A Compendium of Methods from Current Protocols in Molecular Biology, Greene Pub. Associates and Wiley-Interscience; Glover, DNA Cloning: A Practical Approach, vol. I & II (IRL Press, Oxford Univ. Press USA, 1985); Current Protocols in Immunology (Edited by: John E. Coligan, Ada M. Kruisbeek, David H. Margulies, Ethan M. Shevach, Warren Strober 2001 John Wiley & Sons, NY, NY); Real-Time PCR: Current Technology and Applications, Edited by Julie Logan, Kirstin Edwards and Nick Saunders, 2009, Caister Academic Press, Norfolk, UK; Anand, Techniques for the Analysis of Complex Genomes, (Academic Press, New York, 1992); Guthrie and Fink, Guide to Yeast Genetics and Molecular Biology (Academic Press, New York, 1991); Oligonucleotide Synthesis (N. Gait, Ed., 1984); Nucleic Acid The Hybridization (B. Hames & S. Higgins, Eds., 1985); Transcription and Translation (B. Hames & S. Higgins, Eds., 1984); Animal Cell Culture (R. Freshney, Ed., 1986); Perbal, A Practical Guide to Molecular Cloning (1984); Next-Generation Genome Sequencing (Janitz, 2008 Wiley-VCH); PCR Protocols (Methods in Molecular Biology) (Park, Ed., 3rd Edition, 2010 Humana Press); Immobilized Cells And Enzymes (IRL Press, 1986); the treatise, Methods In Enzymology (Academic Press, Inc., N.Y.); Gene Transfer Vectors For Mammalian Cells (J. H. Miller and M. P. Calos eds., 1987, Cold Spring Harbor Laboratory); Harlow and Lane, Antibodies, (Cold Spring Harbor Laboratory Press, Cold Spring Harbor, N.Y., 1998); Immunochemical Methods In Cell And Molecular Biology (Mayer and Walker, eds., Academic Press, London, 1987); Handbook Of Experimental Immunology, Volumes I-IV (D. M. Weir and CC Blackwell, eds., 1986); Roitt, Essential Immunology, 6th Edition, (Blackwell Scientific Publications, Oxford, 1988); Current Protocols in Immunology (Q. E. Coligan, A. M. Kruisbeek, D. H. Margulies, E. M. Shevach and W. Strober, eds., 1991); Annual Review of Immunology; as well as monographs in journals such as Advances in Immunology.

### B. Definitions

Prior to setting forth this disclosure in more detail, it may be helpful to an understanding thereof to provide definitions of certain terms to be used herein.

Unless defined otherwise, all technical and scientific terms used herein have the same meaning as commonly understood by those of ordinary skill in the art to which the invention belongs. Although any methods and materials similar or equivalent to those described herein can be used in the practice or testing of particular embodiments, preferred embodiments of compositions, methods and materials are described herein. For the purposes of the present disclosure, the following terms are defined below. Additional definitions are set forth throughout this disclosure.

The articles “a,” “an,” and “the” are used herein to refer to one or to more than one (i.e., to at least one, or to one or more) of the grammatical object of the article. By way of example, “an element” means one element or one or more elements.

The use of the alternative (e.g., “or”) should be understood to mean either one, both, or any combination thereof of the alternatives.

The term “and/or” should be understood to mean either one, or both of the alternatives.

As used herein, the term “about” or “approximately” refers to a quantity, level, value, number, frequency, percentage, dimension, size, amount, weight or length that varies by as much as 15%, 10%, 9%, 8%, 7%, 6%, 5%, 4%, 3%, 2% or 1% to a reference quantity, level, value, number, frequency, percentage, dimension, size, amount, weight or length. In one embodiment, the term “about” or “approximately” refers a range of quantity, level, value, number, frequency, percentage, dimension, size, amount, weight or length±15%, ±10%, ±9%, ±8%, ±7%, ±6%, ±5%, ±4%, ±3%, ±2%, or ±1% about a reference quantity, level, value, number, frequency, percentage, dimension, size, amount, weight or length.

In one embodiment, a range, e.g., 1 to 5, about 1 to 5, or about 1 to about 5, refers to each numerical value encompassed by the range. For example, in one non-limiting and merely illustrative embodiment, the range “1 to 5” is equivalent to the expression 1, 2, 3, 4, 5; or 1.0, 1.5, 2.0, 2.5, 3.0, 3.5, 4.0, 4.5, or 5.0; or 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, or 5.0.

As used herein, the term “substantially” refers to a quantity, level, value, number, frequency, percentage, dimension, size, amount, weight or length that is 80%, 85%, 90%, 91%, 92%, 93%, 94%, 95%, 96%, 97%, 98%, 99% or higher compared to a reference quantity, level, value, number, frequency, percentage, dimension, size, amount, weight or length. In one embodiment, “substantially the same” refers to a quantity, level, value, number, frequency, percentage, dimension, size, amount, weight or length that produces an effect, e.g., a physiological effect, that is approximately the same as a reference quantity, level, value, number, frequency, percentage, dimension, size, amount, weight or length.

Throughout this specification, unless the context requires otherwise, the words “comprise”, “comprises” and “comprising” will be understood to imply the inclusion of a stated step or element or group of steps or elements but not the exclusion of any other step or element or group of steps or elements. By “consisting of” is meant including, and limited to, whatever follows the phrase “consisting of” Thus, the phrase “consisting of” indicates that the listed elements are required or mandatory, and that no other elements may be present. By “consisting essentially of” is meant including any elements listed after the phrase, and limited to other elements that do not interfere with or contribute to the activity or action specified in the disclosure for the listed elements. Thus, the phrase “consisting essentially of” indicates that the listed elements are required or mandatory, but that no other elements are present that materially affect the activity or action of the listed elements.

Reference throughout this specification to “one embodiment,” “an embodiment,” “a particular embodiment,” “a related embodiment,” “a certain embodiment,” “an additional embodiment,” or “a further embodiment” or combinations thereof means that a particular feature, structure or characteristic described in connection with the embodiment is included in at least one embodiment. Thus, the appearances of the foregoing phrases in various places throughout this specification are not necessarily all referring to the same embodiment. Furthermore, the particular features, structures, or characteristics may be combined in any suitable manner in one or more embodiments. It is also understood that the positive recitation of a feature in one embodiment, serves as a basis for excluding the feature in a particular embodiment.

The term “ex vivo” refers generally to activities that take place outside an organism, such as experimentation or measurements done in or on living tissue in an artificial environment outside the organism, preferably with minimum alteration of the natural conditions. In particular embodiments, “ex vivo” procedures involve living cells or tissues taken from an organism and cultured or modulated in a laboratory apparatus, usually under sterile conditions, and typically for a few hours or up to about 24 hours, but including up to 48 or 72 hours, depending on the circumstances. In certain embodiments, such tissues or cells can be collected and frozen, and later thawed for ex vivo treatment. Tissue culture experiments or procedures lasting longer than a few days using living cells or tissue are typically considered to be “in vitro,” though in certain embodiments, this term can be used interchangeably with ex vivo.

The term “in vivo” refers generally to activities that take place inside an organism. In one embodiment, cellular genomes are engineered, edited, or modified in vivo.

By “enhance” or “promote” or “increase” or “expand” or “potentiate” refers generally to the ability of a nuclease variant, genome editing composition, or genome edited cell contemplated herein to produce, elicit, or cause a greater response (i.e., physiological response) compared to the response caused by either vehicle or control. A measurable response may include an increase in HDR, and/or BTK expression, among others apparent from the understanding in the art and the description herein. An “increased” or “enhanced” amount is typically a “statistically significant” amount, and may include an increase that is 1.1, 1.2, 1.5, 2, 3, 4, 5, 6, 7, 8, 9, 10, 15, 20, 30 or more times (e.g., 500, 1000 times) (including all integers and decimal points in between and above 1, e.g., 1.5, 1.6, 1.7, 1.8, etc.) the response produced by vehicle or control.

By “decrease” or “lower” or “lessen” or “reduce” or “abate” or “ablate” or “inhibit” or “dampen” refers generally to the ability of nuclease variant, genome editing composition, or genome edited cell contemplated herein to produce, elicit, or cause a lesser response (i.e., physiological response) compared to the response caused by either vehicle or control. A measurable response may include a decrease in one or more symptoms associated with XLA. A “decrease” or “reduced” amount is typically a “statistically significant” amount, and may include a decrease that is 1.1, 1.2, 1.5, 2, 3, 4, 5, 6, 7, 8, 9, 10, 15, 20, 30 or more times (e.g., 500, 1000 times) (including all integers and decimal points in between and above 1, e.g., 1.5, 1.6, 1.7, 1.8, etc.) the response (reference response) produced by vehicle, or control.

By “maintain,” or “preserve,” or “maintenance,” or “no change,” or “no substantial change,” or “no substantial decrease” refers generally to the ability of a nuclease variant, genome editing composition, or genome edited cell contemplated herein to produce, elicit, or cause a substantially similar or comparable physiological response (i.e., downstream effects) in as compared to the response caused by either vehicle or control. A comparable response is one that is not significantly different or measurable different from the reference response.

The terms “specific binding affinity” or “specifically binds” or “specifically bound” or “specific binding” or “specifically targets” as used herein, describe binding of one molecule to another, e.g., DNA binding domain of a polypeptide binding to DNA, at greater binding affinity than background binding. A binding domain “specifically binds” to a target site if it binds to or associates with a target site with an affinity or Kₐ (i.e., an equilibrium association constant of a particular binding interaction with units of 1/M) of, for example, greater than or equal to about 10⁵M⁻¹. In certain embodiments, a binding domain binds to a target site with a Kₐ greater than or equal to about 10⁶ M⁻¹, 10⁷ M⁻¹, 10⁸ M⁻¹, 10⁹M⁻¹, 10¹⁰ M⁻¹, 10¹¹M⁻¹, 10¹² M⁻¹, or 10¹³M⁻¹. “High affinity” binding domains refers to those binding domains with a Kₐ of at least 10⁷ M⁻¹, at least 10⁸M⁻¹, at least 10⁹ M⁻¹, at least 10¹⁰ M⁻¹, at least 10¹¹M⁻¹, at least 10¹² M⁻¹, at least 10¹³M⁻¹, or greater.

Alternatively, affinity may be defined as an equilibrium dissociation constant (K_{d}) of a particular binding interaction with units of M (e.g., 10⁻⁵M to 10⁻¹³ M, or less). Affinities of nuclease variants comprising one or more DNA binding domains for DNA target sites contemplated in particular embodiments can be readily determined using conventional techniques, e.g., yeast cell surface display, or by binding association, or displacement assays using labeled ligands.

In one embodiment, the affinity of specific binding is about 2 times greater than background binding, about 5 times greater than background binding, about 10 times greater than background binding, about 20 times greater than background binding, about 50 times greater than background binding, about 100 times greater than background binding, or about 1000 times greater than background binding or more.

The terms “selectively binds” or “selectively bound” or “selectively binding” or “selectively targets” and describe preferential binding of one molecule to a target molecule (on-target binding) in the presence of a plurality of off-target molecules. In particular embodiments, an HE or megaTAL selectively binds an on-target DNA binding site about 5, 10, 15, 20, 25, 50, 100, or 1000 times more frequently than the HE or megaTAL binds an off-target DNA target binding site.

“On-target” refers to a target site sequence.

“Off-target” refers to a sequence similar to but not identical to a target site sequence.

A “target site” or “target sequence” is a chromosomal or extrachromosomal nucleic acid sequence that defines a portion of a nucleic acid to which a binding molecule will bind and/or cleave, provided sufficient conditions for binding and/or cleavage exist. When referring to a polynucleotide sequence or SEQ ID NO. that references only one strand of a target site or target sequence, it would be understood that the target site or target sequence bound and/or cleaved by a nuclease variant is double-standard and comprises the reference sequence and its complement. In a preferred embodiment, the target site is a sequence in the human BTK gene.

“Recombination” refers to a process of exchange of genetic information between two polynucleotides, including but not limited to, donor capture by non-homologous end joining (NHEJ) and homologous recombination. For the purposes of this disclosure, “homologous recombination (HR)” refers to the specialized form of such exchange that takes place, for example, during repair of double-strand breaks in cells via homology-directed repair (HDR) mechanisms. This process requires nucleotide sequence homology, uses a “donor” molecule as a template to repair a “target” molecule (i.e., the one that experienced the double-strand break), and is variously known as “non-crossover gene conversion” or “short tract gene conversion,” because it leads to the transfer of genetic information from the donor to the target. Without wishing to be bound by any particular theory, such transfer can involve mismatch correction of heteroduplex DNA that forms between the broken target and the donor, and/or “synthesis-dependent strand annealing,” in which the donor is used to resynthesize genetic information that will become part of the target, and/or related processes. Such specialized HR often results in an alteration of the sequence of the target molecule such that part or all of the sequence of the donor polynucleotide is incorporated into the target polynucleotide.

“Cleavage” refers to the breakage of the covalent backbone of a DNA molecule. Cleavage can be initiated by a variety of methods including, but not limited to, enzymatic or chemical hydrolysis of a phosphodiester bond. Both single-stranded cleavage and double-stranded cleavage are possible. Double-stranded cleavage can occur as a result of two distinct single-stranded cleavage events. DNA cleavage can result in the production of either blunt ends or staggered ends. In certain embodiments, polypeptides and nuclease variants, e.g., homing endonuclease variants, megaTALs, etc. contemplated herein are used for targeted double-stranded DNA cleavage. Endonuclease cleavage recognition sites may be on either DNA strand.

An “exogenous” molecule is a molecule that is not normally present in a cell, but that is introduced into a cell by one or more genetic, biochemical or other methods. Exemplary exogenous molecules include, but are not limited to small organic molecules, protein, nucleic acid, carbohydrate, lipid, glycoprotein, lipoprotein, polysaccharide, any modified derivative of the above molecules, or any complex comprising one or more of the above molecules. Methods for the introduction of exogenous molecules into cells are known to those of skill in the art and include, but are not limited to, lipid-mediated transfer (i.e., liposomes, including neutral and cationic lipids), electroporation, direct injection, cell fusion, particle bombardment, biopolymer nanoparticle, calcium phosphate co-precipitation, DEAE-dextran-mediated transfer and viral vector-mediated transfer.

An “endogenous” molecule is one that is normally present in a particular cell at a particular developmental stage under particular environmental conditions. Additional endogenous molecules can include proteins.

A “gene,” refers to a DNA region encoding a gene product, as well as all DNA regions which regulate the production of the gene product, whether or not such regulatory sequences are adjacent to coding and/or transcribed sequences. A gene includes, but is not limited to, promoter sequences, enhancers, silencers, insulators, boundary elements, terminators, polyadenylation sequences, post-transcription response elements, translational regulatory sequences such as ribosome binding sites and internal ribosome entry sites, replication origins, matrix attachment sites, and locus control regions.

“Gene expression” refers to the conversion of the information, contained in a gene, into a gene product. A gene product can be the direct transcriptional product of a gene (e.g., mRNA, tRNA, rRNA, antisense RNA, ribozyme, structural RNA or any other type of RNA) or a protein produced by translation of an mRNA. Gene products also include RNAs which are modified, by processes such as capping, polyadenylation, methylation, and editing, and proteins modified by, for example, methylation, acetylation, phosphorylation, ubiquitination, ADP-ribosylation, myristilation, and glycosylation.

As used herein, the term “genetically engineered” or “genetically modified” refers to the chromosomal or extrachromosomal addition of extra genetic material in the form of DNA or RNA to the total genetic material in a cell. Genetic modifications may be targeted or non-targeted to a particular site in a cell's genome. In one embodiment, genetic modification is site-specific. In one embodiment, genetic modification is not site-specific.

As used herein, the term “genome editing” refers to the substitution, deletion, and/or introduction of genetic material at a target site in the cell's genome, which restores, corrects, disrupts, and/or modifies expression of a gene or gene product. Genome editing contemplated in particular embodiments comprises introducing one or more nuclease variants into a cell to generate DNA lesions at or proximal to a target site in the cell's genome, optionally in the presence of a donor repair template.

As used herein, the term “gene therapy” refers to the introduction of extra genetic material into the total genetic material in a cell that restores, corrects, or modifies expression of a gene or gene product, or for the purpose of expressing a therapeutic polypeptide. In particular embodiments, introduction of genetic material into the cell's genome by genome editing that restores, corrects, disrupts, or modifies expression of a gene or gene product, or for the purpose of expressing a therapeutic polypeptide is considered gene therapy.

### C. Nuclease Variants

Nuclease variants contemplated in particular embodiments herein that are suitable for genome editing a target site in the BTK gene comprise one or more DNA binding domains and one or more DNA cleavage domains (e.g., one or more endonuclease and/or exonuclease domains), and optionally, one or more linkers contemplated herein. The terms “reprogrammed nuclease,” “engineered nuclease,” or “nuclease variant” are used interchangeably and refer to a nuclease comprising one or more DNA binding domains and one or more DNA cleavage domains, wherein the nuclease has been designed and/or modified from a parental or naturally occurring nuclease, to bind and cleave a double-stranded DNA target sequence in a BTK gene, preferably a target sequence in the second intron of the human BTK gene, and more preferably a target sequence in the second intron of the human BTK gene as set forth in SEQ ID NO: 24. The nuclease variant may be designed and/or modified from a naturally occurring nuclease or from a previous nuclease variant. Nuclease variants contemplated in particular embodiments may further comprise one or more additional functional domains, e.g., an end-processing enzymatic domain of an end-processing enzyme that exhibits 5′-3′ exonuclease, 5′-3′ alkaline exonuclease, 3′-5′exonuclease (e.g., Trex2), 5′ flap endonuclease, helicase, template-dependent DNA polymerase or template-independent DNA polymerase activity.

Illustrative examples of nuclease variants that bind and cleave a target sequence in the BTK gene include, but are not limited to homing endonuclease variants (meganuclease variants) and megaTALs.

1. Homing Endonuclease (Meganuclease) Variants

In various embodiments, a homing endonuclease or meganuclease is reprogrammed to introduce double-strand breaks (DSBs) in a BTK gene, preferably a target sequence in the second intron of the human BTK gene, and more preferably a target sequence in the second intron of the human BTK gene as set forth in SEQ ID NO: 24. “Homing endonuclease” and “meganuclease” are used interchangeably and refer to naturally-occurring nucleases that recognize 12-45 base-pair cleavage sites and are commonly grouped into five families based on sequence and structure motifs: LAGLIDADG, GIY-YIG, HNH, His-Cys box, and PD-(D/E)XK.

A “reference homing endonuclease” or “reference meganuclease” refers to a wild type homing endonuclease or a homing endonuclease found in nature. In one embodiment, a “reference homing endonuclease” refers to a wild type homing endonuclease that has been modified to increase basal activity.

An “engineered homing endonuclease,” “reprogrammed homing endonuclease,” “homing endonuclease variant,” “engineered meganuclease,” “reprogrammed meganuclease,” or “meganuclease variant” refers to a homing endonuclease comprising one or more DNA binding domains and one or more DNA cleavage domains, wherein the homing endonuclease has been designed and/or modified from a parental or naturally occurring homing endonuclease, to bind and cleave a DNA target sequence in a BTK gene. The homing endonuclease variant may be designed and/or modified from a naturally occurring homing endonuclease or from another homing endonuclease variant. Homing endonuclease variants contemplated in particular embodiments may further comprise one or more additional functional domains, e.g., an end-processing enzymatic domain of an end-processing enzyme that exhibits 5′-3′ exonuclease, 5′-3′ alkaline exonuclease, 3′-5′ exonuclease (e.g., Trex2), 5′ flap endonuclease, helicase, template dependent DNA polymerase or template-independent DNA polymerases activity.

Homing endonuclease (HE) variants do not exist in nature and can be obtained by recombinant DNA technology or by random mutagenesis. HE variants may be obtained by making one or more amino acid alterations, e.g., mutating, substituting, adding, or deleting one or more amino acids, in a naturally occurring HE or HE variant. In particular embodiments, a HE variant comprises one or more amino acid alterations to the DNA recognition interface.

HE variants contemplated in particular embodiments may further comprise one or more linkers and/or additional functional domains, e.g., an end-processing enzymatic domain of an end-processing enzyme that exhibits 5′-3′ exonuclease, 5′-3′ alkaline exonuclease, 3′-5′ exonuclease (e.g., Trex2), 5′ flap endonuclease, helicase, template-dependent DNA polymerase or template-independent DNA polymerases activity. In particular embodiments, HE variants are introduced into a HSC cell with an end-processing enzyme that exhibits 5′-3′ exonuclease, 5′-3′ alkaline exonuclease, 3′-5′ exonuclease (e.g., Trex2), 5′ flap endonuclease, helicase, template-dependent DNA polymerase or template-independent DNA polymerases activity. The HE variant and 3′ processing enzyme may be introduced separately, e.g., in different vectors or separate mRNAs, or together, e.g., as a fusion protein, or in a polycistronic construct separated by a viral self-cleaving peptide or an IRES element.

A “DNA recognition interface” refers to the HE amino acid residues that interact with nucleic acid target bases as well as those residues that are adjacent. For each HE, the DNA recognition interface comprises an extensive network of side chain-to-side chain and side chain-to-DNA contacts, most of which is necessarily unique to recognize a particular nucleic acid target sequence. Thus, the amino acid sequence of the DNA recognition interface corresponding to a particular nucleic acid sequence varies significantly and is a feature of any natural or HE variant. By way of non-limiting example, a HE variant contemplated in particular embodiments may be derived by constructing libraries of HE variants in which one or more amino acid residues localized in the DNA recognition interface of the natural HE (or a previously generated HE variant) are varied. The libraries may be screened for target cleavage activity against each predicted BTK target site using cleavage assays (see e.g., Jarjour et al., 2009. Nuc. Acids Res. 37(20): 6871-6880).

LAGLIDADG homing endonucleases (LHE) are the most well studied family of homing endonucleases, are primarily encoded in archaea and in organellar DNA in green algae and fungi, and display the highest overall DNA recognition specificity. LHEs comprise one or two LAGLIDADG catalytic motifs per protein chain and function as homodimers or single chain monomers, respectively. Structural studies of LAGLIDADG proteins identified a highly conserved core structure (Stoddard 2005), characterized by an αββαββα fold, with the LAGLIDADG motif belonging to the first helix of this fold. The highly efficient and specific cleavage of LHEs represents a protein scaffold to derive novel, highly specific endonucleases. However, engineering LHEs to bind and cleave a non-natural or non-canonical target site requires selection of the appropriate LHE scaffold, examination of the target locus, selection of putative target sites, and extensive alteration of the LHE to alter its DNA contact points and cleavage specificity, at up to two-thirds of the base-pair positions in a target site.

In one embodiment, LHEs from which reprogrammed LHEs or LHE variants may be designed include, but are not limited to I-CreI and I-SceI.

Illustrative examples of LHEs from which reprogrammed LHEs or LHE variants may be designed include, but are not limited to I-AabMI, I-AaeMI, I-AniI, I-ApaMI, I CapIII, I-CapIV, I-CkaMI, I-CpaMI, I-CpaMII, I-CpaMIII, I-CpaMIV, I-CpaMV, I-CpaV, I-CraMI, I-EjeMI, I-GpeMI, I-GpiI, I-GzeMI, I-GzeMII, I-GzeMIII, I-HjeMI, I-LtrII, I-LtrI, I-LtrWI, I-MpeMI, I-MveMI, I-NcrII, I-Ncrl, I-NcrMI, I-OheMI, I-OnuI, I-OsoMI, I-OsoMII, I-OsoMIII, I-OsoMIV, I-PanMI, I-PanMII, I-PanMIII, I-PnoMI, I-ScuMI, I-SmaMI, I-SscMI, and I-Vdi141I.

In one embodiment, the reprogrammed LHE or LHE variant is selected from the group consisting of: an I-CpaMI variant, an I-HjeMI variant, an I-OnuI variant, an I-PanMI variant, and an I-SmaMI variant.

In one embodiment, the reprogrammed LHE or LHE variant is an I-OnuI variant. See e.g., SEQ ID NOs: 6-17.

In one embodiment, reprogrammed I-OnuI LHEs or I-OnuI variants targeting the BTK gene were generated from a natural I-OnuI or biologically active fragment thereof (SEQ ID NOs: 1-5). In a preferred embodiment, reprogrammed I-OnuI LHEs or I-OnuI variants targeting the human BTK gene were generated from an existing I-OnuI variant. In one embodiment, reprogrammed I-OnuI LHEs were generated against a human BTK gene target site set forth in SEQ ID NO: 24.

In a particular embodiment, the reprogrammed I-OnuI LHE or I-OnuI variant that binds and cleaves the human BTK gene comprises one or more amino acid substitutions in the DNA recognition interface. In particular embodiments, the I-OnuI LHE that binds and cleaves the human BTK gene comprises at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity with the DNA recognition interface of I-OnuI (Taekuchi et al. 2011. Proc Natl Acad Sci U.S.A 2011 Aug. 9; 108(32): 13077-13082) or an I-OnuI LHE variant as set forth in SEQ ID NOs: 6-17, or further variants thereof.

In one embodiment, the I-OnuI LHE that binds and cleaves the human BTK gene comprises at least 70%, more preferably at least 80%, more preferably at least 85%, more preferably at least 90%, more preferably at least 95%, more preferably at least 97%, more preferably at least 99% sequence identity with the DNA recognition interface of I-OnuI (Taekuchi et al. 2011. Proc Natl Acad Sci U.S.A 2011 Aug. 9; 108(32): 13077-13082) or an I-OnuI LHE variant as set forth in SEQ ID NOs: 6-17, or further variants thereof.

In a particular embodiment, an I-OnuI LHE variant that binds and cleaves the human BTK gene comprises one or more amino acid substitutions or modifications in the DNA recognition interface of an I-OnuI as set forth in any one of SEQ ID NOs: 1-17, biologically active fragments thereof, and/or further variants thereof.

In a particular embodiment, an I-OnuI LHE variant that binds and cleaves the human BTK gene comprises one or more amino acid substitutions or modifications in the DNA recognition interface, particularly in the subdomains situated from positions 24-50, 68 to 82, 180 to 203 and 223 to 240 of I-OnuI (SEQ ID NOs: 1-5) an I-OnuI variant as set forth in SEQ ID NOs: 6-17, biologically active fragments thereof, and/or further variants thereof.

In a particular embodiment, an I-OnuI LHE that binds and cleaves the human BTK gene comprises one or more amino acid substitutions or modifications in the DNA recognition interface at amino acid positions selected from the group consisting of: 24, 26, 28, 30, 32, 34, 35, 36, 37, 38, 40, 42, 44, 46, 48, 68, 70, 72, 75, 76, 78, 80, 82, 180, 182, 184, 186, 188, 189, 190, 191, 192, 193, 195, 197, 199, 201, 203, 223, 225, 227, 229, 231, 232, 234, 236, 238, and 240 of I-OnuI (SEQ ID NOs: 1-5) or an I-OnuI variant as set forth in SEQ ID NOs: 6-17, biologically active fragments thereof, and/or further variants thereof.

In a particular embodiment, an I-OnuI LHE that binds and cleaves the human BTK gene comprises one or more amino acid substitutions or modifications at amino acid positions selected from the group consisting of: 24, 26, 28, 30, 32, 34, 35, 36, 37, 38, 40, 42, 44, 46, 48, 68, 70, 72, 75, 76, 78, 80, 82, 180, 182, 184, 186, 188, 189, 190, 191, 192, 193, 195, 197, 199, 201, 203, 223, 225, 227, 229, 231, 232, 234, 236, 238, and 240 of I-OnuI (SEQ ID NOs: 1-5) or an I-OnuI variant as set forth in SEQ ID NOs: 6-17, biologically active fragments thereof, and/or further variants thereof.

In a particular embodiment, an I-OnuI LHE that binds and cleaves the human BTK gene comprises 5, 10, 15, 20, 25, 30, 35, or 40 or more amino acid substitutions or modifications in the DNA recognition interface, particularly in the subdomains situated from positions 24-50, 68 to 82, 180 to 203 and 223 to 240 of I-OnuI (SEQ ID NOs: 1-5) or an I-OnuI variant as set forth in SEQ ID NOs: 6-17, biologically active fragments thereof, and/or further variants thereof.

In a particular embodiment, an I-OnuI LHE variant that binds and cleaves the human BTK gene comprises 5, 10, 15, 20, 25, 30, 35, or 40 or more amino acid substitutions or modifications in the DNA recognition interface at amino acid positions selected from the group consisting of: 24, 26, 28, 30, 32, 34, 35, 36, 37, 38, 40, 42, 44, 46, 48, 68, 70, 72, 75, 76, 78, 80, 82, 180, 182, 184, 186, 188, 189, 190, 191, 192, 193, 195, 197, 199, 201, 203, 223, 225, 227, 229, 231, 232, 234, 236, 238, and 240 of I-OnuI SEQ ID NOs: 1-5) or an I-OnuI variant as set forth in SEQ ID NOs: 6-17, biologically active fragments thereof, and/or further variants thereof.

In a particular embodiment, an I-OnuI LHE variant that binds and cleaves the human BTK gene comprises 5, 10, 15, 20, 25, 30, 35, or 40 or more amino acid substitutions or modifications at amino acid positions selected from the group consisting of: 24, 26, 28, 30, 32, 34, 35, 36, 37, 38, 40, 42, 44, 46, 48, 68, 70, 72, 75, 76, 78, 80, 82, 180, 182, 184, 186, 188, 189, 190, 191, 192, 193, 195, 197, 199, 201, 203, 223, 225, 227, 229, 231, 232, 234, 236, 238, and 240 of I-OnuI SEQ ID NOs: 1-5) or an I-OnuI variant as set forth in SEQ ID NOs: 6-17, biologically active fragments thereof, and/or further variants thereof.

In one embodiment, an I-OnuI LHE variant that binds and cleaves the human BTK gene comprises one or more amino acid substitutions or modifications at additional positions situated anywhere within the entire I-OnuI sequence. The residues which may be substituted and/or modified include but are not limited to amino acids that contact the nucleic acid target or that interact with the nucleic acid backbone or with the nucleotide bases, directly or via a water molecule. In one non-limiting example a I-OnuI LHE variant contemplated herein that binds and cleaves the human BTK gene comprises one or more substitutions and/or modifications, preferably at least 5, preferably at least 10, preferably at least 15, preferably at least 20, more preferably at least 25, more preferably at least 30, even more preferably at least 35, or even more preferably at least 40 in at least one position selected from the position group consisting of positions: 24, 26, 28, 30, 32, 34, 35, 36, 37, 38, 40, 42, 44, 46, 48, 61, 68, 70, 72, 75, 76, 78, 80, 82, 85, 116, 135, 138, 143, 147, 159, 164, 168, 178, 180, 182, 184, 186, 188, 189, 190, 191, 192, 193, 195, 197, 199, 201, 203, 210, 223, 225, 227, 229, 231, 232, 234, 236, 238, 240, and 246, of I-OnuI SEQ ID NOs: 1-5) or an I-OnuI variant as set forth in SEQ ID NOs: 6-17, biologically active fragments thereof, and/or further variants thereof.

In particular embodiments, an I-OnuI LHE variant that binds and cleaves the human BTK gene comprises at least 5, at least 15, preferably at least 25, more preferably at least 35, or even more preferably at least 40 or more amino acid substitutions at amino acid positions selected from the group consisting of: 24, 26, 28, 30, 32, 34, 35, 36, 37, 38, 40, 42, 44, 46, 48, 61, 68, 70, 72, 75, 76, 78, 80, 82, 85, 116, 135, 138, 143, 147, 159, 164, 168, 178, 180, 182, 184, 186, 188, 189, 190, 191, 192, 193, 195, 197, 199, 201, 203, 210, 223, 225, 227, 229, 231, 232, 234, 236, 238, 240, and 246 of I-OnuI SEQ ID NOs: 1-5) or an I-OnuI variant as set forth in SEQ ID NOs: 6-17, biologically active fragments thereof, and/or further variants thereof.

In further embodiments, an I-OnuI LHE variant that binds and cleaves the human BTK gene comprises at least 5, at least 15, preferably at least 25, more preferably at least 35, or even more preferably at least 40 or more of the following amino acid substitutions: S24W, L26M, L26S, R28V, R28D, N32S, K34T, S35V, S36K, S40R, E42L, G44S, Q46G, T48E, Q61R, V68K, A70S, A70R, N75H, N75R, A76Y, S78R, K80T, T82S, E85G, V116L, K135R, L138M, T143N, K147E, S159P, I161V, N164S, F168L, E178D, C180S, C180T, F182Y, I186V, S188G, S190N, K191T, L192T, G193R, Q195T, Q195Y, S201Q, S201G, N210Y, K225L, K229V, F232R, W234F, D236Q, V238R, and N246K of I-OnuI SEQ ID NOs: 1-5) or an I-OnuI variant as set forth in SEQ ID NOs: 6-17, biologically active fragments thereof, and/or further variants thereof.

In certain embodiments, an I-OnuI LHE variant that binds and cleaves the human BTK gene comprises the following amino acid substitutions: S24W, R28V, N32S, K34T, S35V, S36K, S40R, E42L, G44S, Q46G, T48E, V68K, A70S, N75H, A76Y, S78R, K80T, T82S, V116L, L138M, T143N, S159P, F168L, E178D, C180S, F182Y, I186V, S188G, S190N, K191T, L192T, G193R, Q195T, S201Q, K225L, K229V, F232R, W234F, D236Q, and V238R of I-OnuI (SEQ ID NOs: 1-5) or an I-OnuI variant as set forth in any one of SEQ ID NOs: 6-17, biologically active fragments thereof, and/or further variants thereof.

In particular embodiments, an I-OnuI LHE variant that binds and cleaves the human BTK gene comprises the following amino acid substitutions: S24W, R28V, N32S, K34T, S35V, S36K, S40R, E42L, G44S, Q46G, T48E, V68K, A70S, N75H, A76Y, S78R, K80T, T82S, V116L, K135R, L138M, T143N, S159P, F168L, E178D, C180S, F182Y, I186V, S188G, S190N, K191T, L192T, G193R, Q195T, S201Q, K225L, K229V, F232R, W234F, D236Q, V238R, and N246K of I-OnuI (SEQ ID NOs: 1-5) or an I-OnuI variant as set forth in any one of SEQ ID NOs: 6-17, biologically active fragments thereof, and/or further variants thereof.

In some embodiments, an I-OnuI LHE variant that binds and cleaves the human BTK gene comprises the following amino acid substitutions: S24W, R28D, N32S, K34T, S35V, S36K, S40R, E42L, G44S, Q46G, T48E, V68K, A70R, N75R, A76Y, K80T, T82S, V116L, L138M, T143N, S159P, N164S, F168L, E178D, C180S, F182Y, I186V, S188G, S190N, K191T, L192T, G193R, Q195T, S201Q, N210Y, K225L, K229V, F232R, W234F, D236Q, and V238R of I-OnuI (SEQ ID NOs: 1-5) or an I-OnuI variant as set forth in any one of SEQ ID NOs: 6-17, biologically active fragments thereof, and/or further variants thereof.

In certain embodiments, an I-OnuI LHE variant that binds and cleaves the human BTK gene comprises the following amino acid substitutions: S24W, R28V, N32S, K34T, S35V, S36K, S40R, E42L, G44S, Q46G, T48E, V68K, A70S, N75H, A76Y, S78R, K80T, T82S, L138M, T143N, S159P, F168L, E178D, C180T, F182Y, S188G, S190N, K191T, L192T, G193R, Q195Y, S201G, K225L, K229V, F232R, W234F, D236Q, and V238R of I-OnuI (SEQ ID NOs: 1-5) or an I-OnuI variant as set forth in any one of SEQ ID NOs: 6-17, biologically active fragments thereof, and/or further variants thereof.

In particular embodiments, an I-OnuI LHE variant that binds and cleaves the human BTK gene comprises the following amino acid substitutions: S24W, R28V, R28D, K34T, S35V, S36K, S40R, E42L, G44S, Q46G, T48E, V68K, A70S, N75H, A76Y, S78R, K80T, T82S, V116L, L138M, T143N, S159P, F168L, E178D, C180S, F182Y, S188G, S190N, K191T, L192T, G193R, Q195T, S201Q, K225L, K229V, F232R, W234F, D236Q, and V238R of I-OnuI (SEQ ID NOs: 1-5) or an I-OnuI variant as set forth in any one of SEQ ID NOs: 6-17, biologically active fragments thereof, and/or further variants thereof.

In additional embodiments, an I-OnuI LHE variant that binds and cleaves the human BTK gene comprises the following amino acid substitutions: S24W, R28D, N32S, K34T, S35V, S36K, S40R, E42L, G44S, Q46G, T48E, V68K, A70S, N75H, A76Y, S78R, K80T, T82S, V116L, L138M, T143N, S159P, F168L, E178D, C180T, F182Y, S188G, S190N, K191T, L192T, G193R, Q195Y, S201G, K225L, K229V, F232R, W234F, D236Q, and V238R of I-OnuI (SEQ ID NOs: 1-5) or an I-OnuI variant as set forth in any one of SEQ ID NOs: 6-17, biologically active fragments thereof, and/or further variants thereof.

In particular embodiments, an I-OnuI LHE variant that binds and cleaves the human BTK gene comprises the following amino acid substitutions: 524W, L26M, R28D, N32S, K34T, S35V, S36K, S40R, E42L, G44S, Q46G, T48E, V68K, A70R, N75R, A76Y, K80T, T82S, V116L, L138M, T143N, K147E, S159P, F168L, E178D, C180T, F182Y, S188G, S190N, K191T, L192T, G193R, Q195Y, S201G, K225L, K229V, F232R, W234F, D236Q, and V238R of I-OnuI (SEQ ID NOs: 1-5) or an I-OnuI variant as set forth in any one of SEQ ID NOs: 6-17, biologically active fragments thereof, and/or further variants thereof.

In certain embodiments, an I-OnuI LHE variant that binds and cleaves the human BTK gene comprises the following amino acid substitutions: S24W, L26S, R28V, N32S, K34T, S35V, S36K, S40R, E42L, G44S, Q46G, T48E, V68K, A70S, N75R, S78R, K80T, E85G, V116L, L138M, T143N, S159P, F168L, E178D, C180T, F182Y, S188G, S190N, K191T, L192T, G193R, Q195Y, S201G, K225L, K229V, F232R, W234F, D236Q, and V238R of I-OnuI (SEQ ID NOs: 1-5) or an I-OnuI variant as set forth in any one of SEQ ID NOs: 6-17, biologically active fragments thereof, and/or further variants thereof.

In particular embodiments, an I-OnuI LHE variant that binds and cleaves the human BTK gene comprises the following amino acid substitutions: S24W, L26S, R28V, N32S, K34T, S35V, S36K, S40R, E42L, G44S, Q46G, T48E, V68K, A70S, N75R, S78R, K80T, E85G, V116L, L138M, T143N, S159P, F168L, E178D, C180T, F182Y, S188G, S190N, K191T, L192T, G193R, Q195Y, S201G, K225L, K229V, F232R, W234F, D236Q, and V238R of I-OnuI (SEQ ID NOs: 1-5) or an I-OnuI variant as set forth in any one of SEQ ID NOs: 6-17, biologically active fragments thereof, and/or further variants thereof.

In some embodiments, an I-OnuI LHE variant that binds and cleaves the human BTK gene comprises the following amino acid substitutions: S24W, R28V, N32S, K34T, S35V, S36K, S40R, E42L, G44S, Q46G, T48E, V68K, A70S, N75H, A76Y, S78R, K80T, T82S, V116L, L138M, T143N, S159P, F168L, E178D, C180T, F182Y, S188G, S190N, K191T, L192T, G193R, Q195Y, S201G, K225L, K229V, F232R, W234F, D236Q, and V238R of I-OnuI (SEQ ID NOs: 1-5) or an I-OnuI variant as set forth in any one of SEQ ID NOs: 6-17, biologically active fragments thereof, and/or further variants thereof.

In certain embodiments, an I-OnuI LHE variant that binds and cleaves the human BTK gene comprises the following amino acid substitutions: S24W, L26S, R28V, N32S, K34T, S35V, S36K, S40R, E42L, G44S, Q46G, T48E, Q61R, V68K, A70S, N75R, S78R, K80T, V116L, L138M, T143N, S159P, F168L, E178D, C180S, F182Y, S188G, S190N, K191T, L192T, G193R, Q195Y, S201G, K225L, K229V, F232R, W234F, D236Q, and V238R of I-OnuI (SEQ ID NOs: 1-5) or an I-OnuI variant as set forth in any one of SEQ ID NOs: 6-17, biologically active fragments thereof, and/or further variants thereof.

In certain embodiments, an I-OnuI LHE variant that binds and cleaves the human BTK gene comprises the following amino acid substitutions: S24W, R28D, N32S, K34T, S35V, S36K, S40R, E42L, G44S, Q46G, T48E, V68K, A70S, N75H, A76Y, S78R, K80T, T82S, V116L, L138M, T143N, K147E, S159P, I161V, F168L, E178D, C180T, F182Y, S188G, S190N, K191T, L192T, G193R, Q195Y, S201G, K225L, K229V, F232R, W234F, D236Q, and V238R of I-OnuI (SEQ ID NOs: 1-5) or an I-OnuI variant as set forth in any one of SEQ ID NOs: 6-17, biologically active fragments thereof, and/or further variants thereof.

In particular embodiments, an I-OnuI LHE variant that binds and cleaves the human BTK gene comprises an amino acid sequence that is at least 80%, preferably at least 85%, more preferably at least 90%, or even more preferably at least 95% identical to the amino acid sequence set forth in any one of SEQ ID NOs: 6-17, or a biologically active fragment thereof.

In particular embodiments, an I-OnuI LHE variant comprises an amino acid sequence set forth in any one of SEQ ID NOs: 6-17, or a biologically active fragment thereof.

In particular embodiments, an I-OnuI LHE variant comprises an amino acid sequence set forth in SEQ ID NO: 6, or a biologically active fragment thereof.

In particular embodiments, an I-OnuI LHE variant comprises an amino acid sequence set forth in SEQ ID NO: 7, or a biologically active fragment thereof.

In particular embodiments, an I-OnuI LHE variant comprises an amino acid sequence set forth in SEQ ID NO: 8, or a biologically active fragment thereof.

In particular embodiments, an I-OnuI LHE variant comprises an amino acid sequence set forth in SEQ ID NO: 9, or a biologically active fragment thereof.

In particular embodiments, an I-OnuI LHE variant comprises an amino acid sequence set forth in SEQ ID NO: 10, or a biologically active fragment thereof.

In particular embodiments, an I-OnuI LHE variant comprises an amino acid sequence set forth in SEQ ID NO: 11, or a biologically active fragment thereof.

In particular embodiments, an I-OnuI LHE variant comprises an amino acid sequence set forth in SEQ ID NO: 12, or a biologically active fragment thereof.

In particular embodiments, an I-OnuI LHE variant comprises an amino acid sequence set forth in SEQ ID NO: 13, or a biologically active fragment thereof.

In particular embodiments, an I-OnuI LHE variant comprises an amino acid sequence set forth in SEQ ID NO: 14, or a biologically active fragment thereof.

In particular embodiments, an I-OnuI LHE variant comprises an amino acid sequence set forth in SEQ ID NO: 15, or a biologically active fragment thereof.

In particular embodiments, an I-OnuI LHE variant comprises an amino acid sequence set forth in SEQ ID NO: 16, or a biologically active fragment thereof.

In particular embodiments, an I-OnuI LHE variant comprises an amino acid sequence set forth in SEQ ID NO: 17, or a biologically active fragment thereof.

In particular embodiments, an I-OnuI LHE variant binds and cleaves the nucleotide sequence set forth in SEQ ID NO: 24 comprises the amino acid sequence set forth in any one of SEQ ID NOs: 6 to 17.

2. MegaTALs

In various embodiments, a megaTAL comprising a homing endonuclease variant is reprogrammed to introduce double-strand breaks (DSBs) in a BTK gene, preferably a target sequence in the second intron of the human BTK gene, and more preferably a target sequence in the second intron of the human BTK gene as set forth in SEQ ID NO: 24. A “megaTAL” refers to a polypeptide comprising a TALE DNA binding domain and a homing endonuclease variant that binds and cleaves a DNA target sequence in a BTK gene, and optionally comprises one or more linkers and/or additional functional domains, e.g., an end-processing enzymatic domain of an end-processing enzyme that exhibits 5′-3′ exonuclease, 5′-3′ alkaline exonuclease, 3′-5′ exonuclease (e.g., Trex2), 5′ flap endonuclease, helicase or template-independent DNA polymerases activity.

In particular embodiments, a megaTAL can be introduced into a cell along with an end-processing enzyme that exhibits 5′-3′ exonuclease, 5′-3′ alkaline exonuclease, 3′-5′ exonuclease (e.g., Trex2), 5′ flap endonuclease, helicase, template-dependent DNA polymerase or template-independent DNA polymerase activity. The megaTAL and 3′ processing enzyme may be introduced separately, e.g., in different vectors or separate mRNAs, or together, e.g., as a fusion protein, or in a polycistronic construct separated by a viral self-cleaving peptide or an IRES element.

A “TALE DNA binding domain” is the DNA binding portion of transcription activator-like effectors (TALE or TAL-effectors), which mimics plant transcriptional activators to manipulate the plant transcriptome (see e.g., Kay et al., 2007. Science 318:648-651). TALE DNA binding domains contemplated in particular embodiments are engineered de novo or from naturally occurring TALEs, e.g., AvrBs3 from Xanthomonas campestris pv. vesicatoria, Xanthomonas gardneri, Xanthomonas translucens, Xanthomonas axonopodis, Xanthomonas perforans, Xanthomonas alfalfa, Xanthomonas citri, Xanthomonas euvesicatoria, and Xanthomonas oryzae and brg11 and hpx17 from Ralstonia solanacearum. Illustrative examples of TALE proteins for deriving and designing DNA binding domains are disclosed in U.S. Pat. No. 9,017,967, and references cited therein, all of which are incorporated herein by reference in their entireties.

In particular embodiments, a megaTAL comprises a TALE DNA binding domain comprising one or more repeat units that are involved in binding of the TALE DNA binding domain to its corresponding target DNA sequence. A single “repeat unit” (also referred to as a “repeat”) is typically 33-35 amino acids in length. Each TALE DNA binding domain repeat unit includes 1 or 2 DNA-binding residues making up the Repeat Variable Di-Residue (RVD), typically at positions 12 and/or 13 of the repeat. The natural (canonical) code for DNA recognition of these TALE DNA binding domains has been determined such that an HD sequence at positions 12 and 13 leads to a binding to cytosine (C), NG binds to T, NI to A, NN binds to G or A, and NG binds to T. In certain embodiments, non-canonical (atypical) RVDs are contemplated.

Illustrative examples of non-canonical RVDs suitable for use in particular megaTALs contemplated in particular embodiments include, but are not limited to HH, KH, NH, NK, NQ, RH, RN, SS, NN, SN, KN for recognition of guanine (G); NI, KI, RI, HI, SI for recognition of adenine (A); NG, HG, KG, RG for recognition of thymine (T); RD, SD, HD, ND, KD, YG for recognition of cytosine (C); NV, HN for recognition of A or G; and H*, HA, KA, N*, NA, NC, NS, RA, S*for recognition of A or T or G or C, wherein (*) means that the amino acid at position 13 is absent. Additional illustrative examples of RVDs suitable for use in particular megaTALs contemplated in particular embodiments further include those disclosed in U.S. Pat. No. 8,614,092, which is incorporated herein by reference in its entirety.

In particular embodiments, a megaTAL contemplated herein comprises a TALE DNA binding domain comprising 3 to 30 repeat units. In certain embodiments, a megaTAL comprises 3, 4, 5, 6, 7, 8, 9, 10, 11, 12, 13, 14, 15, 16, 17, 18, 19, 20, 21, 22, 23, 24, 25, 26, 27, 28, 29, or 30 TALE DNA binding domain repeat units. In a preferred embodiment, a megaTAL contemplated herein comprises a TALE DNA binding domain comprising 5-15 repeat units, more preferably 7-15 repeat units, more preferably 9-15 repeat units, and more preferably 9, 10, 11, 12, 13, 14, or 15 repeat units.

In particular embodiments, a megaTAL contemplated herein comprises a TALE DNA binding domain comprising 3 to 30 repeat units and an additional single truncated TALE repeat unit comprising 20 amino acids located at the C-terminus of a set of TALE repeat units, i.e., an additional C-terminal half-TALE DNA binding domain repeat unit (amino acids −20 to −1 of the C-cap disclosed elsewhere herein, infra). Thus, in particular embodiments, a megaTAL contemplated herein comprises a TALE DNA binding domain comprising 3.5 to 30.5 repeat units. In certain embodiments, a megaTAL comprises 3.5, 4.5, 5.5, 6.5, 7.5, 8.5, 9.5, 10.5, 11.5, 12.5, 13.5, 14.5, 15.5, 16.5, 17.5, 18.5, 19.5, 20.5, 21.5, 22.5, 23.5, 24.5, 25.5, 26.5, 27.5, 28.5, 29.5, or 30.5 TALE DNA binding domain repeat units. In a preferred embodiment, a megaTAL contemplated herein comprises a TALE DNA binding domain comprising 5.5-15.5 repeat units, more preferably 7.5-15.5 repeat units, more preferably 9.5-15.5 repeat units, and more preferably 9.5, 10.5, 11.5, 12.5, 13.5, 14.5, or 15.5 repeat units.

In particular embodiments, a megaTAL comprises a TAL effector architecture comprising an “N-terminal domain (NTD)” polypeptide, one or more TALE repeat domains/units, a “C-terminal domain (CTD)” polypeptide, and a homing endonuclease variant. In some embodiments, the NTD, TALE repeats, and/or CTD domains are from the same species. In other embodiments, one or more of the NTD, TALE repeats, and/or CTD domains are from different species.

As used herein, the term “N-terminal domain (NTD)” polypeptide refers to the sequence that flanks the N-terminal portion or fragment of a naturally occurring TALE DNA binding domain. The NTD sequence, if present, may be of any length as long as the TALE DNA binding domain repeat units retain the ability to bind DNA. In particular embodiments, the NTD polypeptide comprises at least 120 to at least 140 or more amino acids N-terminal to the TALE DNA binding domain (0 is amino acid 1 of the most N-terminal repeat unit). In particular embodiments, the NTD polypeptide comprises at least about 120, 121, 122, 123, 124, 125, 126, 127, 128, 129, 130, 131, 132, 133, 134, 135, 136, 137, 138, 139, or at least 140 amino acids N-terminal to the TALE DNA binding domain. In one embodiment, a megaTAL contemplated herein comprises an NTD polypeptide of at least about amino acids +1 to +122 to at least about +1 to +137 of a Xanthomonas TALE protein (0 is amino acid 1 of the most N-terminal repeat unit). In particular embodiments, the NTD polypeptide comprises at least about 122, 123, 124, 125, 126, 127, 128, 129, 130, 131, 132, 133, 134, 135, 136, or 137 amino acids N-terminal to the TALE DNA binding domain of a Xanthomonas TALE protein. In one embodiment, a megaTAL contemplated herein comprises an NTD polypeptide of at least amino acids +1 to +121 of a Ralstonia TALE protein (0 is amino acid 1 of the most N-terminal repeat unit). In particular embodiments, the NTD polypeptide comprises at least about 121, 122, 123, 124, 125, 126, 127, 128, 129, 130, 131, 132, 133, 134, 135, 136, or 137 amino acids N-terminal to the TALE DNA binding domain of a Ralstonia TALE protein.

As used herein, the term “C-terminal domain (CTD)” polypeptide refers to the sequence that flanks the C-terminal portion or fragment of a naturally occurring TALE DNA binding domain. The CTD sequence, if present, may be of any length as long as the TALE DNA binding domain repeat units retain the ability to bind DNA. In particular embodiments, the CTD polypeptide comprises at least 20 to at least 85 or more amino acids C-terminal to the last full repeat of the TALE DNA binding domain (the first 20 amino acids are the half-repeat unit C-terminal to the last C-terminal full repeat unit). In particular embodiments, the CTD polypeptide comprises at least about 20, 21, 22, 23, 24, 25, 26, 27, 28, 29, 30, 31, 32, 33, 34, 35, 36, 37, 38, 39, 40, 41, 42, 443, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, or at least 85 amino acids C-terminal to the last full repeat of the TALE DNA binding domain. In one embodiment, a megaTAL contemplated herein comprises a CTD polypeptide of at least about amino acids −20 to −1 of a Xanthomonas TALE protein (−20 is amino acid 1 of a half-repeat unit C-terminal to the last C-terminal full repeat unit). In particular embodiments, the CTD polypeptide comprises at least about 20, 19, 18, 17, 16, 15, 14, 13, 12, 11, 10, 9, 8, 7, 6, 5, 4, 3, 2, or 1 amino acids C-terminal to the last full repeat of the TALE DNA binding domain of a Xanthomonas TALE protein. In one embodiment, a megaTAL contemplated herein comprises a CTD polypeptide of at least about amino acids −20 to −1 of a Ralstonia TALE protein (−20 is amino acid 1 of a half-repeat unit C-terminal to the last C-terminal full repeat unit). In particular embodiments, the CTD polypeptide comprises at least about 20, 19, 18, 17, 16, 15, 14, 13, 12, 11, 10, 9, 8, 7, 6, 5, 4, 3, 2, or 1 amino acids C-terminal to the last full repeat of the TALE DNA binding domain of a Ralstonia TALE protein.

In particular embodiments, a megaTAL contemplated herein, comprises a fusion polypeptide comprising a TALE DNA binding domain engineered to bind a target sequence, a homing endonuclease reprogrammed to bind and cleave a target sequence, and optionally an NTD and/or CTD polypeptide, optionally joined to each other with one or more linker polypeptides contemplated elsewhere herein. Without wishing to be bound by any particular theory, it is contemplated that a megaTAL comprising TALE DNA binding domain, and optionally an NTD and/or CTD polypeptide is fused to a linker polypeptide which is further fused to a homing endonuclease variant. Thus, the TALE DNA binding domain binds a DNA target sequence that is within about 1, 2, 3, 4, 5, 6, 7, 8, 9, 10, 11, 12, 13, 14, or 15 nucleotides away from the target sequence bound by the DNA binding domain of the homing endonuclease variant. In this way, the megaTALs contemplated herein, increase the specificity and efficiency of genome editing.

In one embodiment, a megaTAL comprises a homing endonuclease variant and a TALE DNA binding domain that binds a nucleotide sequence that is within about 4, 5, or 6 nucleotides, preferably, 6 nucleotides upstream of the binding site of the reprogrammed homing endonuclease.

In one embodiment, a megaTAL comprises a homing endonuclease variant and a TALE DNA binding domain that binds the nucleotide sequence set forth in SEQ ID NO: 25, which is 6 nucleotides upstream of the nucleotide sequence bound and cleaved by the homing endonuclease variant (SEQ ID NO: 24). In preferred embodiments, the megaTAL target sequence is SEQ ID NO: 26.

In particular embodiments, a megaTAL contemplated herein, comprises one or more TALE DNA binding repeat units and an LHE variant designed or reprogrammed from an LHE selected from the group consisting of: I-AabMI, I-AaeMI, I-AniI, I-ApaMI, I-CapIII, I-CapIV, I-CkaMI, I-CpaMI, I-CpaMII, I-CpaMIII, I-CpaMIV, I-CpaMV, I-CpaV, I-CraMI, I-EjeMI, I-GpeMI, I-GpiI, I-GzeMI, I-GzeMII, I-GzeMIII, I-HjeMI, I-LtrII, I-LtrI, I-LtrWI, I-MpeMI, I-MveMI, I-NcrII, I-Ncrl, I-NcrMI, I-OheMI, I-OnuI, I-OsoMI, I-OsoMII, I-OsoMIII, I-OsoMIV, I-PanMI, I-PanMII, I-PanMIII, I-PnoMI, I-ScuMI, I-SmaMI, I-SscMI, I-Vdi141I and variants thereof, or preferably I-CpaMI, I-HjeMI, I-OnuI, I-PanMI, SmaMI and variants thereof, or more preferably I-OnuI and variants thereof.

In particular embodiments, a megaTAL contemplated herein, comprises an NTD, one or more TALE DNA binding repeat units, a CTD, and an LHE variant selected from the group consisting of: I-AabMI, I-AaeMI, I-AniI, I-ApaMI, I-CapIII, I-CapIV, I-CkaMI, I-CpaMI, I-CpaMII, I-CpaMIII, I-CpaMIV, I-CpaMV, I-CpaV, I-CraMI, I-EjeMI, I-GpeMI, I-GpiI, I-GzeMI, I-GzeMII, I-GzeMIII, I-HjeMI, I-LtrII, I-LtrI, I-LtrWI, I-MpeMI, I-MveMI, I-NcrII, I-Ncrl, I-NcrMI, I-OheMI, I-OnuI, I-OsoMI, I-OsoMII, I-OsoMIII, I-OsoMIV, I-PanMI, I-PanMII, I-PanMIII, I-PnoMI, I-ScuMI, I-SmaMI, I-SscMI, I-Vdi141I and variants thereof, or preferably I-CpaMI, I-HjeMI, I-OnuI, I-PanMI, SmaMI and variants thereof, or more preferably I-OnuI and variants thereof.

In particular embodiments, a megaTAL contemplated herein, comprises an NTD, about 9.5 to about 15.5 TALE DNA binding repeat units, and an LHE variant selected from the group consisting of: I-AabMI, I-AaeMI, I-AniI, I-ApaMI, I-CapIII, I-CapIV, I-CkaMI, I-CpaMI, I-CpaMII, I-CpaMIII, I-CpaMIV, I-CpaMV, I-CpaV, I-CraMI, I-EjeMI, I-GpeMI, I-GpiI, I-GzeMI, I-GzeMII, I-GzeMIII, I-HjeMI, I-LtrII, I-LtrI, I-LtrWI, I-MpeMI, I-MveMI, I-NcrII, I-Ncrl, I-NcrMI, I-OheMI, I-OnuI, I-OsoMI, I-OsoMII, I-OsoMIII, I-OsoMIV, I-PanMI, I-PanMII, I-PanMIII, I-PnoMI, I-ScuMI, I-SmaMI, I-SscMI, I-Vdi141I and variants thereof, or preferably I-CpaMI, I-HjeMI, I-OnuI, I-PanMI, SmaMI and variants thereof, or more preferably I-OnuI and variants thereof.

In particular embodiments, a megaTAL contemplated herein, comprises an NTD of about 122 amino acids to 137 amino acids, about 9.5, about 10.5, about 11.5, about 12.5, about 13.5, about 14.5, or about 15.5 binding repeat units, a CTD of about 20 amino acids to about 85 amino acids, and an I-OnuI LHE variant. In particular embodiments, any one of, two of, or all of the NTD, DNA binding domain, and CTD can be designed from the same species or different species, in any suitable combination.

In particular embodiments, a megaTAL contemplated herein, comprises the amino acid sequence set forth in any one of SEQ ID NOs: 18 to 20.

In particular embodiments, a megaTAL-Trex2 fusion protein contemplated herein, comprises the amino acid sequence set forth in any one of SEQ ID NO: 21 to 23.

In certain embodiments, a megaTAL contemplated herein, is encoded by an mRNA sequence set forth in any one of SEQ ID NO: 27 to 29.

In certain embodiments, a megaTAL comprises a TALE DNA binding domain and an I-OnuI LHE variant binds and cleaves the nucleotide sequence set forth in SEQ ID NO: 26.

In particular embodiments, a megaTAL comprises a TALE DNA binding domain and an I-OnuI LHE variant binds and cleaves the nucleotide sequence set forth in SEQ ID NO: 26 comprises the amino acid sequence set forth in any one of SEQ ID NOs: 18 to 20.

3. End-Processing Enzymes

Genome editing compositions and methods contemplated in particular embodiments comprise editing cellular genomes using a nuclease variant and an end-processing enzyme. In particular embodiments, a single polynucleotide encodes a homing endonuclease variant and an end-processing enzyme, separated by a linker, a self-cleaving peptide sequence, e.g., 2A sequence, or by an IRES sequence. In particular embodiments, genome editing compositions comprise a polynucleotide encoding a nuclease variant and a separate polynucleotide encoding an end-processing enzyme.

The term “end-processing enzyme” refers to an enzyme that modifies the exposed ends of a polynucleotide chain. The polynucleotide may be double-stranded DNA (dsDNA), single-stranded DNA (ssDNA), RNA, double-stranded hybrids of DNA and RNA, and synthetic DNA (for example, containing bases other than A, C, G, and T). An end-processing enzyme may modify exposed polynucleotide chain ends by adding one or more nucleotides, removing one or more nucleotides, removing or modifying a phosphate group and/or removing or modifying a hydroxyl group. An end-processing enzyme may modify ends at endonuclease cut sites or at ends generated by other chemical or mechanical means, such as shearing (for example by passing through fine-gauge needle, heating, sonicating, mini bead tumbling, and nebulizing), ionizing radiation, ultraviolet radiation, oxygen radicals, chemical hydrolysis and chemotherapy agents.

In particular embodiments, genome editing compositions and methods contemplated in particular embodiments comprise editing cellular genomes using a homing endonuclease variant or megaTAL and a DNA end-processing enzyme.

The term “DNA end-processing enzyme” refers to an enzyme that modifies the exposed ends of DNA. A DNA end-processing enzyme may modify blunt ends or staggered ends (ends with 5′ or 3′ overhangs). A DNA end-processing enzyme may modify single stranded or double stranded DNA. A DNA end-processing enzyme may modify ends at endonuclease cut sites or at ends generated by other chemical or mechanical means, such as shearing (for example by passing through fine-gauge needle, heating, sonicating, mini bead tumbling, and nebulizing), ionizing radiation, ultraviolet radiation, oxygen radicals, chemical hydrolysis and chemotherapy agents. DNA end-processing enzyme may modify exposed DNA ends by adding one or more nucleotides, removing one or more nucleotides, removing or modifying a phosphate group and/or removing or modifying a hydroxyl group.

Illustrative examples of DNA end-processing enzymes suitable for use in particular embodiments contemplated herein include, but are not limited to: 5′-3′ exonucleases, 5′-3′ alkaline exonucleases, 3′-5′ exonucleases, 5′ flap endonucleases, helicases, phosphatases, hydrolases and template-independent DNA polymerases.

Additional illustrative examples of DNA end-processing enzymes suitable for use in particular embodiments contemplated herein include, but are not limited to, Trex2, Trex1, Trex1 without transmembrane domain, Apollo, Artemis, DNA2, Exo1, ExoT, ExoIII, Fen1, Fan1, MreII, Rad2, Rad9, TdT (terminal deoxynucleotidyl transferase), PNKP, RecE, RecJ, RecQ, Lambda exonuclease, Sox, Vaccinia DNA polymerase, exonuclease I, exonuclease III, exonuclease VII, NDK1, NDK5, NDK7, NDK8, WRN, T7 exonuclease Gene 6, avian myeloblastosis virus integration protein (IN), Bloom, Antartic Phophatase, Alkaline Phosphatase, Poly nucleotide Kinase (PNK), ApeI, Mung Bean nuclease, Hex1, TTRAP (TDP2), Sgs1, Sae2, CUP, Pol mu, Pol lambda, MUS81, EME1, EME2, SLX1, SLX4 and UL-12.

In particular embodiments, genome editing compositions and methods for editing cellular genomes contemplated herein comprise polypeptides comprising a homing endonuclease variant or megaTAL and an exonuclease. The term “exonuclease” refers to enzymes that cleave phosphodiester bonds at the end of a polynucleotide chain via a hydrolyzing reaction that breaks phosphodiester bonds at either the 3′ or 5′ end.

Illustrative examples of exonucleases suitable for use in particular embodiments contemplated herein include, but are not limited to: hExoI, Yeast ExoI, E. coli ExoI, hTREX2, mouse TREX2, rat TREX2, hTREX1, mouse TREX1, rat TREX1, and Rat TREX1.

In particular embodiments, the DNA end-processing enzyme is a 3′ or 5′ exonuclease, preferably Trex 1 or Trex2, more preferably Trex2, and even more preferably human or mouse Trex2.

### D. Target Sites

Nuclease variants contemplated in particular embodiments can be designed to bind to any suitable target sequence in a BTK gene and can have a novel binding specificity, compared to a naturally-occurring nuclease. In particular embodiments, the target site is a regulatory region of a gene including, but not limited to promoters, enhancers, repressor elements, and the like. In particular embodiments, the target site is a coding region of a gene or a splice site. In particular embodiments, a nuclease variant and donor repair template can be designed to insert a therapeutic polynucleotide. In particular embodiments, a nuclease variant and donor repair template can be designed to insert a therapeutic polynucleotide under control of the endogenous BTK gene regulatory elements or expression control sequences.

In various embodiments, nuclease variants bind to and cleave a target sequence in the Bruton's tyrosine kinase (BTK) gene, which is located on the X chromosome. The BTK gene encodes a tyrosine kinase, which is essential for the development and maturation of B cells. BTK is also referred to as Bruton Agammaglobulinemia Tyrosine Kinase, B-Cell Progenitor Kinase (BPK), Tyrosine-Protein Kinase BTK Isoform (Lacking Exon 13 To 17), Dominant-Negative Kinase-Deficient Brutons Tyrosine Kinase, Tyrosine-Protein Kinase BTK Isoform (Lacking Exon 14), Truncated Bruton Agammaglobulinemia Tyrosine Kinase, PSCTK1, AGMX1, Agammaglobulinaemia Tyrosine Kinase (ATK), Agammaglobulinemia Tyrosine Kinase, Tyrosine-Protein Kinase BTK, and IMD1, among others. Exemplary BTK reference sequences numbers used in particular embodiments include, but are not limited to NM_000061.2, NP_000052.1, AK057105, BC109079, DA619542, DB636737, CCDS14482.1, Q06187, Q5JY90, ENSP00000308176.7, OTTHUMP00000023676, ENST00000308731.7, OTTHUMT00000057532, NM_001287344.1, NP_001274273.1, NM_001287345.1, and NP_001274274.1.

In particular embodiments, a homing endonuclease variant or megaTAL introduces a double-strand break (DSB) in a BTK gene, preferably a target sequence in the second intron of the human BTK gene, and more preferably a target sequence in the second intron of the human BTK gene as set forth in SEQ ID NO: 24. In particular embodiments, the reprogrammed nuclease or megaTAL comprises an I-OnuI LHE variant that introduces a double strand break at the target site in the second intron of the BTK gene as set forth in SEQ ID NO: 24 by cleaving the sequence “ACTT.”

In a preferred embodiment, a homing endonuclease variant or megaTAL is cleaves double-stranded DNA and introduces a DSB into the polynucleotide sequence set forth in SEQ ID NO: 24 or 26.

In a preferred embodiment, the BTK gene is a human BTK gene.

### E. Donor Repair Templates

Nuclease variants may be used to introduce a DSB in a target sequence; the DSB may be repaired through homology directed repair (HDR) mechanisms in the presence of one or more donor repair templates. In particular embodiments, the donor repair template is used to insert a sequence into the genome. In particular preferred embodiments, the donor repair template is used to insert a polynucleotide sequence encoding a therapeutic BTK polypeptide, e.g., SEQ ID N: 32. In particular preferred embodiments, the donor repair template is used to insert a polynucleotide sequence encoding a therapeutic BTK polypeptide, such that the expression of the BTK polypeptide is under control of the endogenous BTK promoter and/or enhancers.

In various embodiments, a donor repair template is introduced into a hematopoietic cell, e.g., a hematopoietic stem or progenitor cell, or CD34⁺ cell, by transducing the cell with an adeno-associated virus (AAV), retrovirus, e.g., lentivirus, IDLV, etc., herpes simplex virus, adenovirus, or vaccinia virus vector comprising the donor repair template.

In particular embodiments, the donor repair template comprises one or more homology arms that flank the DSB site.

As used herein, the term “homology arms” refers to a nucleic acid sequence in a donor repair template that is identical, or nearly identical, to DNA sequence flanking the DNA break introduced by the nuclease at a target site. In one embodiment, the donor repair template comprises a 5′ homology arm that comprises a nucleic acid sequence that is identical or nearly identical to the DNA sequence 5′ of the DNA break site. In one embodiment, the donor repair template comprises a 3′ homology arm that comprises a nucleic acid sequence that is identical or nearly identical to the DNA sequence 3′ of the DNA break site. In a preferred embodiment, the donor repair template comprises a 5′ homology arm and a 3′ homology arm. The donor repair template may comprise homology to the genome sequence immediately adjacent to the DSB site, or homology to the genomic sequence within any number of base pairs from the DSB site. In one embodiment, the donor repair template comprises a nucleic acid sequence that is homologous to a genomic sequence about 5 bp, about 10 bp, about 25 bp, about 50 bp, about 100 bp, about 250 bp, about 500 bp, about 1000 bp, about 2500 bp, about 5000 bp, about 10000 bp or more, including any intervening length of homologous sequence.

Illustrative examples of suitable lengths of homology arms contemplated in particular embodiments, may be independently selected, and include but are not limited to: about 100 bp, about 200 bp, about 300 bp, about 400 bp, about 500 bp, about 600 bp, about 700 bp, about 800 bp, about 900 bp, about 1000 bp, about 1100 bp, about 1200 bp, about 1300 bp, about 1400 bp, about 1500 bp, about 1600 bp, about 1700 bp, about 1800 bp, about 1900 bp, about 2000 bp, about 2100 bp, about 2200 bp, about 2300 bp, about 2400 bp, about 2500 bp, about 2600 bp, about 2700 bp, about 2800 bp, about 2900 bp, or about 3000 bp, or longer homology arms, including all intervening lengths of homology arms.

Additional illustrative examples of suitable homology arm lengths include, but are not limited to: about 100 bp to about 3000 bp, about 200 bp to about 3000 bp, about 300 bp to about 3000 bp, about 400 bp to about 3000 bp, about 500 bp to about 3000 bp, about 500 bp to about 2500 bp, about 500 bp to about 2000 bp, about 750 bp to about 2000 bp, about 750 bp to about 1500 bp, or about 1000 bp to about 1500 bp, including all intervening lengths of homology arms.

In a particular embodiment, the lengths of the 5′ and 3′ homology arms are independently selected from about 500 bp to about 1500 bp. In one embodiment, the 5′homology arm is about 1500 bp and the 3′ homology arm is about 1000 bp. In one embodiment, the 5′homology arm is between about 200 bp to about 600 bp and the 3′ homology arm is between about 200 bp to about 600 bp. In one embodiment, the 5′homology arm is about 200 bp and the 3′ homology arm is about 200 bp. In one embodiment, the 5′homology arm is about 300 bp and the 3′ homology arm is about 300 bp. In one embodiment, the 5′homology arm is about 400 bp and the 3′ homology arm is about 400 bp. In one embodiment, the 5′homology arm is about 500 bp and the 3′ homology arm is about 500 bp. In one embodiment, the 5′homology arm is about 600 bp and the 3′ homology arm is about 600 bp.

### F. Polypeptides

Various polypeptides are contemplated herein, including, but not limited to, homing endonuclease variants, megaTALs, and fusion polypeptides. In preferred embodiments, a polypeptide comprises the amino acid sequence set forth in SEQ ID NOs: 1-23 and 31-32. “Polypeptide,” “polypeptide fragment,” “peptide” and “protein” are used interchangeably, unless specified to the contrary, and according to conventional meaning, i.e., as a sequence of amino acids. In one embodiment, a “polypeptide” includes fusion polypeptides and other variants. Polypeptides can be prepared using any of a variety of well-known recombinant and/or synthetic techniques. Polypeptides are not limited to a specific length, e.g., they may comprise a full-length protein sequence, a fragment of a full length protein, or a fusion protein, and may include post-translational modifications of the polypeptide, for example, glycosylations, acetylations, phosphorylations and the like, as well as other modifications known in the art, both naturally occurring and non-naturally occurring.

An “isolated protein,” “isolated peptide,” or “isolated polypeptide” and the like, as used herein, refer to in vitro synthesis, isolation, and/or purification of a peptide or polypeptide molecule from a cellular environment, and from association with other components of the cell, i.e., it is not significantly associated with in vivo substances.

Illustrative examples of polypeptides contemplated in particular embodiments include, but are not limited to homing endonuclease variants, megaTALs, end-processing nucleases, fusion polypeptides and variants thereof.

Polypeptides include “polypeptide variants.” Polypeptide variants may differ from a naturally occurring polypeptide in one or more amino acid substitutions, deletions, additions and/or insertions. Such variants may be naturally occurring or may be synthetically generated, for example, by modifying one or more amino acids of the above polypeptide sequences. For example, in particular embodiments, it may be desirable to improve the biological properties of a homing endonuclease, megaTAL or the like that binds and cleaves a target site in the human BTK gene by introducing one or more substitutions, deletions, additions and/or insertions into the polypeptide. In particular embodiments, polypeptides include polypeptides having at least about 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% amino acid identity to any of the reference sequences contemplated herein, typically where the variant maintains at least one biological activity of the reference sequence.

Polypeptides variants include biologically active “polypeptide fragments.” Illustrative examples of biologically active polypeptide fragments include DNA binding domains, nuclease domains, and the like. As used herein, the term “biologically active fragment” or “minimal biologically active fragment” refers to a polypeptide fragment that retains at least 100%, at least 90%, at least 80%, at least 70%, at least 60%, at least 50%, at least 40%, at least 30%, at least 20%, at least 10%, or at least 5% of the naturally occurring polypeptide activity. In preferred embodiments, the biological activity is binding affinity and/or cleavage activity for a target sequence. In certain embodiments, a polypeptide fragment can comprise an amino acid chain at least 5 to about 1700 amino acids long. It will be appreciated that in certain embodiments, fragments are at least 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60, 65, 70, 75, 80, 85, 90, 95, 100, 110, 150, 200, 250, 300, 350, 400, 450, 500, 550, 600, 650, 700, 750, 800, 850, 900, 950, 1000, 1100, 1200, 1300, 1400, 1500, 1600, 1700 or more amino acids long. In particular embodiments, a polypeptide comprises a biologically active fragment of a homing endonuclease variant. In particular embodiments, the polypeptides set forth herein may comprise one or more amino acids denoted as “X.” “X” if present in an amino acid SEQ ID NO, refers to any amino acid. One or more “X” residues may be present at the N- and C-terminus of an amino acid sequence set forth in particular SEQ ID NOs contemplated herein. If the “X” amino acids are not present the remaining amino acid sequence set forth in a SEQ ID NO may be considered a biologically active fragment.

In particular embodiments, a polypeptide comprises a biologically active fragment of a homing endonuclease variant, e.g., SEQ ID NOs: 6-17 or a megaTAL (SEQ ID NOs: 18-20). The biologically active fragment may comprise an N-terminal truncation and/or C-terminal truncation. In a particular embodiment, a biologically active fragment lacks or comprises a deletion of the 1, 2, 3, 4, 5, 6, 7, or 8 N-terminal amino acids of a homing endonuclease variant compared to a corresponding wild type homing endonuclease sequence, more preferably a deletion of the 4 N-terminal amino acids of a homing endonuclease variant compared to a corresponding wild type homing endonuclease sequence. In a particular embodiment, a biologically active fragment lacks or comprises a deletion of the 1, 2, 3, 4, or 5 C-terminal amino acids of a homing endonuclease variant compared to a corresponding wild type homing endonuclease sequence, more preferably a deletion of the 2 C-terminal amino acids of a homing endonuclease variant compared to a corresponding wild type homing endonuclease sequence. In a particular preferred embodiment, a biologically active fragment lacks or comprises a deletion of the 4 N-terminal amino acids and 2 C-terminal amino acids of a homing endonuclease variant compared to a corresponding wild type homing endonuclease sequence.

In a particular embodiment, an I-OnuI variant comprises a deletion of 1, 2, 3, 4, 5, 6, 7, or 8 the following N-terminal amino acids: M, A, Y, M, S, R, R, E; and/or a deletion of the following 1, 2, 3, 4, or 5 C-terminal amino acids: R, G, S, F, V.

In a particular embodiment, an I-OnuI variant comprises a deletion or substitution of 1, 2, 3, 4, 5, 6, 7, or 8 the following N-terminal amino acids: M, A, Y, M, S, R, R, E; and/or a deletion or substitution of the following 1, 2, 3, 4, or 5 C-terminal amino acids: R, G, S, F, V.

In a particular embodiment, an I-OnuI variant comprises a deletion of 1, 2, 3, 4, 5, 6, 7, or 8 the following N-terminal amino acids: M, A, Y, M, S, R, R, E; and/or a deletion of the following 1 or 2 C-terminal amino acids: F, V.

In a particular embodiment, an I-OnuI variant comprises a deletion or substitution of 1, 2, 3, 4, 5, 6, 7, or 8 the following N-terminal amino acids: M, A, Y, M, S, R, R, E; and/or a deletion or substitution of the following 1 or 2 C-terminal amino acids: F, V.

As noted above, polypeptides may be altered in various ways including amino acid substitutions, deletions, truncations, and insertions. Methods for such manipulations are generally known in the art. For example, amino acid sequence variants of a reference polypeptide can be prepared by mutations in the DNA. Methods for mutagenesis and nucleotide sequence alterations are well known in the art. See, for example, Kunkel (1985, Proc. Natl. Acad. Sci. USA. 82: 488-492), Kunkel et al., (1987, Methods in Enzymol, 154: 367-382), U.S. Pat. No. 4,873,192, Watson, J. D. et al., (Molecular Biology of the Gene, Fourth Edition, Benjamin/Cummings, Menlo Park, Calif., 1987) and the references cited therein. Guidance as to appropriate amino acid substitutions that do not affect biological activity of the protein of interest may be found in the model of Dayhoff et al., (1978) Atlas of Protein Sequence and Structure (Natl. Biomed. Res. Found, Washington, D.C.).

In certain embodiments, a variant will contain one or more conservative substitutions. A “conservative substitution” is one in which an amino acid is substituted for another amino acid that has similar properties, such that one skilled in the art of peptide chemistry would expect the secondary structure and hydropathic nature of the polypeptide to be substantially unchanged. Modifications may be made in the structure of the polynucleotides and polypeptides contemplated in particular embodiments, polypeptides include polypeptides having at least about and still obtain a functional molecule that encodes a variant or derivative polypeptide with desirable characteristics. When it is desired to alter the amino acid sequence of a polypeptide to create an equivalent, or even an improved, variant polypeptide, one skilled in the art, for example, can change one or more of the codons of the encoding DNA sequence, e.g., according to Table 1.

TABLE 1
Amino Acid Codons

| | One | Three | |
|---|---|---|---|
| | letter | letter | |
| Amino Acids | code | code | Codons |
| Alanine | A | Ala | GCA GCC GCG GCU |
| Cysteine | C | Cys | UGC UGU |
| Aspartic acid | D | Asp | GAC GAU |
| Glutamic acid | E | Glu | GAA GAG |
| Phenylalanine | F | Phe | UUC UUU |
| Glycine | G | Gly | GGA GGC GGG GGU |
| Histidine | H | His | CAC CAU |
| Isoleucine | I | Iso | AUA AUC AUU |
| Lysine | K | Lys | AAA AAG |
| Leucine | L | Leu | UUA UUG CUA CUC CUG |
| | | | CUU |
| Methionine | M | Met | AUG |
| Asparagine | N | Asn | AAC AAU |
| Proline | P | Pro | CCA CCC CCG CCU |
| Glutamine | Q | Gln | CAA CAG |
| Arginine | R | Arg | AGA AGG CGA CGC CGG |
| | | | CGU |
| Serine | S | Ser | AGC AGU UCA UCC UCG |
| | | | UCU |
| Threonine | T | Thr | ACA ACC ACG ACU |
| Valine | V | Val | GUA GUC GUG GUU |
| Tryptophan | W | Trp | UGG |
| Tyrosine | Y | Tyr | UAC UAU |

Guidance in determining which amino acid residues can be substituted, inserted, or deleted without abolishing biological activity can be found using computer programs well known in the art, such as DNASTAR, DNA Strider, Geneious, Mac Vector, or Vector NTI software. Preferably, amino acid changes in the protein variants disclosed herein are conservative amino acid changes, i.e., substitutions of similarly charged or uncharged amino acids. A conservative amino acid change involves substitution of one of a family of amino acids which are related in their side chains. Naturally occurring amino acids are generally divided into four families: acidic (aspartate, glutamate), basic (lysine, arginine, histidine), non-polar (alanine, valine, leucine, isoleucine, proline, phenylalanine, methionine, tryptophan), and uncharged polar (glycine, asparagine, glutamine, cysteine, serine, threonine, tyrosine) amino acids. Phenylalanine, tryptophan, and tyrosine are sometimes classified jointly as aromatic amino acids. In a peptide or protein, suitable conservative substitutions of amino acids are known to those of skill in this art and generally can be made without altering a biological activity of a resulting molecule. Those of skill in this art recognize that, in general, single amino acid substitutions in non-essential regions of a polypeptide do not substantially alter biological activity (see, e.g., Watson et al. Molecular Biology of the Gene, 4th Edition, 1987, The Benjamin/Cummings Pub. Co., p. 224).

In one embodiment, where expression of two or more polypeptides is desired, the polynucleotide sequences encoding them can be separated by and IRES sequence as disclosed elsewhere herein.

Polypeptides contemplated in particular embodiments include fusion polypeptides, e.g., SEQ ID NOs: 21-23. In particular embodiments, fusion polypeptides and polynucleotides encoding fusion polypeptides are provided. Fusion polypeptides and fusion proteins refer to a polypeptide having at least two, three, four, five, six, seven, eight, nine, or ten polypeptide segments.

In another embodiment, two or more polypeptides can be expressed as a fusion protein that comprises one or more self-cleaving polypeptide sequences as disclosed elsewhere herein.

In one embodiment, a fusion protein contemplated herein comprises one or more DNA binding domains and one or more nucleases, and one or more linker and/or self-cleaving polypeptides.

In one embodiment, a fusion protein contemplated herein comprises a nuclease variant; a linker or self-cleaving peptide; and an end-processing enzyme including but not limited to a 5′-3′ exonuclease, a 5′-3′ alkaline exonuclease, and a 3′-5′ exonuclease (e.g., Trex2).

Fusion polypeptides can comprise one or more polypeptide domains or segments including, but are not limited to signal peptides, cell permeable peptide domains (CPP), DNA binding domains, nuclease domains, etc., epitope tags (e.g., maltose binding protein (“MBP”), glutathione S transferase (GST), HIS6, MYC, FLAG, V5, VSV-G, and HA), polypeptide linkers, and polypeptide cleavage signals. Fusion polypeptides are typically linked C-terminus to N-terminus, although they can also be linked C-terminus to C-terminus, N-terminus to N-terminus, or N-terminus to C-terminus. In particular embodiments, the polypeptides of the fusion protein can be in any order. Fusion polypeptides or fusion proteins can also include conservatively modified variants, polymorphic variants, alleles, mutants, subsequences, and interspecies homologs, so long as the desired activity of the fusion polypeptide is preserved. Fusion polypeptides may be produced by chemical synthetic methods or by chemical linkage between the two moieties or may generally be prepared using other standard techniques. Ligated DNA sequences comprising the fusion polypeptide are operably linked to suitable transcriptional or translational control elements as disclosed elsewhere herein.

Fusion polypeptides may optionally comprise a linker that can be used to link the one or more polypeptides or domains within a polypeptide. A peptide linker sequence may be employed to separate any two or more polypeptide components by a distance sufficient to ensure that each polypeptide folds into its appropriate secondary and tertiary structures so as to allow the polypeptide domains to exert their desired functions. Such a peptide linker sequence is incorporated into the fusion polypeptide using standard techniques in the art. Suitable peptide linker sequences may be chosen based on the following factors: (1) their ability to adopt a flexible extended conformation; (2) their inability to adopt a secondary structure that could interact with functional epitopes on the first and second polypeptides; and (3) the lack of hydrophobic or charged residues that might react with the polypeptide functional epitopes. Preferred peptide linker sequences contain Gly, Asn and Ser residues. Other near neutral amino acids, such as Thr and Ala may also be used in the linker sequence. Amino acid sequences which may be usefully employed as linkers include those disclosed in Maratea et al., Gene 40:39-46, 1985; Murphy et al., Proc. Natl. Acad. Sci. USA 83:8258-8262, 1986; U.S. Pat. Nos. 4,935,233 and 4,751,180. Linker sequences are not required when a particular fusion polypeptide segment contains non essential N-terminal amino acid regions that can be used to separate the functional domains and prevent steric interference. Preferred linkers are typically flexible amino acid subsequences which are synthesized as part of a recombinant fusion protein. Linker polypeptides can be between 1 and 200 amino acids in length, between 1 and 100 amino acids in length, or between 1 and 50 amino acids in length, including all integer values in between.

Exemplary linkers include, but are not limited to the following amino acid sequences: glycine polymers (G)ₙ; glycine-serine polymers (G1-551-5)ₙ, where n is an integer of at least one, two, three, four, or five; glycine-alanine polymers; alanine-serine polymers; GGG (SEQ ID NO: 36); DGGGS (SEQ ID NO: 37); TGEKP (SEQ ID NO: 38) (see e.g., Liu et al., PNAS 5525-5530 (1997)); GGRR (SEQ ID NO: 39) (Pomerantz et al. 1995, supra); (GGGGS)ₙ wherein n=1, 2, 3, 4 or 5 (SEQ ID NO: 40) (Kim et al., PNAS 93, 1156-1160 (1996); EGKSSGSGSESKVD (SEQ ID NO: 41) (Chaudhary et al., 1990, Proc. Natl. Acad. Sci. U.S.A. 87:1066-1070); KESGSVSSEQLAQFRSLD (SEQ ID NO: 42) (Bird et al., 1988, Science 242:423-426), GGRRGGGS (SEQ ID NO: 43) LRQRDGERP (SEQ ID NO: 44); LRQKDGGGSERP (SEQ ID NO: 45); LRQKD(GGGS)₂ERP (SEQ ID NO: 46). Alternatively, flexible linkers can be rationally designed using a computer program capable of modeling both DNA-binding sites and the peptides themselves (Desjarlais & Berg, PNAS 90:2256-2260 (1993), PNAS 91:11099-11103 (1994) or by phage display methods.

Fusion polypeptides may further comprise a polypeptide cleavage signal between each of the polypeptide domains described herein or between an endogenous open reading frame and a polypeptide encoded by a donor repair template. In addition, a polypeptide cleavage site can be put into any linker peptide sequence. Exemplary polypeptide cleavage signals include polypeptide cleavage recognition sites such as protease cleavage sites, nuclease cleavage sites (e.g., rare restriction enzyme recognition sites, self-cleaving ribozyme recognition sites), and self-cleaving viral oligopeptides (see deFelipe and Ryan, 2004. Traffic, 5(8); 616-26).

Suitable protease cleavages sites and self-cleaving peptides are known to the skilled person (see, e.g., in Ryan et al., 1997. J Gener. Virol. 78, 699-722; Scymczak et al. (2004) Nature Biotech. 5, 589-594). Exemplary protease cleavage sites include, but are not limited to the cleavage sites of potyvirus NIa proteases (e.g., tobacco etch virus protease), potyvirus HC proteases, potyvirus P1 (P35) proteases, byovirus NIa proteases, byovirus RNA-2-encoded proteases, aphthovirus L proteases, enterovirus 2A proteases, rhinovirus 2A proteases, picorna 3C proteases, comovirus 24K proteases, nepovirus 24K proteases, RTSV (rice tungro spherical virus) 3C-like protease, PYVF (parsnip yellow fleck virus) 3C-like protease, heparin, thrombin, factor Xa and enterokinase. Due to its high cleavage stringency, TEV (tobacco etch virus) protease cleavage sites are preferred in one embodiment, e.g., EXXYXQ(G/S) (SEQ ID NO: 47), for example, ENLYFQG (SEQ ID NO: 48) and ENLYFQS (SEQ ID NO: 49), wherein X represents any amino acid (cleavage by TEV occurs between Q and G or Q and S).

In certain embodiments, the self-cleaving polypeptide site comprises a 2A or 2A-like site, sequence or domain (Donnelly et al., 2001. J. Gen. Virol. 82:1027-1041). In a particular embodiment, the viral 2A peptide is an aphthovirus 2A peptide, a potyvirus 2A peptide, or a cardiovirus 2A peptide.

In one embodiment, the viral 2A peptide is selected from the group consisting of: a foot-and-mouth disease virus (FMDV) 2A peptide, an equine rhinitis A virus (ERAV) 2A peptide, a Thosea asigna virus (TaV) 2A peptide, a porcine teschovirus-1 (PTV-1) 2A peptide, a Theilovirus 2A peptide, and an encephalomyocarditis virus 2A peptide.

Illustrative examples of 2A sites are provided in Table 2.

TABLE 2
Exemplary 2A sites include the following
sequences:

| | |
|---|---|
| SEQ ID NO: 50 | GSGATNFSLLKQAGDVEENPGP |
| SEQ ID NO: 51 | ATNFSLLKQAGDVEENPGP |
| SEQ ID NO: 52 | LLKQAGDVEENPGP |
| SEQ ID NO: 53 | GSGEGRGSLLTCGDVEENPGP |
| SEQ ID NO: 54 | EGRGSLLTCGDVEENPGP |
| SEQ ID NO: 55 | LLTCGDVEENPGP |
| SEQ ID NO: 56 | GSGQCTNYALLKLAGDVESNPGP |
| SEQ ID NO: 57 | QCTNYALLKLAGDVESNPGP |
| SEQ ID NO: 58 | LLKLAGDVESNPGP |
| SEQ ID NO: 59 | GSGVKQTLNFDLLKLAGDVESNPGP |
| SEQ ID NO: 60 | VKQTLNFDLLKLAGDVESNPGP |
| SEQ ID NO: 61 | LLKLAGDVESNPGP |
| SEQ ID NO: 62 | LLNFDLLKLAGDVESNPGP |
| SEQ ID NO: 63 | TLNFDLLKLAGDVESNPGP |
| SEQ ID NO: 64 | LLKLAGDVESNPGP |
| SEQ ID NO: 65 | NFDLLKLAGDVESNPGP |
| SEQ ID NO: 66 | QLLNFDLLKLAGDVESNPGP |
| SEQ ID NO: 67 | APVKQTLNFDLLKLAGDVESNPGP |
| SEQ ID NO: 68 | VTELLYRMKRAETYCPRPLLAIHPTEARHKQKIV |
| | APVKQT |
| SEQ ID NO: 69 | LNFDLLKLAGDVESNPGP |
| SEQ ID NO: 70 | LLAIHPTEARHKQKIVAPVKQTLNFDLLKLAGDV |
| | ESNPGP |
| SEQ ID NO: 71 | EARHKQKIVAPVKQTLNFDLLKLAGDVESNPGP |

### G. Polynucleotides

In particular embodiments, polynucleotides encoding one or more homing endonuclease variants, megaTALs, end-processing enzymes, and fusion polypeptides contemplated herein are provided. As used herein, the terms “polynucleotide” or “nucleic acid” refer to deoxyribonucleic acid (DNA), ribonucleic acid (RNA) and DNA/RNA hybrids. Polynucleotides may be single-stranded or double-stranded and either recombinant, synthetic, or isolated. Polynucleotides include, but are not limited to: pre-messenger RNA (pre-mRNA), messenger RNA (mRNA), synthetic RNA, synthetic mRNA, genomic DNA (gDNA), PCR amplified DNA, complementary DNA (cDNA), synthetic DNA, and recombinant DNA. Polynucleotides refer to a polymeric form of nucleotides of at least 5, at least 10, at least 15, at least 20, at least 25, at least 30, at least 40, at least 50, at least 100, at least 200, at least 300, at least 400, at least 500, at least 1000, at least 5000, at least 10000, or at least 15000 or more nucleotides in length, either ribonucleotides or deoxyribonucleotides or a modified form of either type of nucleotide, as well as all intermediate lengths. It will be readily understood that “intermediate lengths,” in this context, means any length between the quoted values, such as 6, 7, 8, 9, etc., 101, 102, 103, etc.; 151, 152, 153, etc.; 201, 202, 203, etc. In particular embodiments, polynucleotides or variants have at least or about 50%, 55%, 60%, 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% sequence identity to a reference sequence.

In particular embodiments, polynucleotides may be codon-optimized. As used herein, the term “codon-optimized” refers to substituting codons in a polynucleotide encoding a polypeptide in order to increase the expression, stability and/or activity of the polypeptide. Factors that influence codon optimization include, but are not limited to one or more of: (i) variation of codon biases between two or more organisms or genes or synthetically constructed bias tables, (ii) variation in the degree of codon bias within an organism, gene, or set of genes, (iii) systematic variation of codons including context, (iv) variation of codons according to their decoding tRNAs, (v) variation of codons according to GC %, either overall or in one position of the triplet, (vi) variation in degree of similarity to a reference sequence for example a naturally occurring sequence, (vii) variation in the codon frequency cutoff, (viii) structural properties of mRNAs transcribed from the DNA sequence, (ix) prior knowledge about the function of the DNA sequences upon which design of the codon substitution set is to be based, and/or (x) systematic variation of codon sets for each amino acid, and/or (xi) isolated removal of spurious translation initiation sites.

As used herein the term “nucleotide” refers to a heterocyclic nitrogenous base in N-glycosidic linkage with a phosphorylated sugar. Nucleotides are understood to include natural bases, and a wide variety of art-recognized modified bases. Such bases are generally located at the 1′ position of a nucleotide sugar moiety. Nucleotides generally comprise a base, sugar and a phosphate group. In ribonucleic acid (RNA), the sugar is a ribose, and in deoxyribonucleic acid (DNA) the sugar is a deoxyribose, i.e., a sugar lacking a hydroxyl group that is present in ribose. Exemplary natural nitrogenous bases include the purines, adenosine (A) and guanidine (G), and the pyrimidines, cytidine (C) and thymidine (T) (or in the context of RNA, uracil (U)). The C-1 atom of deoxyribose is bonded to N-1 of a pyrimidine or N-9 of a purine. Nucleotides are usually mono, di- or triphosphates. The nucleotides can be unmodified or modified at the sugar, phosphate and/or base moiety, (also referred to interchangeably as nucleotide analogs, nucleotide derivatives, modified nucleotides, non-natural nucleotides, and non-standard nucleotides; see for example, WO 92/07065 and WO 93/15187). Examples of modified nucleic acid bases are summarized by Limbach et al., (1994, Nucleic Acids Res. 22, 2183-2196).

A nucleotide may also be regarded as a phosphate ester of a nucleoside, with esterification occurring on the hydroxyl group attached to C-5 of the sugar. As used herein, the term “nucleoside” refers to a heterocyclic nitrogenous base in N-glycosidic linkage with a sugar. Nucleosides are recognized in the art to include natural bases, and also to include well known modified bases. Such bases are generally located at the 1′ position of a nucleoside sugar moiety. Nucleosides generally comprise a base and sugar group. The nucleosides can be unmodified or modified at the sugar, and/or base moiety, (also referred to interchangeably as nucleoside analogs, nucleoside derivatives, modified nucleosides, non-natural nucleosides, or non-standard nucleosides). As also noted above, examples of modified nucleic acid bases are summarized by Limbach et al., (1994, Nucleic Acids Res. 22, 2183-2196).

Illustrative examples of polynucleotides include, but are not limited to polynucleotides encoding SEQ ID NOs: 1-23 and 31-32 and polynucleotide sequences set forth in SEQ ID NOs: 24-30.

In various illustrative embodiments, polynucleotides contemplated herein include, but are not limited to polynucleotides encoding homing endonuclease variants, megaTALs, end-processing enzymes, fusion polypeptides, and expression vectors, viral vectors, and transfer plasmids comprising polynucleotides contemplated herein.

As used herein, the terms “polynucleotide variant” and “variant” and the like refer to polynucleotides displaying substantial sequence identity with a reference polynucleotide sequence or polynucleotides that hybridize with a reference sequence under stringent conditions that are defined hereinafter. These terms also encompass polynucleotides that are distinguished from a reference polynucleotide by the addition, deletion, substitution, or modification of at least one nucleotide. Accordingly, the terms “polynucleotide variant” and “variant” include polynucleotides in which one or more nucleotides have been added or deleted, or modified, or replaced with different nucleotides. In this regard, it is well understood in the art that certain alterations inclusive of mutations, additions, deletions and substitutions can be made to a reference polynucleotide whereby the altered polynucleotide retains the biological function or activity of the reference polynucleotide.

In one embodiment, a polynucleotide comprises a nucleotide sequence that hybridizes to a target nucleic acid sequence under stringent conditions. To hybridize under “stringent conditions” describes hybridization protocols in which nucleotide sequences at least 60% identical to each other remain hybridized. Generally, stringent conditions are selected to be about 5° C. lower than the thermal melting point (Tm) for the specific sequence at a defined ionic strength and pH. The Tm is the temperature (under defined ionic strength, pH and nucleic acid concentration) at which 50% of the probes complementary to the target sequence hybridize to the target sequence at equilibrium. Since the target sequences are generally present at excess, at Tm, 50% of the probes are occupied at equilibrium.

The recitations “sequence identity” or, for example, comprising a “sequence 50% identical to,” as used herein, refer to the extent that sequences are identical on a nucleotide-by-nucleotide basis or an amino acid-by-amino acid basis over a window of comparison. Thus, a “percentage of sequence identity” may be calculated by comparing two optimally aligned sequences over the window of comparison, determining the number of positions at which the identical nucleic acid base (e.g., A, T, C, G, I) or the identical amino acid residue (e.g., Ala, Pro, Ser, Thr, Gly, Val, Leu, Ile, Phe, Tyr, Trp, Lys, Arg, His, Asp, Glu, Asn, Gln, Cys and Met) occurs in both sequences to yield the number of matched positions, dividing the number of matched positions by the total number of positions in the window of comparison (i.e., the window size), and multiplying the result by 100 to yield the percentage of sequence identity. Included are nucleotides and polypeptides having at least about 50%, 55%, 60%, 65%, 70%, 75%, 80%, 85%, 90%, 95%, 96%, 97%, 98%, 99% or 100% sequence identity to any of the reference sequences described herein, typically where the polypeptide variant maintains at least one biological activity of the reference polypeptide.

Terms used to describe sequence relationships between two or more polynucleotides or polypeptides include “reference sequence,” “comparison window,” “sequence identity,” “percentage of sequence identity,” and “substantial identity”. A “reference sequence” is at least 12 but frequently 15 to 18 and often at least 25 monomer units, inclusive of nucleotides and amino acid residues, in length. Because two polynucleotides may each comprise (1) a sequence (i.e., only a portion of the complete polynucleotide sequence) that is similar between the two polynucleotides, and (2) a sequence that is divergent between the two polynucleotides, sequence comparisons between two (or more) polynucleotides are typically performed by comparing sequences of the two polynucleotides over a “comparison window” to identify and compare local regions of sequence similarity. A “comparison window” refers to a conceptual segment of at least 6 contiguous positions, usually about 50 to about 100, more usually about 100 to about 150 in which a sequence is compared to a reference sequence of the same number of contiguous positions after the two sequences are optimally aligned. The comparison window may comprise additions or deletions (i.e., gaps) of about 20% or less as compared to the reference sequence (which does not comprise additions or deletions) for optimal alignment of the two sequences. Optimal alignment of sequences for aligning a comparison window may be conducted by computerized implementations of algorithms (GAP, BESTFIT, FASTA, and TFASTA in the Wisconsin Genetics Software Package Release 7.0, Genetics Computer Group, 575 Science Drive Madison, Wis., USA) or by inspection and the best alignment (i.e., resulting in the highest percentage homology over the comparison window) generated by any of the various methods selected. Reference also may be made to the BLAST family of programs as for example disclosed by Altschul et al., 1997, Nucl. Acids Res. 25:3389. A detailed discussion of sequence analysis can be found in Unit 19.3 of Ausubel et al., Current Protocols in Molecular Biology, John Wiley & Sons Inc., 1994-1998, Chapter 15.

An “isolated polynucleotide,” as used herein, refers to a polynucleotide that has been purified from the sequences which flank it in a naturally-occurring state, e.g., a DNA fragment that has been removed from the sequences that are normally adjacent to the fragment. In particular embodiments, an “isolated polynucleotide” refers to a complementary DNA (cDNA), a recombinant polynucleotide, a synthetic polynucleotide, or other polynucleotide that does not exist in nature and that has been made by the hand of man.

In various embodiments, a polynucleotide comprises an mRNA encoding a polypeptide contemplated herein including, but not limited to, a homing endonuclease variant, a megaTAL, and an end-processing enzyme. In certain embodiments, the mRNA comprises a cap, one or more nucleotides and/or modified nucleotides, and a poly(A) tail.

In particular embodiments, an mRNA contemplated herein comprises a poly(A) tail to help protect the mRNA from exonuclease degradation, stabilize the mRNA, and facilitate translation. In certain embodiments, an mRNA comprises a 3′ poly(A) tail structure.

In particular embodiments, the length of the poly(A) tail is at least about 10, 25, 50, 75, 100, 150, 200, 250, 300, 350, 400, 450, or at least about 500 or more adenine nucleotides or any intervening number of adenine nucleotides. In particular embodiments, the length of the poly(A) tail is at least about 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 202, 203, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, or 275 or more adenine nucleotides.

In particular embodiments, the length of the poly(A) tail is about 10 to about 500 adenine nucleotides, about 50 to about 500 adenine nucleotides, about 100 to about 500 adenine nucleotides, about 150 to about 500 adenine nucleotides, about 200 to about 500 adenine nucleotides, about 250 to about 500 adenine nucleotides, about 300 to about 500 adenine nucleotides, about 50 to about 450 adenine nucleotides, about 50 to about 400 adenine nucleotides, about 50 to about 350 adenine nucleotides, about 100 to about 500 adenine nucleotides, about 100 to about 450 adenine nucleotides, about 100 to about 400 adenine nucleotides, about 100 to about 350 adenine nucleotides, about 100 to about 300 adenine nucleotides, about 150 to about 500 adenine nucleotides, about 150 to about 450 adenine nucleotides, about 150 to about 400 adenine nucleotides, about 150 to about 350 adenine nucleotides, about 150 to about 300 adenine nucleotides, about 150 to about 250 adenine nucleotides, about 150 to about 200 adenine nucleotides, about 200 to about 500 adenine nucleotides, about 200 to about 450 adenine nucleotides, about 200 to about 400 adenine nucleotides, about 200 to about 350 adenine nucleotides, about 200 to about 300 adenine nucleotides, about 250 to about 500 adenine nucleotides, about 250 to about 450 adenine nucleotides, about 250 to about 400 adenine nucleotides, about 250 to about 350 adenine nucleotides, or about 250 to about 300 adenine nucleotides or any intervening range of adenine nucleotides.

Terms that describe the orientation of polynucleotides include: 5′ (normally the end of the polynucleotide having a free phosphate group) and 3′ (normally the end of the polynucleotide having a free hydroxyl (OH) group). Polynucleotide sequences can be annotated in the 5′ to 3′ orientation or the 3′ to 5′ orientation. For DNA and mRNA, the 5′ to 3′ strand is designated the “sense,” “plus,” or “coding” strand because its sequence is identical to the sequence of the pre-messenger (pre-mRNA) [except for uracil (U) in RNA, instead of thymine (T) in DNA]. For DNA and mRNA, the complementary 3′ to 5′ strand which is the strand transcribed by the RNA polymerase is designated as “template,” “antisense,” “minus,” or “non-coding” strand. As used herein, the term “reverse orientation” refers to a 5′ to 3′ sequence written in the 3′ to 5′ orientation or a 3′ to 5′ sequence written in the 5′ to 3′ orientation.

The terms “complementary” and “complementarity” refer to polynucleotides (i.e., a sequence of nucleotides) related by the base-pairing rules. For example, the complementary strand of the DNA sequence 5′ A G T C A T G 3′ is 3′ T C A G T A C 5′. The latter sequence is often written as the reverse complement with the 5′ end on the left and the 3′ end on the right, 5′ C A T G A C T 3′. A sequence that is equal to its reverse complement is said to be a palindromic sequence. Complementarity can be “partial,” in which only some of the nucleic acids' bases are matched according to the base pairing rules. Or, there can be “complete” or “total” complementarity between the nucleic acids.

The term “nucleic acid cassette” or “expression cassette” as used herein refers to genetic sequences within the vector which can express an RNA, and subsequently a polypeptide. In one embodiment, the nucleic acid cassette contains a gene(s)-of-interest, e.g., a polynucleotide(s)-of-interest. In another embodiment, the nucleic acid cassette contains one or more expression control sequences, e.g., a promoter, enhancer, poly(A) sequence, and a gene(s)-of-interest, e.g., a polynucleotide(s)-of-interest. Vectors may comprise 1, 2, 3, 4, 5, 6, 7, 8, 9 or 10 or more nucleic acid cassettes. The nucleic acid cassette is positionally and sequentially oriented within the vector such that the nucleic acid in the cassette can be transcribed into RNA, and when necessary, translated into a protein or a polypeptide, undergo appropriate post-translational modifications required for activity in the transformed cell, and be translocated to the appropriate compartment for biological activity by targeting to appropriate intracellular compartments or secretion into extracellular compartments. Preferably, the cassette has its 3′ and 5′ ends adapted for ready insertion into a vector, e.g., it has restriction endonuclease sites at each end. In a preferred embodiment, the nucleic acid cassette contains the sequence of a therapeutic gene used to treat, prevent, or ameliorate a genetic disorder. The cassette can be removed and inserted into a plasmid or viral vector as a single unit.

Polynucleotides include polynucleotide(s)-of-interest. As used herein, the term “polynucleotide-of-interest” refers to a polynucleotide encoding a polypeptide or fusion polypeptide or a polynucleotide that serves as a template for the transcription of an inhibitory polynucleotide, as contemplated herein.

Moreover, it will be appreciated by those of ordinary skill in the art that, as a result of the degeneracy of the genetic code, there are many nucleotide sequences that may encode a polypeptide, or fragment of variant thereof, as contemplated herein. Some of these polynucleotides bear minimal homology to the nucleotide sequence of any native gene. Nonetheless, polynucleotides that vary due to differences in codon usage are specifically contemplated in particular embodiments, for example polynucleotides that are optimized for human and/or primate codon selection. In one embodiment, polynucleotides comprising particular allelic sequences are provided. Alleles are endogenous polynucleotide sequences that are altered as a result of one or more mutations, such as deletions, additions and/or substitutions of nucleotides.

In a certain embodiment, a polynucleotide-of-interest comprises a donor repair template.

The polynucleotides contemplated in particular embodiments, regardless of the length of the coding sequence itself, may be combined with other DNA sequences, such as promoters and/or enhancers, untranslated regions (UTRs), Kozak sequences, polyadenylation signals, additional restriction enzyme sites, multiple cloning sites, internal ribosomal entry sites (IRES), recombinase recognition sites (e.g., LoxP, FRT, and Att sites), termination codons, transcriptional termination signals, post-transcription response elements, e.g., Woodchuck Hepatitis Virus post-transcriptional response element (WPRE), Hepatitis B Virus post-transcriptional response element (HPRE), and polynucleotides encoding self-cleaving polypeptides, epitope tags, as disclosed elsewhere herein or as known in the art, such that their overall length may vary considerably. It is therefore contemplated in particular embodiments that a polynucleotide fragment of almost any length may be employed, with the total length preferably being limited by the ease of preparation and use in the intended recombinant DNA protocol.

Polynucleotides can be prepared, manipulated, expressed and/or delivered using any of a variety of well-established techniques known and available in the art. In order to express a desired polypeptide, a nucleotide sequence encoding the polypeptide, can be inserted into appropriate vector. A desired polypeptide can also be expressed by delivering an mRNA encoding the polypeptide into the cell.

Illustrative examples of vectors include, but are not limited to plasmid, autonomously replicating sequences, and transposable elements, e.g., Sleeping Beauty, PiggyBac.

Additional illustrative examples of vectors include, without limitation, plasmids, phagemids, cosmids, artificial chromosomes such as yeast artificial chromosome (YAC), bacterial artificial chromosome (BAC), or P1-derived artificial chromosome (PAC), bacteriophages such as lambda phage or M13 phage, and animal viruses.

Illustrative examples of viruses useful as vectors include, without limitation, retrovirus (including lentivirus), adenovirus, adeno-associated virus, herpesvirus (e.g., herpes simplex virus), poxvirus, baculovirus, papillomavirus, and papovavirus (e.g., SV40).

Illustrative examples of expression vectors include, but are not limited to pClneo vectors (Promega) for expression in mammalian cells; pLenti4/V5-DEST™, pLenti6/V5-DEST™, and pLenti6.2/V5-GW/lacZ (Invitrogen) for lentivirus-mediated gene transfer and expression in mammalian cells. In particular embodiments, coding sequences of polypeptides disclosed herein can be ligated into such expression vectors for the expression of the polypeptides in mammalian cells.

In particular embodiments, the vector is an episomal vector or a vector that is maintained extrachromosomally. As used herein, the term “episomal” refers to a vector that is able to replicate without integration into host's chromosomal DNA and without gradual loss from a dividing host cell also meaning that said vector replicates extrachromosomally or episomally.

“Expression control sequences,” “control elements,” or “regulatory sequences” present in an expression vector are those non-translated regions of the vector-origin of replication, selection cassettes, promoters, enhancers, translation initiation signals (Shine Dalgarno sequence or Kozak sequence) introns, post-transcriptional regulatory elements, a polyadenylation sequence, 5′ and 3′ untranslated regions-which interact with host cellular proteins to carry out transcription and translation. Such elements may vary in their strength and specificity. Depending on the vector system and host utilized, any number of suitable transcription and translation elements, including ubiquitous promoters and inducible promoters may be used.

In particular embodiments, a polynucleotide comprises a vector, including but not limited to expression vectors and viral vectors. A vector may comprise one or more exogenous, endogenous, or heterologous control sequences such as promoters and/or enhancers. An “endogenous control sequence” is one which is naturally linked with a given gene in the genome. An “exogenous control sequence” is one which is placed in juxtaposition to a gene by means of genetic manipulation (i.e., molecular biological techniques) such that transcription of that gene is directed by the linked enhancer/promoter. A “heterologous control sequence” is an exogenous sequence that is from a different species than the cell being genetically manipulated. A “synthetic” control sequence may comprise elements of one more endogenous and/or exogenous sequences, and/or sequences determined in vitro or in silico that provide optimal promoter and/or enhancer activity for the particular therapy.

The term “promoter” as used herein refers to a recognition site of a polynucleotide (DNA or RNA) to which an RNA polymerase binds. An RNA polymerase initiates and transcribes polynucleotides operably linked to the promoter. In particular embodiments, promoters operative in mammalian cells comprise an AT-rich region located approximately 25 to 30 bases upstream from the site where transcription is initiated and/or another sequence found 70 to 80 bases upstream from the start of transcription, a CNCAAT region where N may be any nucleotide.

The term “enhancer” refers to a segment of DNA which contains sequences capable of providing enhanced transcription and in some instances can function independent of their orientation relative to another control sequence. An enhancer can function cooperatively or additively with promoters and/or other enhancer elements. The term “promoter/enhancer” refers to a segment of DNA which contains sequences capable of providing both promoter and enhancer functions.

The term “operably linked”, refers to a juxtaposition wherein the components described are in a relationship permitting them to function in their intended manner. In one embodiment, the term refers to a functional linkage between a nucleic acid expression control sequence (such as a promoter, and/or enhancer) and a second polynucleotide sequence, e.g., a polynucleotide-of-interest, wherein the expression control sequence directs transcription of the nucleic acid corresponding to the second sequence.

As used herein, the term “constitutive expression control sequence” refers to a promoter, enhancer, or promoter/enhancer that continually or continuously allows for transcription of an operably linked sequence. A constitutive expression control sequence may be a “ubiquitous” promoter, enhancer, or promoter/enhancer that allows expression in a wide variety of cell and tissue types or a “cell specific,” “cell type specific,” “cell lineage specific,” or “tissue specific” promoter, enhancer, or promoter/enhancer that allows expression in a restricted variety of cell and tissue types, respectively.

Illustrative ubiquitous expression control sequences suitable for use in particular embodiments include, but are not limited to, a cytomegalovirus (CMV) immediate early promoter, a viral simian virus 40 (SV40) (e.g., early or late), a Moloney murine leukemia virus (MoMLV) LTR promoter, a Rous sarcoma virus (RSV) LTR, a herpes simplex virus (HSV) (thymidine kinase) promoter, H5, P7.5, and P11 promoters from vaccinia virus, a short elongation factor 1-alpha (EF1a-short) promoter, a long elongation factor 1-alpha (EF1a-long) promoter, early growth response 1 (EGR1), ferritin H (FerH), ferritin L (FerL), Glyceraldehyde 3-phosphate dehydrogenase (GAPDH), eukaryotic translation initiation factor 4A1 (EIF4A1), heat shock 70 kDa protein 5 (HSPAS), heat shock protein 90 kDa beta, member 1 (HSP90B1), heat shock protein 70 kDa (HSP70), β-kinesin ((3-KIN), the human ROSA 26 locus (Irions et al., Nature Biotechnology 25, 1477-1482 (2007)), a Ubiquitin C promoter (UBC), a phosphoglycerate kinase-1 (PGK) promoter, a cytomegalovirus enhancer/chicken β-actin (CAG) promoter, a β-actin promoter and a myeloproliferative sarcoma virus enhancer, negative control region deleted, d1587rev primer-binding site substituted (MND) promoter (Challita et al., J Virol. 69(2):748-55 (1995)).

In a particular embodiment, it may be desirable to use a cell, cell type, cell lineage or tissue specific expression control sequence to achieve cell type specific, lineage specific, or tissue specific expression of a desired polynucleotide sequence (e.g., to express a particular nucleic acid encoding a polypeptide in only a subset of cell types, cell lineages, or tissues or during specific stages of development).

As used herein, “conditional expression” may refer to any type of conditional expression including, but not limited to, inducible expression; repressible expression; expression in cells or tissues having a particular physiological, biological, or disease state, etc. This definition is not intended to exclude cell type or tissue specific expression. Certain embodiments provide conditional expression of a polynucleotide-of-interest, e.g., expression is controlled by subjecting a cell, tissue, organism, etc., to a treatment or condition that causes the polynucleotide to be expressed or that causes an increase or decrease in expression of the polynucleotide encoded by the polynucleotide-of-interest.

Illustrative examples of inducible promoters/systems include, but are not limited to, steroid-inducible promoters such as promoters for genes encoding glucocorticoid or estrogen receptors (inducible by treatment with the corresponding hormone), metallothionine promoter (inducible by treatment with various heavy metals), MX-1 promoter (inducible by interferon), the “GeneSwitch” mifepristone-regulatable system (Sirin et al., 2003, Gene, 323:67), the cumate inducible gene switch (WO 2002/088346), tetracycline-dependent regulatory systems, etc.

Conditional expression can also be achieved by using a site-specific DNA recombinase. According to certain embodiments, polynucleotides comprise at least one (typically two) site(s) for recombination mediated by a site-specific recombinase. As used herein, the terms “recombinase” or “site-specific recombinase” include excisive or integrative proteins, enzymes, co-factors or associated proteins that are involved in recombination reactions involving one or more recombination sites (e.g., two, three, four, five, six, seven, eight, nine, ten or more), which may be wild-type proteins (see Landy, Current Opinion in Biotechnology 3:699-707 (1993)), or mutants, derivatives (e.g., fusion proteins containing the recombination protein sequences or fragments thereof), fragments, and variants thereof. Illustrative examples of recombinases suitable for use in particular embodiments include, but are not limited to: Cre, Int, IHF, Xis, Flp, Fis, Hin, Gin, ΦC31, CM, Tn3 resolvase, TndX, XerC, XerD, TnpX, Hjc, Gin, SpCCE1, and ParA.

The polynucleotides may comprise one or more recombination sites for any of a wide variety of site-specific recombinases. It is to be understood that the target site for a site-specific recombinase is in addition to any site(s) required for integration of a vector, e.g., a retroviral vector or lentiviral vector. As used herein, the terms “recombination sequence,” “recombination site,” or “site-specific recombination site” refer to a particular nucleic acid sequence to which a recombinase recognizes and binds.

In particular embodiments, polynucleotides contemplated herein, include one or more polynucleotides-of-interest that encode one or more polypeptides. In particular embodiments, to achieve efficient translation of each of the plurality of polypeptides, the polynucleotide sequences can be separated by one or more IRES sequences or polynucleotide sequences encoding self-cleaving polypeptides.

As used herein, an “internal ribosome entry site” or “IRES” refers to an element that promotes direct internal ribosome entry to the initiation codon, such as ATG, of a cistron (a protein encoding region), thereby leading to the cap-independent translation of the gene. See, e.g., Jackson et al., 1990. Trends Biochem Sci 15(12):477-83) and Jackson and Kaminski. 1995. RNA 1(10):985-1000. Examples of IRES generally employed by those of skill in the art include those described in U.S. Pat. No. 6,692,736. Further examples of “IRES” known in the art include, but are not limited to IRES obtainable from picornavirus (Jackson et al., 1990) and IRES obtainable from viral or cellular mRNA sources, such as for example, immunoglobulin heavy-chain binding protein (BiP), the vascular endothelial growth factor (VEGF) (Huez et al. 1998. Mol. Cell. Biol. 18(11):6178-6190), the fibroblast growth factor 2 (FGF-2), and insulin-like growth factor (IGFII), the translational initiation factor eIF4G and yeast transcription factors TFIID and HAP4, the encephelomycarditis virus (EMCV) which is commercially available from Novagen (Duke et al., 1992. J. Virol 66(3):1602-9) and the VEGF IRES (Huez et al., 1998. Mol Cell Biol 18(11):6178-90). IRES have also been reported in viral genomes of Picornaviridae, Dicistroviridae and Flaviviridae species and in HCV, Friend murine leukemia virus (FrMLV) and Moloney murine leukemia virus (MoMLV).

In particular embodiments, the polynucleotides comprise polynucleotides that have a consensus Kozak sequence and that encode a desired polypeptide. As used herein, the term “Kozak sequence” refers to a short nucleotide sequence that greatly facilitates the initial binding of mRNA to the small subunit of the ribosome and increases translation. The consensus Kozak sequence is (GCC)RCCATGG (SEQ ID NO:72), where R is a purine (A or G) (Kozak, 1986. Cell. 44(2):283-92, and Kozak, 1987. Nucleic Acids Res. 15(20):8125-48).

Elements directing the efficient termination and polyadenylation of the heterologous nucleic acid transcripts increases heterologous gene expression. Transcription termination signals are generally found downstream of the polyadenylation signal. In particular embodiments, vectors comprise a polyadenylation sequence 3′ of a polynucleotide encoding a polypeptide to be expressed. The term “polyA site” or “polyA sequence” as used herein denotes a DNA sequence which directs both the termination and polyadenylation of the nascent RNA transcript by RNA polymerase II. Polyadenylation sequences can promote mRNA stability by addition of a polyA tail to the 3′ end of the coding sequence and thus, contribute to increased translational efficiency. Cleavage and polyadenylation is directed by a poly(A) sequence in the RNA. The core poly(A) sequence for mammalian pre-mRNAs has two recognition elements flanking a cleavage-polyadenylation site. Typically, an almost invariant AAUAAA hexamer lies 20-50 nucleotides upstream of a more variable element rich in U or GU residues. Cleavage of the nascent transcript occurs between these two elements and is coupled to the addition of up to 250 adenosines to the 5′ cleavage product. In particular embodiments, the core poly(A) sequence is an ideal polyA sequence (e.g., AATAAA, ATTAAA, AGTAAA). In particular embodiments, the poly(A) sequence is an SV40 polyA sequence, a bovine growth hormone polyA sequence (BGHpA), a rabbit β-globin polyA sequence (rβgpA), variants thereof, or another suitable heterologous or endogenous polyA sequence known in the art.

In particular embodiments, polynucleotides encoding one or more homing endonuclease variants, megaTALs, end-processing enzymes, or fusion polypeptides may be introduced into hematopoietic cells, e.g., CD34⁺ cells, by both non-viral and viral methods. In particular embodiments, delivery of one or more polynucleotides encoding nucleases and/or donor repair templates may be provided by the same method or by different methods, and/or by the same vector or by different vectors.

The term “vector” is used herein to refer to a nucleic acid molecule capable transferring or transporting another nucleic acid molecule. The transferred nucleic acid is generally linked to, e.g., inserted into, the vector nucleic acid molecule. A vector may include sequences that direct autonomous replication in a cell, or may include sequences sufficient to allow integration into host cell DNA. In particular embodiments, non-viral vectors are used to deliver one or more polynucleotides contemplated herein to a CD34+ cell.

Illustrative examples of non-viral vectors include, but are not limited to plasmids (e.g., DNA plasmids or RNA plasmids), transposons, cosmids, and bacterial artificial chromosomes.

Illustrative methods of non-viral delivery of polynucleotides contemplated in particular embodiments include, but are not limited to: electroporation, sonoporation, lipofection, microinjection, biolistics, virosomes, liposomes, immunoliposomes, nanoparticles, polycation or lipid:nucleic acid conjugates, naked DNA, artificial virions, DEAE-dextran-mediated transfer, gene gun, and heat-shock.

Illustrative examples of polynucleotide delivery systems suitable for use in particular embodiments contemplated in particular embodiments include, but are not limited to those provided by Amaxa Biosystems, Maxcyte, Inc., BTX Molecular Delivery Systems, and Copernicus Therapeutics Inc. Lipofection reagents are sold commercially (e.g., Transfectam™ and Lipofectin™). Cationic and neutral lipids that are suitable for efficient receptor-recognition lipofection of polynucleotides have been described in the literature. See e.g., Liu et al. (2003) Gene Therapy. 10:180-187; and Balazs et al. (2011) Journal of Drug Delivery. 2011:1-12. Antibody-targeted, bacterially derived, non-living nanocell-based delivery is also contemplated in particular embodiments.

Viral vectors comprising polynucleotides contemplated in particular embodiments can be delivered in vivo by administration to an individual patient, typically by systemic administration (e.g., intravenous, intraperitoneal, intramuscular, subdermal, or intracranial infusion) or topical application, as described below. Alternatively, vectors can be delivered to cells ex vivo, such as cells explanted from an individual patient (e.g., mobilized peripheral blood, lymphocytes, bone marrow aspirates, tissue biopsy, etc.) or universal donor hematopoietic stem cells, followed by reimplantation of the cells into a patient.

In one embodiment, viral vectors comprising nuclease variants and/or donor repair templates are administered directly to an organism for transduction of cells in vivo. Alternatively, naked DNA or mRNA can be administered. Administration is by any of the routes normally used for introducing a molecule into ultimate contact with blood or tissue cells including, but not limited to, injection, infusion, topical application and electroporation. Suitable methods of administering such nucleic acids are available and well known to those of skill in the art, and, although more than one route can be used to administer a particular composition, a particular route can often provide a more immediate and more effective reaction than another route.

Illustrative examples of viral vector systems suitable for use in particular embodiments contemplated herein include, but are not limited to adeno-associated virus (AAV), retrovirus, herpes simplex virus, adenovirus, and vaccinia virus vectors.

### H. Genome Edited Cells

The genome edited cells manufactured by the methods contemplated in particular embodiments provide improved cell-based therapeutics for the treatment of X-linked agammaglobulinemia (XLA). Without wishing to be bound to any particular theory, it is believed that the compositions and methods contemplated herein can be used to introduce a polynucleotide encoding a functional BTK polypeptide into a BTK gene that comprises one or more mutations and/or deletions that result in little or no endogenous BTK expression and XLA; and thus, provide a more robust genome edited cell composition that may be used to treat, and in some embodiments potentially cure, XLA.

Genome edited cells contemplated in particular embodiments may be autologous/autogeneic (“self”) or non-autologous (“non-self,” e.g., allogeneic, syngeneic or xenogeneic). “Autologous,” as used herein, refers to cells from the same subject. “Allogeneic,” as used herein, refers to cells of the same species that differ genetically to the cell in comparison. “Syngeneic,” as used herein, refers to cells of a different subject that are genetically identical to the cell in comparison. “Xenogeneic,” as used herein, refers to cells of a different species to the cell in comparison. In preferred embodiments, the cells are obtained from a mammalian subject. In a more preferred embodiment, the cells are obtained from a primate subject, optionally a non-human primate. In the most preferred embodiment, the cells are obtained from a human subject.

An “isolated cell” refers to a non-naturally occurring cell, e.g., a cell that does not exist in nature, a modified cell, an engineered cell, etc., that has been obtained from an in vivo tissue or organ and is substantially free of extracellular matrix.

Illustrative examples of cell types whose genome can be edited using the compositions and methods contemplated herein include, but are not limited to, cell lines, primary cells, stem cells, progenitor cells, and differentiated cells.

The term “stem cell” refers to a cell which is an undifferentiated cell capable of (1) long term self-renewal, or the ability to generate at least one identical copy of the original cell, (2) differentiation at the single cell level into multiple, and in some instance only one, specialized cell type and (3) of in vivo functional regeneration of tissues. Stem cells are subclassified according to their developmental potential as totipotent, pluripotent, multipotent and oligo/unipotent. “Self-renewal” refers a cell with a unique capacity to produce unaltered daughter cells and to generate specialized cell types (potency). Self-renewal can be achieved in two ways. Asymmetric cell division produces one daughter cell that is identical to the parental cell and one daughter cell that is different from the parental cell and is a progenitor or differentiated cell. Symmetric cell division produces two identical daughter cells. “Proliferation” or “expansion” of cells refers to symmetrically dividing cells.

As used herein, the term “progenitor” or “progenitor cells” refers to cells have the capacity to self-renew and to differentiate into more mature cells. Many progenitor cells differentiate along a single lineage, but may have quite extensive proliferative capacity.

In particular embodiments, the cell is a primary cell. The term “primary cell” as used herein is known in the art to refer to a cell that has been isolated from a tissue and has been established for growth in vitro or ex vivo. Corresponding cells have undergone very few, if any, population doublings and are therefore more representative of the main functional component of the tissue from which they are derived in comparison to continuous cell lines, thus representing a more representative model to the in vivo state. Methods to obtain samples from various tissues and methods to establish primary cell lines are well-known in the art (see, e.g., Jones and Wise, Methods Mol Biol. 1997). Primary cells for use in the methods contemplated herein are derived from umbilical cord blood, placental blood, mobilized peripheral blood and bone marrow. In one embodiment, the primary cell is a hematopoietic stem or progenitor cell.

In one embodiment, the genome edited cell is an embryonic stem cell.

In one embodiment, the genome edited cell is an adult stem or progenitor cell.

In one embodiment, the genome edited cell is primary cell.

In a preferred embodiment, the genome edited cell is a hematopoietic cell, e.g., hematopoietic stem cell, hematopoietic progenitor cell, such as a B cell progenitor cell, or cell population comprising hematopoietic cells.

As used herein, the term “population of cells” refers to a plurality of cells that may be made up of any number and/or combination of homogenous or heterogeneous cell types, as described elsewhere herein. For example, for transduction of hematopoietic stem or progenitor cells, a population of cells may be isolated or obtained from umbilical cord blood, placental blood, bone marrow, or mobilized peripheral blood. A population of cells may comprise about 10%, about 20%, about 30%, about 40%, about 50%, about 60%, about 70%, about 80%, about 90%, or about 100% of the target cell type to be edited. In certain embodiments, hematopoietic stem or progenitor cells may be isolated or purified from a population of heterogeneous cells using methods known in the art.

Illustrative sources to obtain hematopoietic cells include, but are not limited to: cord blood, bone marrow or mobilized peripheral blood.

Hematopoietic stem cells (HSCs) give rise to committed hematopoietic progenitor cells (HPCs) that are capable of generating the entire repertoire of mature blood cells over the lifetime of an organism. The term “hematopoietic stem cell” or “HSC” refers to multipotent stem cells that give rise to the all the blood cell types of an organism, including myeloid (e.g., monocytes and macrophages, neutrophils, basophils, eosinophils, erythrocytes, megakaryocytes/platelets, dendritic cells), and lymphoid lineages (e.g., T-cells, B-cells, NK-cells), and others known in the art (See Fei, R., et al., U.S. Pat. No. 5,635,387; McGlave, et al., U.S. Pat. No. 5,460,964; Simmons, P., et al., U.S. Pat. No. 5,677,136; Tsukamoto, et al., U.S. Pat. No. 5,750,397; Schwartz, et al., U.S. Pat. No. 5,759,793; DiGuisto, et al., U.S. Pat. No. 5,681,599; Tsukamoto, et al., U.S. Pat. No. 5,716,827). When transplanted into lethally irradiated animals or humans, hematopoietic stem and progenitor cells can repopulate the erythroid, neutrophil-macrophage, megakaryocyte and lymphoid hematopoietic cell pool.

Additional illustrative examples of hematopoietic stem or progenitor cells suitable for use with the methods and compositions contemplated herein include hematopoietic cells that are CD34⁺CD38^{Lo}CD90⁺CD45^{RA-}, hematopoietic cells that are CD34⁺, CD59⁺, Thy1/CD90⁺, CD38^{Lo/-}, C-kit/CD117⁺, and Line), and hematopoietic cells that are CD133⁺.

In a preferred embodiment, the hematopoietic cells that are CD133⁺CD90⁺.

In a preferred embodiment, the hematopoietic cells that are CD133⁺CD34⁺.

In a preferred embodiment, the hematopoietic cells that are CD133⁺CD90⁺CD34⁺.

Various methods exist to characterize hematopoietic hierarchy. One method of characterization is the SLAM code. The SLAM (Signaling lymphocyte activation molecule) family is a group of >10 molecules whose genes are located mostly tandemly in a single locus on chromosome 1 (mouse), all belonging to a subset of immunoglobulin gene superfamily, and originally thought to be involved in T-cell stimulation. This family includes CD48, CD150, CD244, etc., CD150 being the founding member, and, thus, also called slamF1, i.e., SLAM family member 1. The signature SLAM code for the hematopoietic hierarchy is hematopoietic stem cells (HSC)—CD150⁺CD48⁻CD244⁻; multipotent progenitor cells (MPPs)—CD150⁻CD48⁻CD244⁺; lineage-restricted progenitor cells (LRPs)—CD150⁻CD48⁺CD244⁺; common myeloid progenitor (CMP)—lin-SCA-1-c-kit⁺CD34⁺CD16/32^{mid}; granulocyte-macrophage progenitor (GMP)—lin⁻SCA-1-c-kit⁺CD34⁺CD16/32^{hi}; and megakaryocyte-erythroid progenitor (MEP)—lin⁻SCA-1-c-kit⁺CD34⁻CD16/32^{low}.

Preferred target cell types edited with the compositions and methods contemplated herein include, hematopoietic cells, preferably human hematopoietic cells, more preferably human hematopoietic stem and progenitor cells, and even more preferably CD34+ human hematopoietic stem cells. The term “CD34+ cell,” as used herein refers to a cell expressing the CD34 protein on its cell surface. “CD34,” as used herein refers to a cell surface glycoprotein (e.g., sialomucin protein) that often acts as a cell-cell adhesion factor. CD34+ is a cell surface marker of both hematopoietic stem and progenitor cells.

In one embodiment, the genome edited hematopoietic cells are CD150⁺CD48⁻CD244⁻ cells.

In one embodiment, the genome edited hematopoietic cells are CD34⁺CD133⁺ cells.

In one embodiment, the genome edited hematopoietic cells are CD133⁺ cells.

In one embodiment, the genome edited hematopoietic cells are CD34⁺ cells.

In particular embodiments, a population of hematopoietic cells comprising hematopoietic stem and progenitor cells (HSPCs) comprises a defective BTK gene edited to express a functional BTK polypeptide, wherein the edit is a DSB repaired by HDR.

In particular embodiments, the genome edited cells comprise B cell progenitor cells.

In particular embodiments, the genome edited cells comprise one or more mutations and/or deletions in a BTK gene that result in little or no endogenous BTK expression.

### I. Compositions and Formulations

The compositions contemplated in particular embodiments may comprise one or more polypeptides, polynucleotides, vectors comprising same, and genome editing compositions and genome edited cell compositions, as contemplated herein. The genome editing compositions and methods contemplated in particular embodiments are useful for editing a target site in the human BTK gene in a cell or a population of cells. In preferred embodiments, a genome editing composition is used to edit a BTK gene by HDR in a hematopoietic cell, e.g., a hematopoietic stem or progenitor cell, or a CD34⁺ cell.

In various embodiments, the compositions contemplated herein comprise a nuclease variant, and optionally an end-processing enzyme, e.g., a 3′-5′ exonuclease (Trex2). The nuclease variant may be in the form of an mRNA that is introduced into a cell via polynucleotide delivery methods disclosed supra, e.g., electroporation, lipid nanoparticles, etc. In one embodiment, a composition comprising an mRNA encoding a homing endonuclease variant or megaTAL, and optionally a 3′-5′ exonuclease, is introduced in a cell via polynucleotide delivery methods disclosed supra.

In particular embodiments, the compositions contemplated herein comprise a population of cells, a nuclease variant, and optionally, a donor repair template. In particular embodiments, the compositions contemplated herein comprise a population of cells, a nuclease variant, an end-processing enzyme, and optionally, a donor repair template. The nuclease variant and/or end-processing enzyme may be in the form of an mRNA that is introduced into the cell via polynucleotide delivery methods disclosed supra. The donor repair template may also be introduced into the cell by means of a separate composition.

In particular embodiments, the compositions contemplated herein comprise a population of cells, a homing endonuclease variant or megaTAL, and optionally, a donor repair template. In particular embodiments, the compositions contemplated herein comprise a population of cells, a homing endonuclease variant or megaTAL, a 3′-5′ exonuclease, and optionally, a donor repair template. The homing endonuclease variant, megaTAL, and/or 3′-5′ exonuclease may be in the form of an mRNA that is introduced into the cell via polynucleotide delivery methods disclosed supra. The donor repair template may also be introduced into the cell by means of a separate composition.

In particular embodiments, the population of cells comprise genetically modified hematopoietic cells including, but not limited to, hematopoietic stem cells, hematopoietic progenitor cells, CD133⁺ cells, and CD34⁺ cells.

Compositions include, but are not limited to pharmaceutical compositions. A “pharmaceutical composition” refers to a composition formulated in pharmaceutically-acceptable or physiologically-acceptable solutions for administration to a cell or an animal, either alone, or in combination with one or more other modalities of therapy. It will also be understood that, if desired, the compositions may be administered in combination with other agents as well, such as, e.g., cytokines, growth factors, hormones, small molecules, chemotherapeutics, pro-drugs, drugs, antibodies, or other various pharmaceutically-active agents. There is virtually no limit to other components that may also be included in the compositions, provided that the additional agents do not adversely affect the composition.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The term “pharmaceutically acceptable carrier” refers to a diluent, adjuvant, excipient, or vehicle with which the therapeutic cells are administered. Illustrative examples of pharmaceutical carriers can be sterile liquids, such as cell culture media, water and oils, including those of petroleum, animal, vegetable or synthetic origin, such as peanut oil, soybean oil, mineral oil, sesame oil and the like. Saline solutions and aqueous dextrose and glycerol solutions can also be employed as liquid carriers, particularly for injectable solutions. Suitable pharmaceutical excipients in particular embodiments, include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. Except insofar as any conventional media or agent is incompatible with the active ingredient, its use in the therapeutic compositions is contemplated. Supplementary active ingredients can also be incorporated into the compositions.

In one embodiment, a composition comprising a pharmaceutically acceptable carrier is suitable for administration to a subject. In particular embodiments, a composition comprising a carrier is suitable for parenteral administration, e.g., intravascular (intravenous or intraarterial), intraperitoneal or intramuscular administration. In particular embodiments, a composition comprising a pharmaceutically acceptable carrier is suitable for intraventricular, intraspinal, or intrathecal administration. Pharmaceutically acceptable carriers include sterile aqueous solutions, cell culture media, or dispersions. The use of such media and agents for pharmaceutically active substances is well known in the art. Except insofar as any conventional media or agent is incompatible with the transduced cells, use thereof in the pharmaceutical compositions is contemplated.

In particular embodiments, compositions contemplated herein comprise genetically modified hematopoietic stem and/or progenitor cells comprising an exogenous polynucleotide encoding a functional BTK polypeptide and a pharmaceutically acceptable carrier.

In particular embodiments, compositions contemplated herein comprise genetically modified hematopoietic stem and/or progenitor cells comprising a BTK gene comprising one or more mutations and/or deletions and an exogenous polynucleotide encoding a functional BTK polypeptide and a pharmaceutically acceptable carrier. A composition comprising a cell-based composition contemplated herein can be administered by parenteral administration methods.

The pharmaceutically acceptable carrier must be of sufficiently high purity and of sufficiently low toxicity to render it suitable for administration to the human subject being treated. It further should maintain or increase the stability of the composition. The pharmaceutically acceptable carrier can be liquid or solid and is selected, with the planned manner of administration in mind, to provide for the desired bulk, consistency, etc., when combined with other components of the composition. For example, the pharmaceutically acceptable carrier can be, without limitation, a binding agent (e.g., pregelatinized maize starch, polyvinylpyrrolidone or hydroxypropyl methylcellulose, etc.), a filler (e.g., lactose and other sugars, microcrystalline cellulose, pectin, gelatin, calcium sulfate, ethyl cellulose, polyacrylates, calcium hydrogen phosphate, etc.), a lubricant (e.g., magnesium stearate, talc, silica, colloidal silicon dioxide, stearic acid, metallic stearates, hydrogenated vegetable oils, corn starch, polyethylene glycols, sodium benzoate, sodium acetate, etc.), a disintegrant (e.g., starch, sodium starch glycolate, etc.), or a wetting agent (e.g., sodium lauryl sulfate, etc.). Other suitable pharmaceutically acceptable carriers for the compositions contemplated herein include, but are not limited to, water, salt solutions, alcohols, polyethylene glycols, gelatins, amyloses, magnesium stearates, talcs, silicic acids, viscous paraffins, hydroxymethylcelluloses, polyvinylpyrrolidones and the like.

Such carrier solutions also can contain buffers, diluents and other suitable additives. The term “buffer” as used herein refers to a solution or liquid whose chemical makeup neutralizes acids or bases without a significant change in pH. Examples of buffers contemplated herein include, but are not limited to, Dulbecco's phosphate buffered saline (PBS), Ringer's solution, 5% dextrose in water (D5W), normal/physiologic saline (0.9% NaCl).

The pharmaceutically acceptable carriers may be present in amounts sufficient to maintain a pH of the composition of about 7. Alternatively, the composition has a pH in a range from about 6.8 to about 7.4, e.g., 6.8, 6.9, 7.0, 7.1, 7.2, 7.3, and 7.4. In still another embodiment, the composition has a pH of about 7.4.

Compositions contemplated herein may comprise a nontoxic pharmaceutically acceptable medium. The compositions may be a suspension. The term “suspension” as used herein refers to non-adherent conditions in which cells are not attached to a solid support. For example, cells maintained as a suspension may be stirred or agitated and are not adhered to a support, such as a culture dish.

In particular embodiments, compositions contemplated herein are formulated in a suspension, where the genome edited hematopoietic stem and/or progenitor cells are dispersed within an acceptable liquid medium or solution, e.g., saline or serum-free medium, in an intravenous (IV) bag or the like. Acceptable diluents include, but are not limited to water, PlasmaLyte, Ringer's solution, isotonic sodium chloride (saline) solution, serum-free cell culture medium, and medium suitable for cryogenic storage, e.g., Cryostor® medium.

In certain embodiments, a pharmaceutically acceptable carrier is substantially free of natural proteins of human or animal origin, and suitable for storing a composition comprising a population of genome edited cells, e.g., hematopoietic stem and progenitor cells. The therapeutic composition is intended to be administered into a human patient, and thus is substantially free of cell culture components such as bovine serum albumin, horse serum, and fetal bovine serum.

In some embodiments, compositions are formulated in a pharmaceutically acceptable cell culture medium. Such compositions are suitable for administration to human subjects. In particular embodiments, the pharmaceutically acceptable cell culture medium is a serum free medium.

Serum-free medium has several advantages over serum containing medium, including a simplified and better defined composition, a reduced degree of contaminants, elimination of a potential source of infectious agents, and lower cost. In various embodiments, the serum-free medium is animal-free, and may optionally be protein-free. Optionally, the medium may contain biopharmaceutically acceptable recombinant proteins. “Animal-free” medium refers to medium wherein the components are derived from non-animal sources. Recombinant proteins replace native animal proteins in animal-free medium and the nutrients are obtained from synthetic, plant or microbial sources. “Protein-free” medium, in contrast, is defined as substantially free of protein.

Illustrative examples of serum-free media used in particular compositions include, but are not limited to QBSF-60 (Quality Biological, Inc.), StemPro-34 (Life Technologies), and X-VIVO 10.

In a preferred embodiment, the compositions comprising genome edited hematopoietic stem and/or progenitor cells are formulated in PlasmaLyte.

In various embodiments, compositions comprising hematopoietic stem and/or progenitor cells are formulated in a cryopreservation medium. For example, cryopreservation media with cryopreservation agents may be used to maintain a high cell viability outcome post-thaw. Illustrative examples of cryopreservation media used in particular compositions include, but are not limited to, CryoStor CS10, CryoStor CS5, and CryoStor CS2.

In one embodiment, the compositions are formulated in a solution comprising 50:50 PlasmaLyte A to CryoStor CS10.

In particular embodiments, the composition is substantially free of mycoplasma, endotoxin, and microbial contamination. By “substantially free” with respect to endotoxin is meant that there is less endotoxin per dose of cells than is allowed by the FDA for a biologic, which is a total endotoxin of 5 EU/kg body weight per day, which for an average 70 kg person is 350 EU per total dose of cells. In particular embodiments, compositions comprising hematopoietic stem or progenitor cells transduced with a retroviral vector contemplated herein contains about 0.5 EU/mL to about 5.0 EU/mL, or about 0.5 EU/mL, 1.0 EU/mL, 1.5 EU/mL, 2.0 EU/mL, 2.5 EU/mL, 3.0 EU/mL, 3.5 EU/mL, 4.0 EU/mL, 4.5 EU/mL, or 5.0 EU/mL.

In certain embodiments, compositions and formulations suitable for the delivery of polynucleotides are contemplated including, but not limited to, one or more mRNAs encoding one or more reprogrammed nucleases, and optionally end-processing enzymes.

Exemplary formulations for ex vivo delivery may also include the use of various transfection agents known in the art, such as calcium phosphate, electroporation, heat shock and various liposome formulations (i.e., lipid-mediated transfection). Liposomes, as described in greater detail below, are lipid bilayers entrapping a fraction of aqueous fluid. DNA spontaneously associates to the external surface of cationic liposomes (by virtue of its charge) and these liposomes will interact with the cell membrane.

In particular embodiments, formulation of pharmaceutically-acceptable carrier solutions is well-known to those of skill in the art, as is the development of suitable dosing and treatment regimens for using the particular compositions described herein in a variety of treatment regimens, including e.g., enteral and parenteral, e.g., intravascular, intravenous, intraarterial, intraosseously, intraventricular, intracerebral, intracranial, intraspinal, intrathecal, and intramedullary administration and formulation. It would be understood by the skilled artisan that particular embodiments contemplated herein may comprise other formulations, such as those that are well known in the pharmaceutical art, and are described, for example, in Remington: The Science and Practice of Pharmacy, volume I and volume II. 22^{nd} Edition. Edited by Loyd V. Allen Jr. Philadelphia, Pa.: Pharmaceutical Press; 2012, which is incorporated by reference herein, in its entirety.

### J. Genome Edited Cell Therapies

The genome edited cells manufactured by the methods contemplated in particular embodiments provide improved drug products for use in the prevention, treatment, and amelioration of X-linked agammaglobulinemia (XLA) or for preventing, treating, or ameliorating at least one symptom associated with XLA or a subject having an XLA causing mutation in a BTK gene. As used herein, the term “drug product” refers to genetically modified cells produced using the compositions and methods contemplated herein. In particular embodiments, the drug product comprises genetically modified hematopoietic stem or progenitor cells, e.g., CD34⁺ cells. The genetically modified hematopoietic stem or progenitor cells give rise to the entire B cell lineage, whereas non-modified cells comprising one or more mutations and/or deletions in a BTK gene that lead to XLA are defective in B cell development.

In particular embodiments, hematopoietic stem or progenitor cells that will be edited comprise a non-functional or disrupted, ablated, or partially deleted BTK gene, thereby reducing or eliminating BTK expression and abrogating normal B cell development.

In particular embodiments, genome edited hematopoietic stem or progenitor cells comprise a non-functional or disrupted, ablated, or partially deleted BTK gene, thereby reducing or eliminating endogenous BTK expression and further comprise a polynucleotide, inserted into the BTK gene, encoding a functional BTK polypeptide that restores normal B cell development.

In particular embodiments, genome edited hematopoietic stem or progenitor cells provide a curative, preventative, or ameliorative therapy to a subject diagnosed with or that is suspected of having XLA.

In various embodiments, the genome editing compositions are administered by direct injection to a cell, tissue, or organ of a subject in need of gene therapy, in vivo, e.g., bone marrow. In various other embodiments, cells are edited in vitro or ex vivo with reprogrammed nucleases contemplated herein, and optionally expanded ex vivo. The genome edited cells are then administered to a subject in need of therapy.

Preferred cells for use in the genome editing methods contemplated herein include autologous/autogeneic (“self”) cells, preferably hematopoietic cells, more preferably hematopoietic stem or progenitor cell, and even more preferably CD34+ cells.

As used herein, the terms “individual” and “subject” are often used interchangeably and refer to any animal that exhibits a symptom of XLA that can be treated with the reprogrammed nucleases, genome editing compositions, gene therapy vectors, genome editing vectors, genome edited cells, and methods contemplated elsewhere herein. Suitable subjects (e.g., patients) include laboratory animals (such as mouse, rat, rabbit, or guinea pig), farm animals, and domestic animals or pets (such as a cat or dog). Non-human primates and, preferably, human subjects, are included. Typical subjects include human patients that have, have been diagnosed with, or are at risk of having XLA.

As used herein, the term “patient” refers to a subject that has been diagnosed with XLA that can be treated with the reprogrammed nucleases, genome editing compositions, gene therapy vectors, genome editing vectors, genome edited cells, and methods contemplated elsewhere herein.

As used herein “treatment” or “treating,” includes any beneficial or desirable effect on the symptoms or pathology of XLA, and may include even minimal reductions in one or more measurable markers of XLA. Treatment can optionally involve delaying of the progression of XLA. “Treatment” does not necessarily indicate complete eradication or cure of XLA, or associated symptoms thereof.

As used herein, “prevent,” and similar words such as “prevention,” “prevented,” “preventing” etc., indicate an approach for preventing, inhibiting, or reducing the likelihood of the occurrence or recurrence of, XLA. It also refers to delaying the onset or recurrence of XLA or delaying the occurrence or recurrence of XLA. As used herein, “prevention” and similar words also includes reducing the intensity, effect, symptoms and/or burden of XLA prior to its onset or recurrence.

As used herein, the phrase “ameliorating at least one symptom of” refers to decreasing one or more symptoms of XLA. In particular embodiments, one or more symptoms of XLA that are ameliorated include, but are not limited to, common infections including but not limited to bronchitis (airway infection), chronic diarrhea, conjunctivitis (eye infection), otitis media (middle ear infection), pneumonia (lung infection), sinusitis (sinus infection), skin infections, upper respiratory tract infections; infections due to bacteria, viruses, and other microbes; and bacterial infections including, but not limited to, Haemophilus influenzae, pneumococci (Streptococcus pneumoniae), and staphylococci infections.

As used herein, the term “amount” refers to “an amount effective” or “an effective amount” of a nuclease variant, genome editing composition, or genome edited cell sufficient to achieve a beneficial or desired prophylactic or therapeutic result, including clinical results.

A “prophylactically effective amount” refers to an amount of a nuclease variant, genome editing composition, or genome edited cell sufficient to achieve the desired prophylactic result. Typically but not necessarily, since a prophylactic dose is used in subjects prior to or at an earlier stage of disease, the prophylactically effective amount is less than the therapeutically effective amount.

A “therapeutically effective amount” of a nuclease variant, genome editing composition, or genome edited cell may vary according to factors such as the disease state, age, sex, and weight of the individual, and the ability to elicit a desired response in the individual. A therapeutically effective amount is also one in which any toxic or detrimental effects are outweighed by the therapeutically beneficial effects. The term “therapeutically effective amount” includes an amount that is effective to “treat” a subject (e.g., a patient). When a therapeutic amount is indicated, the precise amount of the compositions contemplated in particular embodiments, to be administered, can be determined by a physician in view of the specification and with consideration of individual differences in age, weight, tumor size, extent of infection or metastasis, and condition of the patient (subject).

The genome edited cells may be administered as part of a bone marrow or cord blood transplant in an individual that has or has not undergone bone marrow ablative therapy. In one embodiment, genome edited cells contemplated herein are administered in a bone marrow transplant to an individual that has undergone chemoablative or radioablative bone marrow therapy.

In one embodiment, a dose of genome edited cells is delivered to a subject intravenously. In preferred embodiments, genome edited hematopoietic stem cells are intravenously administered to a subject.

In one illustrative embodiment, the effective amount of genome edited cells provided to a subject is at least 2×10⁶ cells/kg, at least 3×10⁶ cells/kg, at least 4×10⁶ cells/kg, at least 5×10⁶ cells/kg, at least 6×10⁶ cells/kg, at least 7×10⁶ cells/kg, at least 8×10⁶ cells/kg, at least 9×10⁶ cells/kg, or at least 10×10⁶ cells/kg, or more cells/kg, including all intervening doses of cells.

In another illustrative embodiment, the effective amount of genome edited cells provided to a subject is about 2×10⁶ cells/kg, about 3×10⁶ cells/kg, about 4×10⁶ cells/kg, about 5×10⁶ cells/kg, about 6×10⁶ cells/kg, about 7×10⁶ cells/kg, about 8×10⁶ cells/kg, about 9×10⁶ cells/kg, or about 10×10⁶ cells/kg, or more cells/kg, including all intervening doses of cells.

In another illustrative embodiment, the effective amount of genome edited cells provided to a subject is from about 2×10⁶ cells/kg to about 10×10⁶ cells/kg, about 3×10⁶ cells/kg to about 10×10⁶ cells/kg, about 4×10⁶ cells/kg to about 10×10⁶ cells/kg, about 5×10⁶ cells/kg to about 10×10⁶ cells/kg, 2×10⁶ cells/kg to about 6×10⁶ cells/kg, 2×10⁶ cells/kg to about 7×10⁶ cells/kg, 2×10⁶ cells/kg to about 8×10⁶ cells/kg, 3×10⁶ cells/kg to about 6×10⁶ cells/kg, 3×10⁶ cells/kg to about 7×10⁶ cells/kg, 3×10⁶ cells/kg to about 8×10⁶ cells/kg, 4×10⁶ cells/kg to about 6×10⁶ cells/kg, 4×10⁶ cells/kg to about 7×10⁶ cells/kg, 4×10⁶ cells/kg to about 8×10⁶ cells/kg, 5×10⁶ cells/kg to about 6×10⁶ cells/kg, 5×10⁶ cells/kg to about 7×10⁶ cells/kg, 5×10⁶ cells/kg to about 8×10⁶ cells/kg, or 6×10⁶ cells/kg to about 8×10⁶ cells/kg, including all intervening doses of cells.

Some variation in dosage will necessarily occur depending on the condition of the subject being treated. The person responsible for administration will, in any event, determine the appropriate dose for the individual subject.

In particular embodiments, a genome edited cell therapy is used to treat, prevent, or ameliorate XLA, or a condition associated therewith, comprising administering to subject having one or more mutations and/or deletions in a BTK gene that results in little or no endogenous BTK expression, a therapeutically effective amount of the genome edited cells contemplated herein. In one embodiment, the genome edited cell therapy lacks functional endogenous BTK expression, but comprises an exogenous polynucleotide encoding a functional BTK polypeptide.

In various embodiments, a subject is administered an amount of genome edited cells comprising an exogenous polynucleotide encoding a functional BTK polypeptide, effective to increase BTK expression in the subject. In particular embodiments, the amount of BTK expression from the exogenous polynucleotide in genome edited cells comprising one or more deleterious mutations or deletions in a BTK gene is increased at least about 10%, at least about 20%, at least about 30%, at least about 40%, at least about 50%, at least about 60%, at least about 70%, at least about 80%, at least about 90%, at least about 100%, at least about 2-fold, at least about 5-fold, at least about 10-fold, at least about 50-fold, at least about 100-fold, at least about 200-fold, at least about 300-fold, at least about 400-fold, at least about 500-fold, or at least about 1000-fold, or more compared endogenous BTK expression.

One of ordinary skill in the art would be able to use routine methods in order to determine the appropriate route of administration and the correct dosage of an effective amount of a composition comprising genome edited cells contemplated herein. It would also be known to those having ordinary skill in the art to recognize that in certain therapies, multiple administrations of pharmaceutical compositions contemplated herein may be required to effect therapy.

One of the prime methods used to treat subjects amenable to treatment with genome edited hematopoietic stem and progenitor cell therapies is blood transfusion. Thus, one of the chief goals of the compositions and methods contemplated herein is to reduce the number of, or eliminate the need for, transfusions.

In particular embodiments, the drug product is administered once.

In certain embodiments, the drug product is administered 1, 2, 3, 4, 5, 6, 7, 8, 9, or 10 or more times over a span of 1 year, 2 years, 5, years, 10 years, or more.

All publications, patent applications, and issued patents cited in this specification are herein incorporated by reference as if each individual publication, patent application, or issued patent were specifically and individually indicated to be incorporated by reference.

Although the foregoing embodiments have been described in some detail by way of illustration and example for purposes of clarity of understanding, it will be readily apparent to one of ordinary skill in the art in light of the teachings contemplated herein that certain changes and modifications may be made thereto without departing from the spirit or scope of the appended claims. The following examples are provided by way of illustration only and not by way of limitation. Those of skill in the art will readily recognize a variety of noncritical parameters that could be changed or modified to yield essentially similar results.

### EXAMPLES

### Example 1

### Reprogramming I-OnuI to a Target Site in Intron 2 of the Human BTK Gene

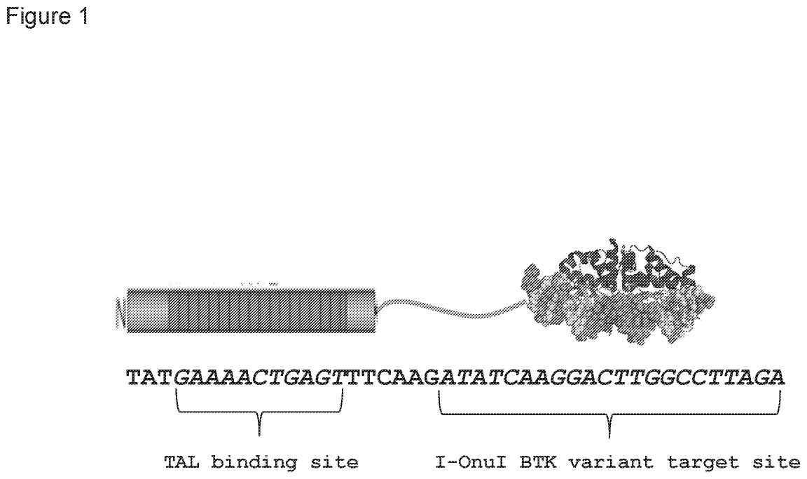

I-OnuI was reprogrammed to a target site in the second intron of the human Bruton's tyrosine kinase (BTK) gene (FIG. 1) by constructing modular libraries containing variable amino acid residues in the DNA recognition interface. To construct the variants, degenerate codons were incorporated into I-OnuI DNA binding domains using oligonucleotides. The oligonucleotides encoding the degenerate codons were used as PCR templates to generate variant libraries by gap recombination in the yeast strain S. cerevisiae. Each variant library spanned either the N- or C-terminal I-OnuI DNA recognition domain and contained ˜10⁷ to 10⁸ unique transformants. The resulting surface display libraries were screened by flow cytometry for cleavage activity against target sites comprising the corresponding domains' “half-sites.”

Yeast displaying the N- and C-terminal domain reprogrammed I-OnuI HEs were purified and the plasmid DNA was extracted. PCR reactions were performed to amplify the reprogrammed domains, which were subsequently transformed into S. cerevisiae to create a library of reprogrammed domain combinations. Fully reprogrammed I-OnuI variants that recognize the complete target site (SEQ ID NO: 24) present in the BTK gene were identified from this library and purified.

### Example 2

### Reprogrammed I-OnuI Homing Endonucleases that Efficiently Target Intron 2 of the Human BTK Gene

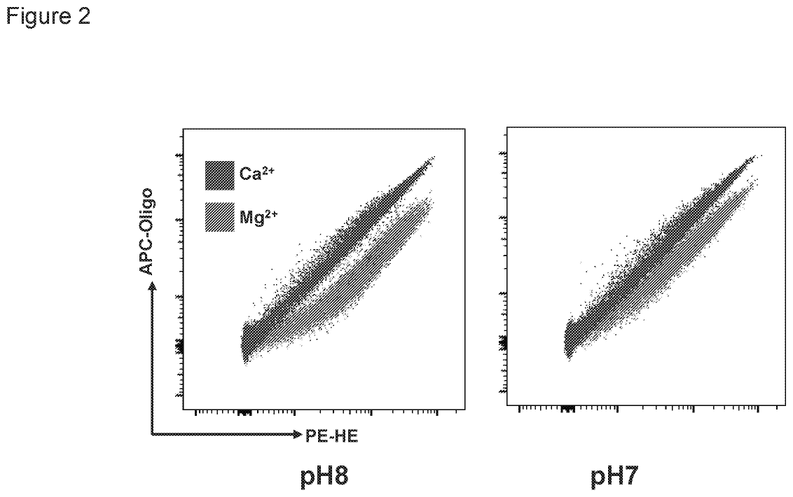

A secondary I-OnuI variant library was generated by performing random mutagenesis on the reprogrammed I-OnuI HEs that target the BTK gene target site, identified in the initial screen. In addition, display-based flow sorting was performed under cleavage conditions of pH 7 and pH 8 in an effort to isolate variants with improved catalytic efficiency. FIG. 2.

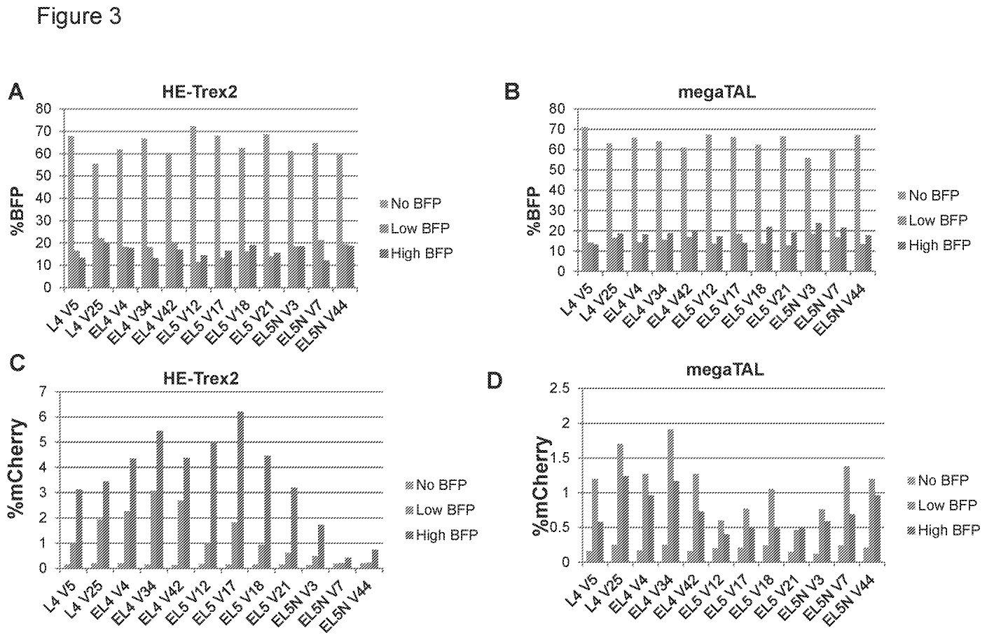

The activity of reprogrammed I-OnuI HEs that target intron 2 in the BTK gene was measured using a chromosomally integrated fluorescent reporter system (Certo et. al., 2011). Fully reprogrammed I-OnuI HEs that bind and cleave the BTK target sequence were cloned into mammalian expression plasmids reformatting the HEs as TREX2 fusions or megaTALs and linked to BFP (to normalize expression) and then individually transfected into a HEK 293T fibroblast cell line that was reprogrammed to contain the BTK target sequence upstream of an out-of-frame gene encoding the fluorescent mCherry protein. Cleavage of the embedded target site by the HE and the subsequent accumulation of small insertions or deletions, caused by DNA repair via the non-homologous end joining (NHEJ) pathway, results in approximately one out of three repaired loci placing the fluorescent reporter gene back “in-frame”. mCherry fluorescence is therefore a readout of endonuclease activity at the chromosomally embedded target sequence. Expression levels of the variants were consistent among each other. FIG. 3A and FIG. 3B. The fully reprogrammed I-OnuI HEs that bind and cleave the BTK target site showed mCherry expression in a cellular chromosomal context. FIGS. 3C and 3D.

### Example 3

### Cleavage Efficiency of Selected MegaTALs Variants

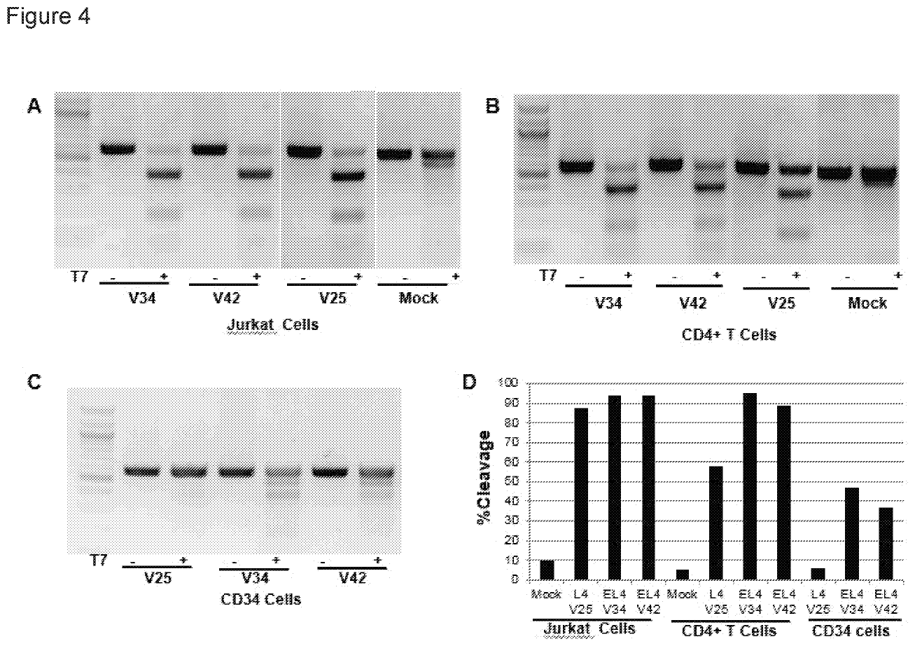

Three I-OnuI BTK megaTALs (MTBTK_L4_V25, MTBTK_EL4_V34, and MTBTK_EL4_V42; SEQ ID NOs: 18-20, resp.) were subcloned into a vector with T7 promoter for mRNA production. Jurkat cells (FIG. 4A), human primary CD4⁺ T cells (FIG. 4B), and human CD34 cells (FIG. 4C) were transfected with megaTAL mRNA with a NEON electroporation device. Seventy-two hours after transfection, genomic DNA was collected from the cells and the BTK target locus was amplified by PCR. The cleavage efficiency of the BTK site was measured by T7 endonuclease assay, which detect megaTAL-induced non-homologous end joining (NHEJ) mutations. The DNA bands intensity was quantified by ImageJ software (National Institutes of Health). Cleavage percentage was calculated as (T7−−T7+)/T7−*100% (FIG. 4D). Data shown is representative of at least two independent experiments.

### Example 4

### BTK MegaTALs Induce Homology Directed Repair (HDR)

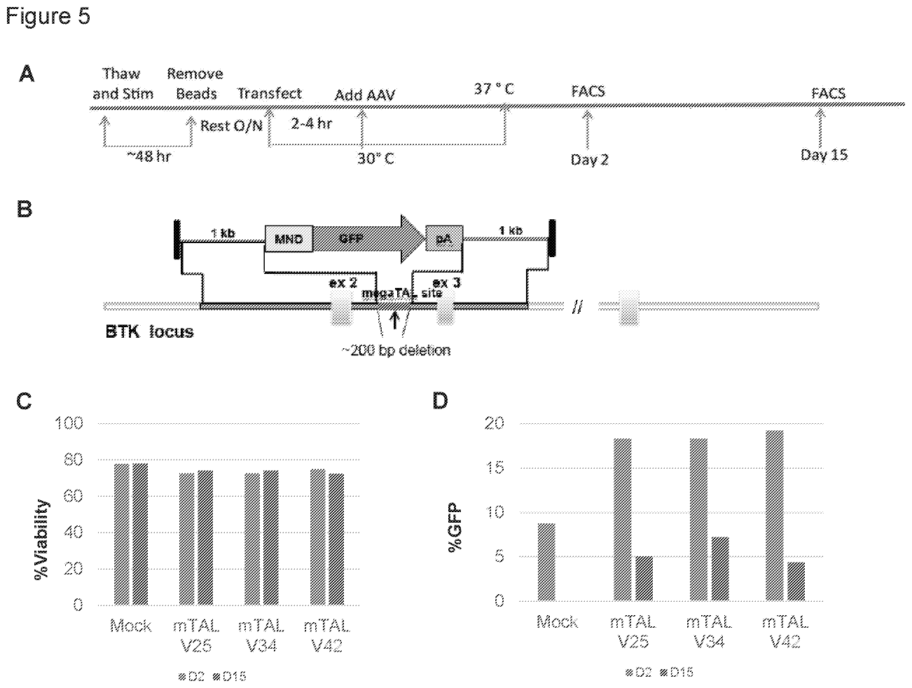

Three I-OnuI BTK megaTAL mRAN (MTBTK_L4_V25, MTBTK_EL4_V34, and MTBTK_EL4_V42) were electroplated into human primary CD4+ T cells to compare their ability to induce HDR using AAV as donor template. See, e.g., SEQ ID NO: 34. FIG. 5A is the illustration of experimental approach. Percentage of cell viability (based on flow cytometry forward and side scatter gating) and HDR (based on GFP expression) were measured by flow cytometry at day 2 and day 15 after mRNA transfection and AAV transduction. FIG. 5B shows the structure of GFP-expressing AAV donor template. The 200 bp sequence with megaTAL cleavage site is deleted between AAV 5′ end homology arm and 3′ end homology end. FIG. 5C shows viability of CD4+ T cells at day 2 and day 15, and FIG. 5D shows GFP expression at day 2 and day 15 after mRNA transfection and AAV transduction. Data shown is representative of two independent experiments.

### Example 5

### BTK MegaTALs MTBTK_EL4_V34 Induces High Efficiency HDR

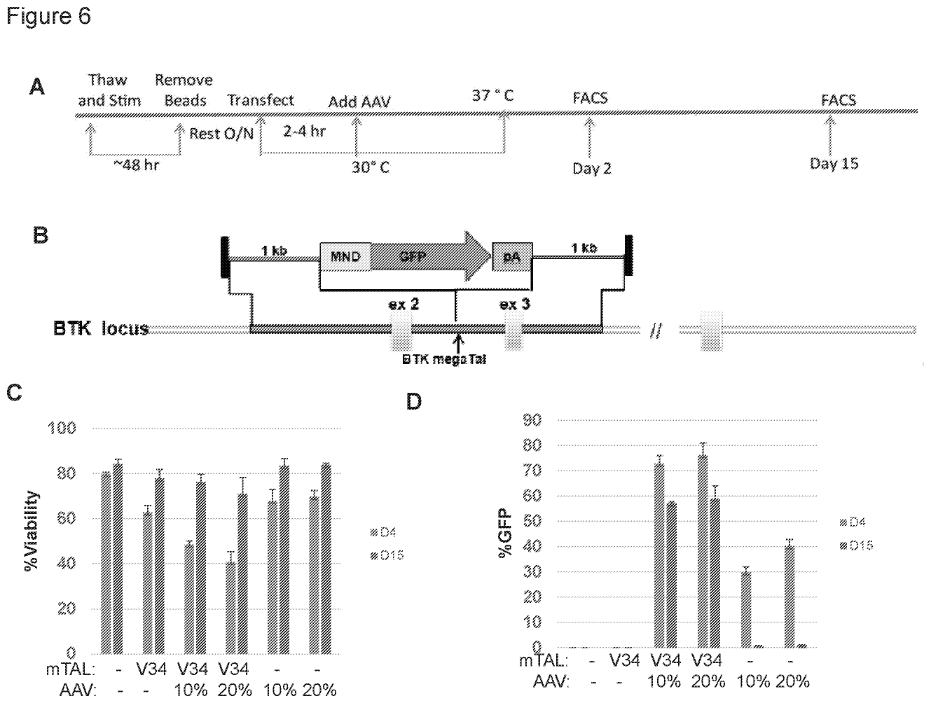

MegaTAL-induced HDR was increased in human primary CD4⁺ cell by using an AAV construct with a megaTAL cleavage site in the 3′ homology arm. A GFP expression cassette was inserted into the middle of the BTK OnuI HE cleavage site to generate a non-cleavable donor template without deletion of the cleavage site (FIG. 6B). See, e.g., SEQ ID NO: 33. MTBTK_EL4_V34-induced HDR and cell viability were measured in the presence of 10% and 20% cell culture volume AAV donor, at day 2 and day 15 after transfection. AAV transduction without megaTAL mRNA transfection was used as control to measure non-HDR GFP background. Data shown is the average of two different donors.

### Example 6

### BTK megaTALs MTBTK_EL4_V34 Induces High Efficiency HDR in Primary Human CD34⁺ Cells

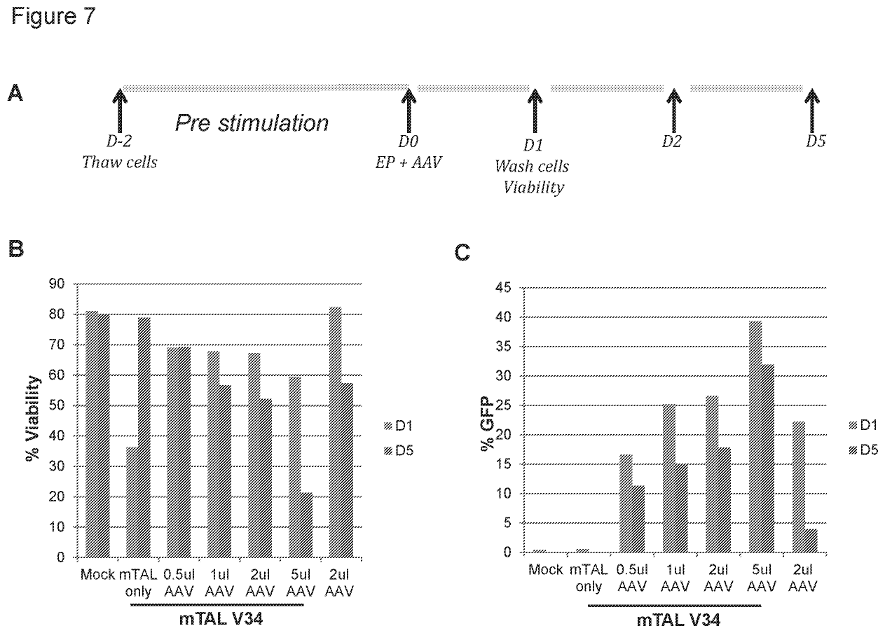

MegaTAL-induced HDR was increased in human primary CD34⁺ cells by using an AAV construct with a megaTAL cleavage site in the 3′ homology arm. A GFP expression cassette was inserted into the middle of the BTK OnuI HE cleavage site to generate a non-cleavable donor template without deletion of the cleavage site. MTBTK_EL4_V34-induced HDR and cell viability were measured in the presence of increasing amounts of AAV donor, at day land day 5 after transfection. AAV transduction without megaTAL mRNA transfection was used as control to measure non-HDR GFP background. (FIGS. 7A-7C).

### Example 7

### Viability of BTK-Edited Primary Human CD34⁺ Cells

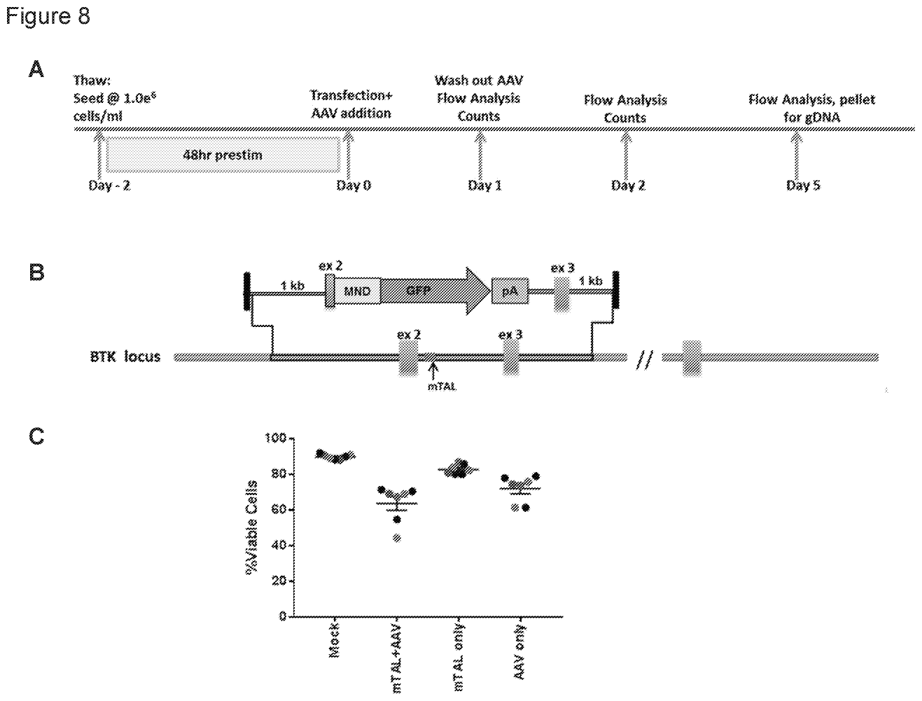

MegaTAL MTBTK_EL4_V34 was electroplated into human primary CD34⁺ cells along with a rAAV6 donor template. FIG. 8A illustrates the experimental approach. The HDR strategy is shown in FIG. 8B.

Adult human mobilized CD34+ cells were cultured in SCGM media supplemented with TPO, SCF, FLT3L and IL6 (100 ng/ml) for 48 hours, followed by electroporation of 1 μg megaTAL MTBTK_EL4_V34 mRNA and addition of rAAV6 at 3% of the total culture volume, electroporation of megaTAL only, or addition of rAAV6 only. Cell viability was assessed by forward and side scatter one day post editing. FIG. 8C. Treatment of CD34+ cells with megaTAL only resulted in a minimal decrease in overall cell viability at Day 1 post transfection. Co-delivery of megaTAL and rAAV6 resulted in viability rates that were equivalent to rAAV6 only treatement. These findings indicate that BTK mTAL is well tolerated by CD34+ HSC.

### Example 8

### HDR in BTK-Edited Primary Human CD34+ Cells

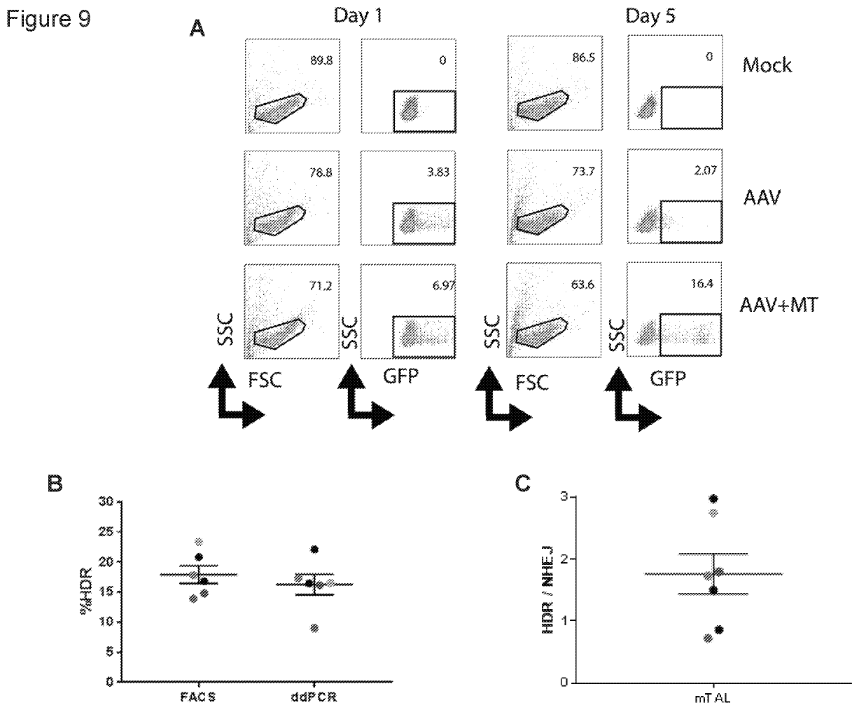

Percent homology directed repair (% HDR) in CD34⁺ cells by FACS (GFP+) was compared to % HDR by droplet digital PCR (ddPCR) 5 days post editing. Adult human mobilized CD34⁺ cells were cultured in SCGM media supplemented with TPO, SCF, FLT3L and IL6 (100 ng/ml) for 48 hours, followed by electroporation. Cells were transfected with 1 μg of megaTAL MTBTK_EL4_V34 mRNA and AAV was added at a culture volume of 3%. GFP expression was assessed by flow cytometry (FACS) on day 5 following which genomic DNA was extracted. Cell viabilities and GFP expression were assessed on days 1 and 5 post editing using flow cytometry. FIG. 9A.

To assess editing rates at the genome level, “in-out” droplet digital PCR was performed with the forward primer binding within the AAV insert and a reverse primer that binds the BTK locus outside the region of homology. A control amplicon of similar size was generated for the CCR5 gene to serve as a control. All reactions were performed in duplicate. The PCR reactions were partitioned into droplets using a QX200 Droplet Generator (Bio-Rad). Amplification was performed using ddPCR Supermix for Probes without UTP (Bio-Rad), 900 nM of primers, 250 nM of Probe and 50 ng of genomic DNA. Droplets were analyzed on the QX200 Droplet Digital PCR System (Bio-Rad) using QuantaSoft software (Bio-Rad). Colors represent independent CD34⁺ donors. Data are presented as mean±SEM. FIG. 9B.

The ratio of HDR to NHEJ in the edited CD34⁺ cells was also measured. NHEJ rates were determined by PCR amplification of the region around the cut site, gel extraction, and ICE (Inference of CRISPR Edits) analysis. The ratio of HDR vs NHEJ in shown in FIG. 9C.

### Example 9

### Colony Formation in BTK-Edited Primary Human CD34⁺ Cells

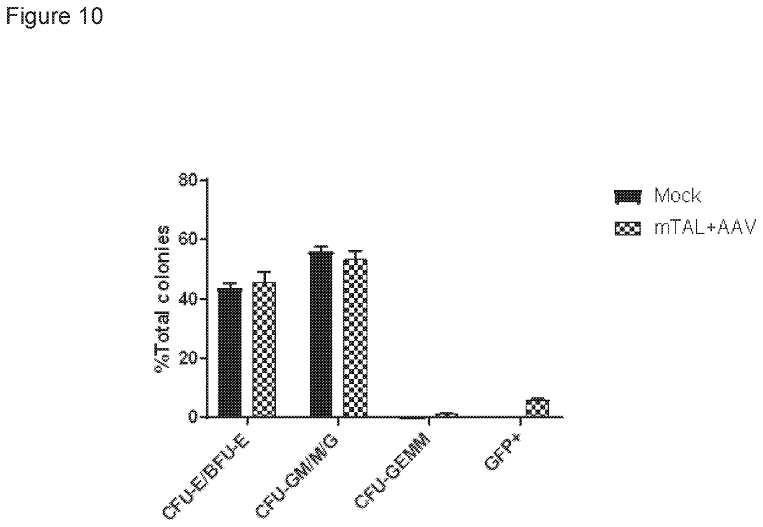

Adult human mobilized CD34⁺ cells were cultured in SCGM media supplemented with TPO, SCF, FLT3L and IL6 (100 ng/mL) for 48 hours, followed by electroporation. Cells were transfected with 1 μg of megaTAL MTBTK_EL4_V34 mRNA and AAV was added at a culture volume of 3%. mTAL edited and mock cells were plated one day post editing onto Methocult media for colony formation unit (CFU) assay. Briefly, 500 cells were plated in duplicate in Methocult H4034 media (Stemcell Technologies), incubated at 37° C. for 12-14 days and colonies enumerated based on their morphology and GFP expression. Data shown in FIG. 10 is from 3 independent donors and presented as mean±SEM.

### Example 10

### Using HDR to Express a Codon Optimized BTK cDNA

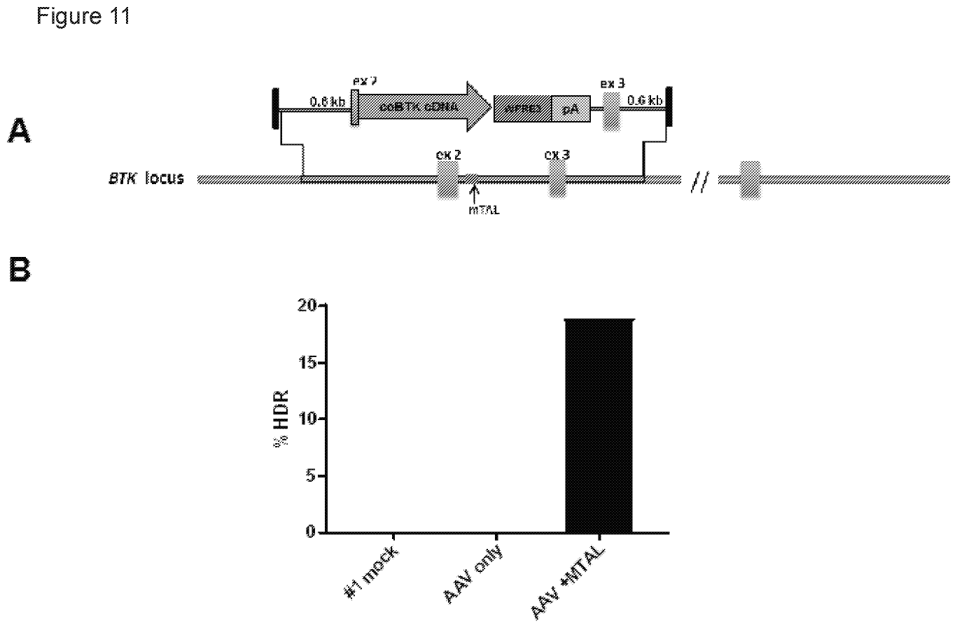

Adult human mobilized CD34⁺ cells were cultured in SCGM media supplemented with TPO, SCF, FLT3L and IL6 (100 ng/mL) for 48 hours, followed by electroporation. Cells were transfected with 1 μg of megaTAL MTBTK_EL4_V34 mRNA followed by addition of a rAAV6 cDNA targeting vector encoding a codon optimized human BTK CDNA, a truncated wood chuck hepatitis post-transcriptional regulatory element (WPRE3) and SV40 polyadenylation signal (AAV.coBTK, see, e.g., SEQ ID NO: 35). FIG. 11A. Genomic DNA extracted 5 days later.

HDR editing rates were determined. “In-out” droplet digital PCR was performed with the forward primer binding within the AAV insert and a reverse primer that binds the BTK locus outside the region of homology. A control amplicon of similar size was generated for the CCR5 gene to serve as a control. All reactions were performed in duplicate. The PCR reactions were partitioned into droplets using a QX200 Droplet Generator (Bio-Rad). Amplification was performed using ddPCR Supermix for Probes without UTP (Bio-Rad), 900 nM of primers, 250 nM of Probe and 50 ng of genomic DNA. Droplets were analyzed on the QX200 Droplet Digital PCR System (Bio-Rad) using QuantaSoft software (Bio-Rad). % HDR is shown FIG. 11B.

### Example 11

### BTK MegaTALs MTBTK_EL4_V34 Induces High Efficiency HDR In Primary Human CD34⁺ Cells

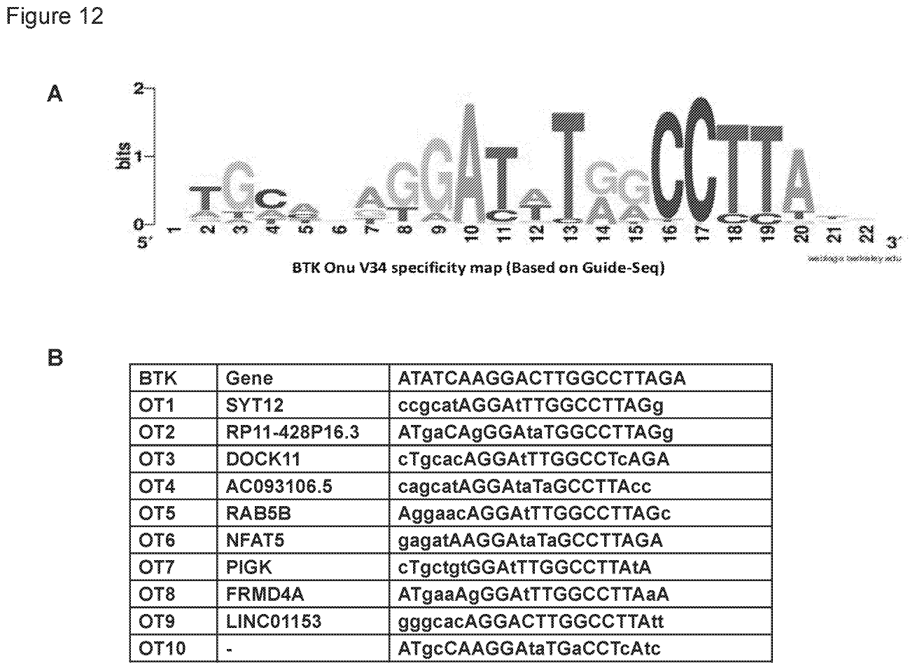

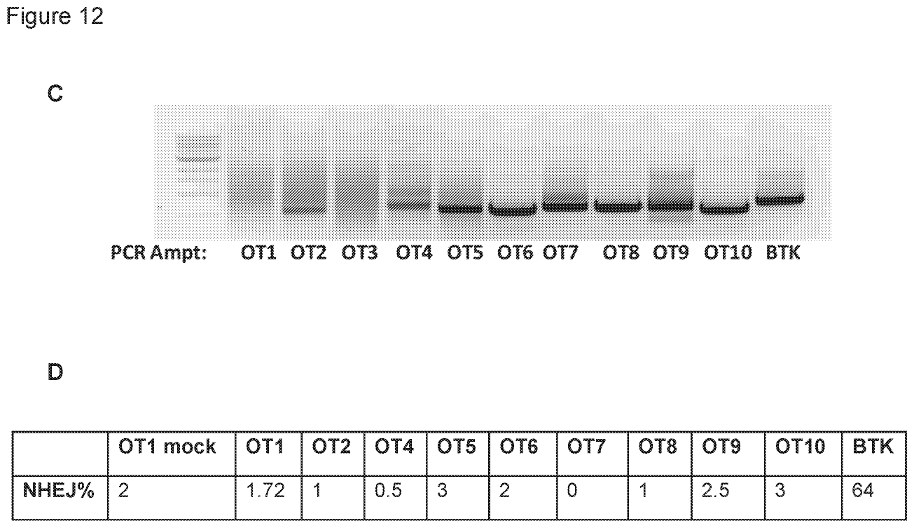

Adult human T cells were electroporated with 1 μg of megaTAL MTBTK_EL4_V34 mRNA. Off-target sites were identified using GUIDE-Seq. megaTAL specificity (FIG. 12A) was determined using GUIDE-Seq data. NHEJ rates for putative off-target sites were analysis using ICE (Inference of CRISPR Edits) analysis. The top ten putative off-target sites predicted by GUIDE-Seq (FIG. 12B) were PCR amplified from genomic DNA of the edited CD4⁺ T cells. FIG. 12C. Although there was significant predicted cleavage at some off-target sites in GUIDE-Seq experiment (up to 50% of on-target cleavage for OT1 site), the level of NHEJ for top-ranked off-target sites is not significantly higher than the background reading in this assay (OT1 from mock treated cells; up to 3%). In contrast, the on-target NHEJ rate for the BTK locus site was 64% by ICE analysis. FIG. 12D.

In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

What is claimed is:

1. A polypeptide comprising a homing endonuclease (HE) variant that cleaves a target site in the human Bruton's tyrosine kinase (BTK) gene.

2. The polypeptide of claim 1, wherein the HE variant is an LAGLIDADG homing endonuclease (LHE) variant.

3. The polypeptide of claim 1, or claim 2, wherein the polypeptide comprises a biologically active fragment of the HE variant.

4. The polypeptide of claim 3, wherein the biologically active fragment lacks the 1, 2, 3, 4, 5, 6, 7, or 8 N-terminal amino acids compared to a corresponding wild type HE.

5. The polypeptide of claim 4, wherein the biologically active fragment lacks the 4 N-terminal amino acids compared to a corresponding wild type HE.

6. The polypeptide of claim 4, wherein the biologically active fragment lacks the 8 N-terminal amino acids compared to a corresponding wild type HE.

7. The polypeptide of claim 3, wherein the biologically active fragment lacks the 1, 2, 3, 4, or 5 C-terminal amino acids compared to a corresponding wild type HE.

8. The polypeptide of claim 7, wherein the biologically active fragment lacks the C-terminal amino acid compared to a corresponding wild type HE.

9. The polypeptide of claim 7, wherein the biologically active fragment lacks the 2 C-terminal amino acids compared to a corresponding wild type HE.

10. The polypeptide of any one of claims 1 to 9, wherein the HE variant is a variant of an LHE selected from the group consisting of: I-AabMI, I-AaeMI, I-AniI, I-ApaMI, I-CapIII, I-CapIV, I-CkaMI, I-CpaMI, I-CpaMII, I-CpaMIII, I-CpaMIV, I-CpaMV, I-CpaV, I-CraMI, I-EjeMI, I-GpeMI, I-GpiI, I-GzeMI, I-GzeMII, I-GzeMIII, I-HjeMI, I-LtrII, I-LtrI, I-LtrWI, I-MpeMI, I-MveMI, I-NcrII, I-Ncrl, I-NcrMI, I-OheMI, I-OnuI, I-OsoMI, I-OsoMII, I-OsoMIII, I-OsoMIV, I-PanMI, I-PanMII, I-PanMIII, I-PnoMI, I-SceI, I-ScuMI, I-SmaMI, I-SscMI, and I-Vdi141I.

11. The polypeptide of any one of claims 1 to 10, wherein the HE variant is a variant of an LHE selected from the group consisting of: I-CpaMI, I-HjeMI, I-OnuI, I-PanMI, and I-SmaMI.

12. The polypeptide of any one of claims 1 to 11, wherein the HE variant is an I-OnuI LHE variant.

13. The polypeptide of any one of claims 1 to 12, wherein the HE variant comprises one or more amino acid substitutions at amino acid positions selected from the group consisting of: 24, 26, 28, 30, 32, 34, 35, 36, 37, 38, 40, 42, 44, 46, 48, 68, 70, 72, 75, 76, 78, 80, 82, 180, 182, 184, 186, 188, 189, 190, 191, 192, 193, 195, 197, 199, 201, 203, 223, 225, 227, 229, 231, 232, 234, 236, 238, and 240 of an I-OnuI LHE amino acid sequence as set forth in SEQ ID NOs: 1-5, or a biologically active fragment thereof.

14. The polypeptide of any one of claims 1 to 13, wherein the HE variant comprises at least 5, at least 15, preferably at least 25, more preferably at least 35, or even more preferably at least 40 or more amino acid substitutions at amino acid positions selected from the group consisting of: 24, 26, 28, 30, 32, 34, 35, 36, 37, 38, 40, 42, 44, 46, 48, 68, 70, 72, 75, 76, 78, 80, 82, 180, 182, 184, 186, 188, 189, 190, 191, 192, 193, 195, 197, 199, 201, 203, 223, 225, 227, 229, 231, 232, 234, 236, 238, and 240 of an I-OnuI LHE amino acid sequence as set forth in SEQ ID NOs: 1-5, or a biologically active fragment thereof.

15. The polypeptide of any one of claims 1 to 14, wherein the HE variant comprises one or more amino acid substitutions at amino acid positions selected from the group consisting of: 24, 26, 28, 30, 32, 34, 35, 36, 37, 38, 40, 42, 44, 46, 48, 61, 68, 70, 72, 75, 76, 78, 80, 82, 85, 116, 135, 138, 143, 147, 159, 164, 168, 178, 180, 182, 184, 186, 188, 189, 190, 191, 192, 193, 195, 197, 199, 201, 203, 210, 223, 225, 227, 229, 231, 232, 234, 236, 238, 240, and 246 of an I-OnuI LHE amino acid sequence as set forth in SEQ ID NOs: 1-5, or a biologically active fragment thereof.

16. The polypeptide of any one of claims 1 to 15, wherein the HE variant comprises at least 5, at least 15, preferably at least 25, more preferably at least 35, or even more preferably at least 40 or more amino acid substitutions at amino acid positions selected from the group consisting of: 24, 26, 28, 30, 32, 34, 35, 36, 37, 38, 40, 42, 44, 46, 48, 61, 68, 70, 72, 75, 76, 78, 80, 82, 85, 116, 135, 138, 143, 147, 159, 164, 168, 178, 180, 182, 184, 186, 188, 189, 190, 191, 192, 193, 195, 197, 199, 201, 203, 210, 223, 225, 227, 229, 231, 232, 234, 236, 238, 240, and 246 of an I-OnuI LHE amino acid sequence as set forth in SEQ ID NOs: 1-17, or a biologically active fragment thereof.

17. The polypeptide of any one of claims 1 to 16, wherein the HE variant comprises at least 5, at least 15, preferably at least 25, more preferably at least 35, or even more preferably at least 40 or more amino acid substitutions at amino acid positions selected from the group consisting of: 24, 26, 28, 30, 32, 34, 35, 36, 37, 38, 40, 42, 44, 46, 48, 61, 68, 70, 72, 75, 76, 78, 80, 82, 85, 116, 135, 138, 143, 147, 159, 164, 168, 178, 180, 182, 184, 186, 188, 189, 190, 191, 192, 193, 195, 197, 199, 201, 203, 210, 223, 225, 227, 229, 231, 232, 234, 236, 238, 240, and 246 of an I-OnuI LHE amino acid sequence as set forth in SEQ ID NOs: 1-5, or a biologically active fragment thereof.

18. The polypeptide of any one of claims 1 to 17, wherein the HE variant comprises at least 5, at least 15, preferably at least 25, more preferably at least 35, or even more preferably at least 40 or more of the following amino acid substitutions: S24W, L26M, L26S, R28V, R28D, N32S, K34T, S35V, S36K, S40R, E42L, G44S, Q46G, T48E, Q61R, V68K, A70S, A70R, N75H, N75R, A76Y, S78R, K80T, T82S, E85G, V 116L, K135R, L138M, T143N, K147E, S159P, I161V, N164S, F168L, E178D, C180S, C180T, F182Y, I186V, S188G, S190N, K191T, L192T, G193R, Q195T, Q195Y, S201Q, S201G, N210Y, K225L, K229V, F232R, W234F, D236Q, V238R, and N246K, in reference to an I-OnuI LHE amino acid sequence as set forth in SEQ ID NOs: 1-5, or a biologically active fragment thereof.

19. The polypeptide of any one of claims 1 to 18, wherein the HE variant comprises at least 5, at least 15, preferably at least 25, more preferably at least 35, or even more preferably at least 40 or more of the following amino acid substitutions: S24W, R28V, N32S, K34T, S35V, S36K, S40R, E42L, G44S, Q46G, T48E, V68K, A70S, N75H, A76Y, S78R, K80T, T82S, V116L, L138M, T143N, S159P, F168L, E178D, C180S, F182Y, I186V, S188G, S190N, K191T, L192T, G193R, Q195T, S201Q, K225L, K229V, F232R, W234F, D236Q, and V238R, in reference to an I-OnuI LHE amino acid sequence as set forth in SEQ ID NOs: 1-5, or a biologically active fragment thereof.

20. The polypeptide of any one of claims 1 to 18, wherein the HE variant comprises at least 5, at least 15, preferably at least 25, more preferably at least 35, or even more preferably at least 40 or more of the following amino acid substitutions: S24W, R28V, N32S, K34T, S35V, S36K, S40R, E42L, G44S, Q46G, T48E, V68K, A70S, N75H, A76Y, S78R, K80T, T82S, V116L, K135R, L138M, T143N, S159P, F168L, E178D, C180S, F182Y, I186V, S188G, S190N, K191T, L192T, G193R, Q195T, S201Q, K225L, K229V, F232R, W234F, D236Q, V238R, and N246K, in reference to an I-OnuI LHE amino acid sequence as set forth in SEQ ID NOs: 1-5, or a biologically active fragment thereof.

21. The polypeptide of any one of claims 1 to 18, wherein the HE variant comprises at least 5, at least 15, preferably at least 25, more preferably at least 35, or even more preferably at least 40 or more of the following amino acid substitutions: S24W, R28D, N32S, K34T, S35V, S36K, S40R, E42L, G44S, Q46G, T48E, V68K, A70R, N75R, A76Y, K80T, T82S, V116L, L138M, T143N, S159P, N164S, F168L, E178D, C180S, F182Y, I186V, S188G, S190N, K191T, L192T, G193R, Q195T, S201Q, N210Y, K225L, K229V, F232R, W234F, D236Q, and V238R, in reference to an I-OnuI LHE amino acid sequence as set forth in SEQ ID NOs: 1-5, or a biologically active fragment thereof.

22. The polypeptide of any one of claims 1 to 18, wherein the HE variant comprises at least 5, at least 15, preferably at least 25, more preferably at least 35, or even more preferably at least 40 or more of the following amino acid substitutions: S24W, R28V, N32S, K34T, S35V, S36K, S40R, E42L, G44S, Q46G, T48E, V68K, A70S, N75H, A76Y, S78R, K80T, T82S, L138M, T143N, S159P, F168L, E178D, C180T, F182Y, S188G, S190N, K191T, L192T, G193R, Q195Y, S201G, K225L, K229V, F232R, W234F, D236Q, and V238R, in reference to an I-OnuI LHE amino acid sequence as set forth in SEQ ID NOs: 1-5, or a biologically active fragment thereof.

23. The polypeptide of any one of claims 1 to 18, wherein the HE variant comprises at least 5, at least 15, preferably at least 25, more preferably at least 35, or even more preferably at least 40 or more of the following amino acid substitutions: S24W, R28V, R28D, K34T, S35V, S36K, S40R, E42L, G44S, Q46G, T48E, V68K, A70S, N75H, A76Y, S78R, K80T, T82S, V116L, L138M, T143N, S159P, F168L, E178D, C180S, F182Y, S188G, S190N, K191T, L192T, G193R, Q195T, S201Q, K225L, K229V, F232R, W234F, D236Q, and V238R, in reference to an I-OnuI LHE amino acid sequence as set forth in SEQ ID NOs: 1-5, or a biologically active fragment thereof.

24. The polypeptide of any one of claims 1 to 18, wherein the HE variant comprises at least 5, at least 15, preferably at least 25, more preferably at least 35, or even more preferably at least 40 or more of the following amino acid substitutions: S24W, R28D, N32S, K34T, S35V, S36K, S40R, E42L, G44S, Q46G, T48E, V68K, A70S, N75H, A76Y, S78R, K80T, T82S, V116L, L138M, T143N, S159P, F168L, E178D, C180T, F182Y, S188G, S190N, K191T, L192T, G193R, Q195Y, S201G, K225L, K229V, F232R, W234F, D236Q, and V238R, in reference to an I-OnuI LHE amino acid sequence as set forth in SEQ ID NOs: 1-5, or a biologically active fragment thereof.

25. The polypeptide of any one of claims 1 to 18, wherein the HE variant comprises at least 5, at least 15, preferably at least 25, more preferably at least 35, or even more preferably at least 40 or more of the following amino acid substitutions: S24W, L26M, R28D, N32S, K34T, S35V, S36K, S40R, E42L, G44S, Q46G, T48E, V68K, A70R, N75R, A76Y, K80T, T82S, V116L, L138M, T143N, K147E, S159P, F168L, E178D, C180T, F182Y, S188G, S190N, K191T, L192T, G193R, Q195Y, S201G, K225L, K229V, F232R, W234F, D236Q, and V238R, in reference to an I-OnuI LHE amino acid sequence as set forth in SEQ ID NOs: 1-5, or a biologically active fragment thereof.

26. The polypeptide of any one of claims 1 to 18, wherein the HE variant comprises at least 5, at least 15, preferably at least 25, more preferably at least 35, or even more preferably at least 40 or more of the following amino acid substitutions: S24W, L26S, R28V, N32S, K34T, S35V, S36K, S40R, E42L, G44S, Q46G, T48E, V68K, A70S, N75R, S78R, K80T, E85G, V116L, L138M, T143N, S159P, F168L, E178D, C180T, F182Y, S188G, S190N, K191T, L192T, G193R, Q195Y, S201G, K225L, K229V, F232R, W234F, D236Q, and V238R, in reference to an I-OnuI LHE amino acid sequence as set forth in SEQ ID NOs: 1-5, or a biologically active fragment thereof.

27. The polypeptide of any one of claims 1 to 18, wherein the HE variant comprises at least 5, at least 15, preferably at least 25, more preferably at least 35, or even more preferably at least 40 or more of the following amino acid substitutions: S24W, L26S, R28V, N32S, K34T, S35V, S36K, S40R, E42L, G44S, Q46G, T48E, V68K, A70S, N75R, S78R, K80T, E85G, V116L, L138M, T143N, S159P, F168L, E178D, C180T, F182Y, S188G, S190N, K191T, L192T, G193R, Q195Y, S201G, K225L, K229V, F232R, W234F, D236Q, and V238R, in reference to an I-OnuI LHE amino acid sequence as set forth in SEQ ID NOs: 1-5, or a biologically active fragment thereof.

28. The polypeptide of any one of claims 1 to 18, wherein the HE variant comprises at least 5, at least 15, preferably at least 25, more preferably at least 35, or even more preferably at least 40 or more of the following amino acid substitutions: S24W, R28V, N32S, K34T, S35V, S36K, S40R, E42L, G44S, Q46G, T48E, V68K, A70S, N75H, A76Y, S78R, K80T, T82S, V116L, L138M, T143N, S159P, F168L, E178D, C180T, F182Y, S188G, S190N, K191T, L192T, G193R, Q195Y, S201G, K225L, K229V, F232R, W234F, D236Q, and V238R, in reference to an I-OnuI LHE amino acid sequence as set forth in SEQ ID NOs: 1-5, or a biologically active fragment thereof.

29. The polypeptide of any one of claims 1 to 18, wherein the HE variant comprises at least 5, at least 15, preferably at least 25, more preferably at least 35, or even more preferably at least 40 or more of the following amino acid substitutions: S24W, L26S, R28V, N32S, K34T, S35V, S36K, S40R, E42L, G44S, Q46G, T48E, Q61R, V68K, A70S, N75R, S78R, K80T, V116L, L138M, T143N, S159P, F168L, E178D, C180S, F182Y, S188G, S190N, K191T, L192T, G193R, Q195Y, S201G, K225L, K229V, F232R, W234F, D236Q, and V238R, in reference to an I-OnuI LHE amino acid sequence as set forth in SEQ ID NOs: 1-5, or a biologically active fragment thereof.

30. The polypeptide of any one of claims 1 to 18, wherein the HE variant comprises at least 5, at least 15, preferably at least 25, more preferably at least 35, or even more preferably at least 40 or more of the following amino acid substitutions: S24W, R28D, N32S, K34T, S35V, S36K, S40R, E42L, G44S, Q46G, T48E, V68K, A70S, N75H, A76Y, S78R, K80T, T82S, V116L, L138M, T143N, K147E, S159P, I161V, F168L, E178D, C180T, F182Y, S188G, S190N, K191T, L192T, G193R, Q195Y, S201G, K225L, K229V, F232R, W234F, D236Q, and V238R, in reference to an I-OnuI LHE amino acid sequence as set forth in SEQ ID NOs: 1-5, or a biologically active fragment thereof.

31. The polypeptide of any one of claims 1 to 30, wherein the HE variant comprises an amino acid sequence that is at least 80%, preferably at least 85%, more preferably at least 90%, or even more preferably at least 95% identical to the amino acid sequence set forth in any one of SEQ ID NOs: 6-17, or a biologically active fragment thereof.

32. The polypeptide of any one of claims 1 to 31, wherein the HE variant comprises the amino acid sequence set forth in SEQ ID NO: 6, or a biologically active fragment thereof.

33. The polypeptide of any one of claims 1 to 31, wherein the HE variant comprises the amino acid sequence set forth in SEQ ID NO: 7, or a biologically active fragment thereof.

34. The polypeptide of any one of claims 1 to 31, wherein the HE variant comprises the amino acid sequence set forth in SEQ ID NO: 8, or a biologically active fragment thereof.

35. The polypeptide of any one of claims 1 to 31, wherein the HE variant comprises the amino acid sequence set forth in SEQ ID NO: 9, or a biologically active fragment thereof.

36. The polypeptide of any one of claims 1 to 31, wherein the HE variant comprises the amino acid sequence set forth in SEQ ID NO: 10, or a biologically active fragment thereof.

37. The polypeptide of any one of claims 1 to 31, wherein the HE variant comprises the amino acid sequence set forth in SEQ ID NO: 11, or a biologically active fragment thereof.

38. The polypeptide of any one of claims 1 to 31, wherein the HE variant comprises the amino acid sequence set forth in SEQ ID NO: 12, or a biologically active fragment thereof.

39. The polypeptide of any one of claims 1 to 31, wherein the HE variant comprises the amino acid sequence set forth in SEQ ID NO: 13, or a biologically active fragment thereof.

40. The polypeptide of any one of claims 1 to 31, wherein the HE variant comprises the amino acid sequence set forth in SEQ ID NO: 14, or a biologically active fragment thereof.

41. The polypeptide of any one of claims 1 to 31, wherein the HE variant comprises the amino acid sequence set forth in SEQ ID NO: 15, or a biologically active fragment thereof.

42. The polypeptide of any one of claims 1 to 31, wherein the HE variant comprises the amino acid sequence set forth in SEQ ID NO: 16, or a biologically active fragment thereof.

43. The polypeptide of any one of claims 1 to 31, wherein the HE variant comprises the amino acid sequence set forth in SEQ ID NO: 17, or a biologically active fragment thereof.

44. The polypeptide of any one of claims 1 to 43, wherein the HE variant binds a polynucleotide sequence in the BTK gene.

45. The polypeptide of any one of claims 1 to 44, wherein the HE variant binds the polynucleotide sequence set forth in SEQ ID NO: 24.

46. The polypeptide of any one of claims 1 to 45, further comprising a DNA binding domain.

47. The polypeptide of claim 46, wherein the DNA binding domain is selected from the group consisting of: a TALE DNA binding domain and a zinc finger DNA binding domain.

48. The polypeptide of claim 47, wherein the TALE DNA binding domain comprises about 9.5 TALE repeat units to about 15.5 TALE repeat units.

49. The polypeptide of claim 47 or claim 48, wherein the TALE DNA binding domain binds a polynucleotide sequence in the BTK gene.

50. The polypeptide of any one of claims 47 to 49, wherein the TALE DNA binding domain binds the polynucleotide sequence set forth in SEQ ID NO: 25.

51. The polypeptide of claim 47, wherein the zinc finger DNA binding domain comprises 2, 3, 4, 5, 6, 7, or 8 zinc finger motifs.

52. The polypeptide of any one of claims 1 to 51, further comprising a peptide linker and an end-processing enzyme or biologically active fragment thereof.

53. The polypeptide of any one of claims 1 to 52, further comprising a viral self-cleaving 2A peptide and an end-processing enzyme or biologically active fragment thereof.

54. The polypeptide of claim 52 or claim 53, wherein the end-processing enzyme or biologically active fragment thereof has 5′-3′ exonuclease, 5′-3′ alkaline exonuclease, 3′ 5′ exonuclease, 5′ flap endonuclease, helicase, template-dependent DNA polymerase or template-independent DNA polymerase activity.

55. The polypeptide of any one of claims 52 to 54, wherein the end-processing enzyme comprises Trex2 or a biologically active fragment thereof.

56. The polypeptide of any one of claims 1 to 55, wherein the polypeptide cleaves the human BTK gene at the polynucleotide sequence set forth in SEQ ID NO: 24 or SEQ ID NO: 26.

57. A polynucleotide encoding the polypeptide of any one of claims 1 to 56.

58. An mRNA encoding the polypeptide of any one of claims 1 to 56.

59. A cDNA encoding the polypeptide of any one of claims 1 to 56.

60. A vector comprising a polynucleotide encoding the polypeptide of any one of claims 1 to 56.

61. A cell comprising the polypeptide of any one of claims 1 to 56.

62. A cell comprising a polynucleotide encoding the polypeptide of any one of claims 1 to 56.

63. A cell comprising the vector of claim 60.

64. A cell comprising one or more genome modifications introduced by the polypeptide of any one of claims 1 to 56.

65. The cell of any one of claims 61 to 64, wherein the cell is a hematopoietic cell.

66. The cell of any one of claims 61 to 65, wherein the cell is a hematopoietic stem or progenitor cell.

67. The cell of any one of claims 61 to 66, wherein the cell is a CD34⁺ cell.

68. The cell of any one of claims 61 to 67, wherein the cell is a CD133⁺ cell.

69. A composition comprising a cell according to any one of claims 61 to 68.

70. A composition comprising the cell according to any one of claims 61 to 68 and a physiologically acceptable carrier.

71. A method of editing a BTK gene in a cell comprising: introducing the polypeptide of any one of claims 1 to 56, the polynucleotide encoding the polypeptide of any one of claims 57 to 59, or the vector of claim 60; and a donor repair template into the cell, wherein expression of the polypeptide creates a double strand break at a target site in a BTK gene and the donor repair template is incorporated into the BTK gene by homology directed repair (HDR) at the site of the double-strand break (DSB).

72. The method of claim 71, wherein the BTK gene comprises one or more amino acid mutations or deletions that result in X-linked agammaglobulinemia (XLA).

73. The method of claim 71 or claim 72, wherein the cell is a hematopoietic cell.

74. The method of any one of claims 71 to 73, wherein the cell is a hematopoietic stem or progenitor cell.

75. The method of any one of claims 71 to 74, wherein the cell is a CD34⁺ cell.

76. The method of any one of claims 71 to 75, wherein the cell is a CD133⁺ cell.

77. The method of any one of claims 71 to 76, wherein the polynucleotide encoding the polypeptide is an mRNA.

78. The method of any one of claims 71 to 77, wherein a polynucleotide encoding a 5′-3′ exonuclease is introduced into the cell.

79. The method of any one of claims 71 to 78, wherein a polynucleotide encoding Trex2 or a biologically active fragment thereof is introduced into the cell.

80. The method of any one of claims 71 to 79, wherein the donor repair template comprises a 5′ homology arm homologous to a BTK gene sequence 5′ of the DSB, a donor polynucleotide, and a 3′ homology arm homologous to a BTK gene sequence 3′ of the DSB.

81. The method of claim 80, wherein the donor polynucleotide is designed to repair one or more amino acid mutations or deletions in the BTK gene.

82. The method of claim 80, wherein the donor polynucleotide comprises a cDNA encoding a BTK polypeptide.

83. The method of claim 80, wherein the donor polynucleotide comprises an expression cassette comprising a promoter operable linked to a cDNA encoding a BTK polypeptide.

84. The method of claim 80, wherein the donor polynucleotide comprises an to a cDNA encoding a BTK polypeptide operably linked to a post-transcriptional response element and a polyadenylation sequence.

85. The method of any one of claims 80 to 84, wherein the lengths of the 5′ and 3′ homology arms are independently selected from about 100 bp to about 2500 bp.

86. The method of any one of claims 80 to 85, wherein the lengths of the 5′ and 3′ homology arms are independently selected from about 600 bp to about 1500 bp.

87. The method of any one of claims 80 to 86, wherein the 5′homology arm is about 1500 bp and the 3′ homology arm is about 1000 bp.

88. The method of any one of claims 80 to 87, wherein the 5′homology arm is about 600 bp and the 3′ homology arm is about 600 bp.

89. The method of any one of claims 80 to 88, wherein a viral vector is used to introduce the donor repair template into the cell.

90. The method of claim 89, wherein the viral vector is a recombinant adeno-associated viral vector (rAAV) or a retrovirus.

91. The method of claim 90, wherein the rAAV has one or more ITRs from AAV2.

92. The method of claim 90 or claim 91, wherein the rAAV has a serotype selected from the group consisting of: AAV1, AAV2, AAV3, AAV4, AAV5, AAV6, AAV7, AAV8, AAV9, and AAV 10.

93. The method of any one of claims 90 to 92, wherein the rAAV has an AAV2 or AAV6 serotype.

94. The method of claim 90, wherein the retrovirus is a lentivirus.

95. The method of claim 94, wherein the lentivirus is an integrase deficient lentivirus (IDLY).

96. A method of treating, preventing, or ameliorating at least one symptom of X-linked agammaglobulinemia (XLA), or condition associated therewith, comprising harvesting a population of cells from the subject; editing the population of cells according to the method of any one of claims 71 to 95, and administering the edited population of cells to the subject.
